(19) United States
(12) Patent Application Publication
(10) Pub. No.: US 2003/0045710 A1
(43) Pub. Date: Mar. 6, 2003

(54) Methods for identifying, obtaining and using new polyketide compounds

(76) Inventors: Thomas C. Bruice, Santa Barbara, CA (US); Helgi Adalsteinsson, Livermore, CA (US); Kalju Kahn, Goleta, CA (US)

Correspondence Address: MANDEL & ADRIANO, 35 NORTH ARROYO PARKWAY, SUITE 60, PASADENA, CA, 91103, US

(21) Appl. No.: 10/098,863

(22) Filed: Mar. 15, 2002

Related U.S. Application Data
(63) Continuation-in-part of application No. 09/544,503, filed on Apr. 6, 2000.

(51) Int. Cl. C07D 491/16; G01N 33/53; G06G 7/48; G06G 7/58; A61K 31/4745

(52) U.S. Cl. 540/456; 540/457; 703/12; 435/7.1; 514/291

(57) ABSTRACT

The invention provides novel polyketide compounds having desired bioactivity, including neuroregenerative activity without immunosuppressive activity, and methods of making and using these compounds.

This application is a continuation-in-part of U.S. Ser. No. 09/544,503, filed Apr. 6, 2000, the contents of which are incorporated by reference in their entirety into the present application.

Throughout this application various publications are referenced. The disclosures of these publications in their entireties are hereby incorporated by reference into this application in order to more fully describe the state of the art to which this invention pertains.

### FIELD OF THE INVENTION

The present invention relates to methods of identifying and obtaining novel polyketide compounds having desired properties, by generating and screening virtual libraries of polyketide compounds, and producing candidate compounds using biosynthetic procedures, and more particularly to methods of generating modified polyketide compounds to obtain neuroregenerative agents, and the agents so obtained and methods of using these agents.

### BACKGROUND OF THE INVENTION

Polyketides are natural products whose biosynthesis takes place through successive Claisen condensations of simple carboxylates, e.g. acetate, propionate, or malonyl or methylmalonyl. Naturally occurring polyketides (native polyketides) include a large number of bioactive compounds including antibiotic, antifungal, and antitumor drugs. Polyketides can vary widely in size, from the eight carbon 6-methylsalicylic acid up to maitotoxin which, with a molecular weight of 3422, is one of the largest natural products known. (Murata et al., J. Am. Chem. Soc., 1993, 115:2060-2062). Polyketides can contain aromatic rings, branched chains, polycycles, and a variety of other structural features.

The biosynthesis of polyketides shares many characteristics with the biosynthesis of fatty acids. The ketide chain grows by two carbon atom increments during each condensation cycle, and modifications to each ketide unit follow immediately after the condensation step.

Unlike fatty acid synthesis, the reduction of the β-carbonyl group in the newly added ketide unit is not required following the condensation step. As a result, the polyketide chain formed can contain a combination of carbonyl groups, alcohols, double bonds, or methylene units. Additionally, successive condensations can add a variety of different carboxylates, resulting in a large number of possible substituents with varied stereochemistry. This variability in the nature and modification to the substituents results in a virtually endless number of possible polyketides.

Compounds that bind to the immunophilin protein FKBP12 have attracted attention in recent years due to the neuroregenerative abilities described for several such ligands. (Hamilton and Steiner, J. Med. Chem., 1998, 41:5119-5143). The best known FKBP12 ligands are the polyketides FK506 and Rapamycin, which bind in an identical way to FKBP12 through structurally similar regions of the drugs known as the binding domain (FIG. 1). (Babine and Bender, Chem. Rev. 1997, 97:1359-1472). Most synthetic FKBP12 ligands contain the pipecolinic and dicarbonyl portions of the binding domains of Rapamycin (FIG. 2) and FK506 (FIG. 18).

The binding domains of Rapamycin and FK506 have been proposed to act as proline-leucine mimics. (Albers et al., J. Org. Chem., 1990, 55:4984-4986; Ikeda et al., J. Am. Chem., 1994, 116:4143-4144). Structural features resembling the binding domains of Rapamycin and FK506 are found in a large number of known FKBP12 ligands.

The FKBP12-FK506 complex subsequently binds to the protein calcineurin, and the FKBP12-Rapamycin complex binds to a protein known as FRAP. The binding of the second protein to the FKBP12-drug complexes is responsible for the immunosuppressive activities of the drugs. (Schreiber, Science, 1991, 251:283-287; Dumont et al., J. Exp. Med., 1992, 176:751-760; Bierer et al., Science 1990, 250:556-559). Modifications to FK506 or Rapamycin that preclude binding to calcineurin or FRAP, respectively, lead to loss of immunosuppressive activity.

The binding of the FKBP12-Rapamycin and FKBP12-FK506 complexes to FRAP and calcineurin, respectively, occur at structural regions of the drugs known as the effector domains (FIG. 1). The effector domains of the drugs differ greatly, as is required by their different target proteins. The effector domain of Rapamycin has been proposed to act as a peptide mimic in binding to FRAP. (Odagaki and Clardy, J. Am. Chem. Soc., 1997, 119:10253-10254). It has been reported that the interactions between Rapamycin and FRAP are mainly hydrophobic interactions with the three adjacent double bonds in the effector domain of Rapamycin (FIG. 1). (Babine, Chem. Rev. 1997, 97:1359-1472).

Rapamycin and FK506 promote neurite outgrowth as secondary activities (Lyons et al., Proc. Natl. Acad. Sci. USA 1994, 91:3191-3195). Furthermore, the immunosuppressive and neurite outgrowth activities of these drugs can be separated: the toxicity of these compounds is related to their immunosuppressive abilities while neuroregenerative activity is retained by non-immunosuppressive agents which bind to FKBP12 but not to the effector proteins calcineurin or FRAP. (Holt, J. Am. Chem. Soc. 1993, 115:9925-9938). Synthetic, non-immunosuppressive FKBP12 ligands have been shown to promote nerve regrowth in vitro and in vivo without the addition of exogenous growth factors. (Hamilton et al., Bioorg. Med. Chem. Let. 1997, 7:1785-1790; Steiner et al., Proc. Natl. Acad. Sci. USA 1997, 94:2019-2024; Steiner et al., Nature Medicine 1997, 3:421; Hamilton and Steiner, J. Med. Chem., 1998, 41:5119-5143). Several synthetic FKBP ligands that resemble the binding domain of FK506 and Rapamycin have also been shown to have neuroregenerative abilities, as has been reviewed by Hamilton and Steiner (Hamilton and Steiner, supra). Recent studies indicate that the neuroregenerative properties of these compounds are due to their ability to bind to the protein FKBP52 (Gold et al., J. Pharm. Exp. Ther., 1999, 289:1202-1210). (Craescu et al., J. Biochem., 1996, 35:11045-52). Far less is known about the structure of FKBP52 than the structure of FKBP12, but it has been shown that the structure of the FK506 binding site of FKBP59 differs by less than 1 Å from the binding site of FKBP12. (Saunders, J. Am. Chem. Soc. 1987, 109:3150). A strong, albeit not fully linear, correlation is also seen between FKBP12 binding affinity and neuroregenerative ability (Hamilton and Steiner, supra).

The enzymes responsible for the biosynthesis of polyketides, including the drugs Rapamycin and FK506, are called polyketide synthases (PKSs). The polyketide synthases are modular enzymes, in which multiple catalytic units (modules) are assembled into a cluster responsible for the total synthesis of the polyketide chain. Each module catalyzes a specific reaction, forming a molecular assembly line in which successive modifications contribute to the formation of the final product. The cloning of the polyketide synthase responsible for the synthesis of Erythromycin provided the first insight into the nature of a modular polyketide synthase, as well as the first opportunity to modify their function. (Cortes et al., Nature 1990, 348:176-178; Donadio et al, Science, 1991, 252:675-679). Modifications performed on the Erythromycin PKS include modifications to the oxidation states of the ketides in the macrocycle, deletions of ketide units, and the inclusion of synthetic non-ketide units through the interruption of the polyketide synthesis, as has been summarized in reviews by Katz (Chem. Rev., 1997, 97:2557-2575) and Khosla (Chem. Rev., 1997, 2577-2590). This technique of chemobiosynthesis has been used successfully to alter PKS genes in order to make new unnatural polyketide compounds (McDaniel, R., et al. Science, 1993, 262, 1546-1550).

The biosynthetic gene cluster responsible for synthesizing Rapamycin has been sequenced, (Scwecke et al., Proc. Natl. Acad. Sci. USA 1995, 92:7839-7843) making engineered modifications to Rapamycin feasible. Three clustered genes were found to be responsible for generating the Rapamycin PKS, which together encode 14 modules of the PKS cluster. Each unit in the cluster was found to be responsible for a single round of chain elongation, and an adjacent gene was found to encode a pipecolate incorporating enzyme, which completes the macrocycle. In all, 70 constituent active sites are involved in the biosynthesis of Rapamycin. FIG. 3 contains a schematic drawing of the ketide units added by each module of the PKS cluster. The thick bonds indicate atoms that are added to the product in each module, starting with the cyclohexyl ring in the cyclohexyl arm (FIG. 1) and progressing toward the pipecolate ring (FIG. 2), from work by Scwecke et al. (Proc. Nat. Acad. Sci. USA 92:7839 (1995)).

There remains a need for polyketide compounds having desired bioactivity, including compounds having neuroregenerative properties, and for methods of obtaining such compounds.

### SUMMARY OF THE INVENTION

Accordingly, the present invention provides novel polyketide compounds for use as biologically active agents. The compounds are obtained by the methods of the invention in which computer simulations are used to establish a virtual library of potentially bioactive polyketide compounds, from which candidate compounds are selected and then prepared using, for example, biosynthetic procedures. The compounds, for example Rapamycin and/or FK506 analogs, demonstrating bioactivity in suitable tests, are then used in methods, such as methods to regenerate neurons by contacting the neurons with a selected neuroregenerative Rapamycin and/or FK506 analog.

In a method of the invention, computer simulations are used to generate a virtual combinatorial polyketide library of analogs of known, biologically active native polyketide compounds such as Rapamycin and FK506, and to then identify compounds that have the desired bioactivity. For identification, the library is screened using computer-aided searching to identify candidate compounds that are expected to exhibit the desired bioactivity, and that are suitable for production by biosynthesis. The bioactivity can be an activity such as binding to a selected compound, or absence of an activity, such as absence of binding to a selected compound.

The methods of the present invention provide modifications to polyketide compounds, such as Rapamycin and/or FK506, that yield non-natural analogs which: (i) bind to FKBP12 in an identical manner as Rapamycin and or FK506; (ii) do not form the ternary FKBP12-ligand-FRAP complex of Rapamycin; (iii) are accessible by engineered biosynthesis through modifications of the PKS responsible for generating Rapamycin; and (iv) possess neuroregenerative properties.

In an embodiment of the invention, constrained cyclic analogs of FK506 and/or Rapamycin are generated by virtual deletions or exchanges of ketide units in the effector domains of FK506 and Rapamycin, or analogs of these compounds. The modifications to the effector domains are obtained by point mutations and/or replacement of one or more ketide units. The virtual library of analogs is screened to identify candidate compounds with ground state conformation complementary to the FKBP-12 binding domain, but not to effector proteins, and which are suitable for production by modified polyketide synthases that are responsible for generating the native compound, Rapamycin.

In another embodiment of the invention, the effector domain of a native, bioactive polyketide is replaced by a short polyketide tether with substituents placed in such a way to favor binding to selected compounds, for example to favor holding the binding domain of the ligand in a conformation ideally suited for binding to FKBP12.

Candidate compounds are prepared by biosynthetic procedures, and tested for the desired bioactivity, for example using binding and cell culture assays. Using these methods, novel compounds having desired bioactivity are obtained.

### BRIEF DESCRIPTION OF THE DRAWINGS

FIG. 1 depicts the structure of Rapamycin showing the binding domain, effector domain, and cyclohexyl arm, as described, infra. The atomic numbers shown correspond to the standard numbering of atoms in Rapamycin.

FIG. 2 depicts the structure of Rapamycin having individual ketide units that are joined by the biosynthetic gene cluster responsible for generating the drug. The thick bonds in the structure correspond to the individual ketide units added at each step by the polyketide synthases. The biosynthesis starts at the cyclohexyl ring in the rap A gene cluster and progresses toward the pipecolate ring.

FIG. 3 shows the cassette of possible ketide units used in the combinatorial substitutions, as described in Example II, infra.

FIG. 4 shows a comparison of the torsional energy barriers for bond rotation around the C—C(═O)—C(═O)—N bond in N,N-dimethyl-3-hydroxyl-2-oxopropionamide calculated using HF/6-31G(d) (o) and Amber parameters devised using PM3 charges for the 2-oxopropionamide (+).

FIG. 5 is a comparison of rotational energy barrier around the C2=C3-C4=C5 torsion angle in 1,2-hexatriene using HF/6-31G(d) (o) and the Amber parameters of Hermone et al, (Biochemistry, 1998, 37: 2843) with PM3 charges (+). The torsion being varied is indicated by a thick bond in the inset structure.

FIG. 6 depicts the structures of modified Rapamycin analogs examined in Example I, infra.

FIG. 7 is a space filling representation of Rapamycin and the modified Rapamycin analogs RP1-RP8 docked in the binding pocket of FKBP 12.

FIG. 8 is a tube representation of the structure of RP7 with the binding domain in the conformation required for binding to FKBP12.

FIG. 9 A and B depicts the mobilities of Rapamycin (9A) and RP8 (9B) bound to FKBP12. The mobilities are measured as RMS deviation from the average structures of the Rapamycin analogs during the simulations, as described in Example I, infra.

FIG. 10 shows binary free energy profiles for Rapamycin and several Rapamycin analogs to FKBP 12 (RP 1-RP8) using Linear Interaction Energies, as described in Example I, infra.

FIG. 11 shows ternary energy binding profiles for Rapamycin and some of the truncated Rapamycin analogs (RP1-RP5) with FKBP12 and FRAP using Linear Interaction Energies, as described in Example I, infra.

FIG. 12 are examples of the substituted Rapamycin analogs examined in Examples I and II, infra. The dotted lines indicate the division of the tethers into individual ketide units, showing where substitutions were performed to generate novel tethers.

FIG. 13 shows the template Rapamycin analogs generated by replacing the effector domains of the Rapamycin analogs in FIG. 12 with methylene chains, as described in Example II, infra.

FIG. 14 is a bar graph showing the location of the lowest energy binding conformation of a tetraketide that does not favor conformations capable of binding to FKBP12. An arrow is used to indicate the location of the lowest energy binding conformation.

FIG. 15 is a bar graph showing the location of the lowest energy binding conformation of a tetraketide that favors conformations capable of binding to FKBP12. An arrow is used to indicate the location of the lowest energy binding conformation.

FIG. 16 shows a stereo view of the favored binding conformation of compound V—B to FKBP12. The binding domain of the Rapamycin analog is at the bottom at the picture.

FIG. 17 shows the four Rapamycin analogs designed as optimal FKBP12 binders (V-B, IV-A, X, XI) as described in Example II, infra.

FIG. 18 depicts the structure of FK506 showing the binding domain, effector domain, and cyclohexyl arm, as described, infra.

FIG. 19 depicts structures of methoxymethanol (MMeOH), methoxyethanol (MetOH), tetrahydropyra-2-ol (THPOH), 3-hudroxy-2-oxopropanol (HOP), 3-methoxy-2-oxopropanol (MOP), 3-hydroxy-3-methoxy-2-oxopropanal (HMOP), 2-glyoxoyl-tetrahydropyran (GTHP), 2-glyoxoyl-tetrahydropyran-2-ol (GTHPO), methyl formate, methyl acetate, methyl propionate, ethyl formate, propyl formate, isopropyl formate, and a pipecolinyl ester (MPIP).

FIG. 20 depicts the MP2/aug-cc-pVTZ optimized structures of alkanes, alcohols, ethers, esters, and hemiacetals for the determination of bond stretching and angle bending parameters. Selected C—C and C—O distances are shown in Å.

FIG. 21 depicts the structures of modified tetraketide analogs of FK506 as described in Example III, infra.

FIG. 22 depicts the lowest energy conformation of a tetraketide C (shown in FIG. 21C) docked to the binding site of the rabbit FKBP52 protein. The pipecolinyl ring and ketoamide moiety lie at the bottom of the cavity with the linker region protruding from the protein. The image was produced with the program MOLEKEL (Portmann, S. and Lüithi, H. P. CHIMIA 2000, 54, 766-770).

FIG. 23 depicts conformers and their relative energies (in kcal/mol) of 1-propanol at the MP2/aug-cc-pVTZ//MP2/aug-cc-pVTZ level.

FIG. 24 depicts conformers and their relative energies (in kcal/mol) of 2-butanol at the MP2/TZ//DZ level.

FIG. 25 depicts Conformers and their relative energies (in kcal/mol) of methyl propyl ether at the MP2/TZ/DZ level.

FIG. 26 depicts Conformers and their relative energies (in kcal/mol) of methoxymethanol at the MP2/aug-cc-pVTZ//MP2/aug-cc-pVTZ level.

FIG. 27 depicts torsional profiles for methoxymethanol. Panel A: OC—OH rotation with OC—OC anti. Panel B: OC—OH rotation with OC—OC gauche⁺. Panel C: OC—OC rotation with OC—OH gauche⁺. Open circles-MP2/aug-cc-pVDZ, closed circles-MP2/TZ//DZ, solid line—OPLS-AA.

FIG. 28 depicts conformers and their relative energies (in kcal/mol) of (R)-1-methoxyethanol at the MP2/TZ//DZ level.

FIG. 29 depicts conformers and their relative energies (in kcal/mol) of tetrahydropyran-2-ol at the MP2/aug-cc-pVDZ level. For visual comparison, axial conformers are shown in the S configuration and equatorial conformers are shown in the R configuration.

FIG. 30 depicts torsional profile for the NC—CO rotation in the pipecolinyl ester. Open circles—MP2/6-31+G(d,p), solid line—OPLS-AA.

FIG. 31 depicts torsional profile for the CC—CO rotation in methyl propionate. Open circles—MP2/aug-cc-pVDZ, closed circles-MP2/TZ//DZ, solid line—OPLS-AA.

FIG. 32 depicts conformers and their relative energies (in kcal/mol) of 3-hydroxy-2-oxopropanal at the MP2/DZ//TZ level.

FIG. 33 depicts conformers and their relative energies (in kcal/mol) of 3-methoxy-2-oxopropanal at the MP2/DZ//TZ level.

FIG. 34 depicts torsional profiles for 3-hydroxy-2-oxopropanal. Panel A: O═C—C═O rotation with CC—OH anti. Panel B: CC—OH rotation with O═C—C═O s-trans. Panel C: O═C—C—O rotation with O═C—C═O s-trans. Open circles—MP2/aug-cc-pVDZ, closed circles—MP2/TZ//DZ, solid line—OPLS-AA.

FIG. 35 depicts torsional profiles for 3-methoxy-2-oxopropanal. Panel A: O═C—C═O rotation with CC—OC anti. Panel B: CC—CO rotation with O═C—C═O s-cis. Panel C: CC—CO rotation with O═C—C═O s-trans. Panel D: CC—OC rotation with O═C—C═O s-trans. Panel E: O═C—C—O rotation with O═C—C═O s-trans and O—CH₃ gauche. Open circles—MP2/aug-cc-pVDZ, closed circles—MP2/TZ//DZ, solid line—OPLS-AA.

FIG. 36 depicts conformers and their relative energies (in kcal/mol) of 3-hydroxy-3-methoxy-2-oxopropanal at the MP2/TZ//DZ level.

FIG. 37 depicts Conformers and their relative energies (in kcal/mol) of 2-glyoxoyl-tetrahydropyran at the MP2/aug-cc-pVDZ//6-31+G(d,p) level.

FIG. 38 depicts Conformers and their relative energies (in kcal/mol) of 2-glyoxoyl-tetrahydropyran-ol at the MP2/aug-cc-pVDZ//6-31+G(d,p) level.

### DETAILED DESCRIPTION OF THE INVENTION

The present invention provides novel polyketide compounds obtained by modifications in the ketides units of the effector domains of polyketide compounds to provide compounds having desired bioactivity. Polyketides can vary widely in size and structure. Polyketides can contain aromatic rings, branched chains, polycycles, and a variety of other structural features. Examples of polyketide compounds include, but are not limited to Rapamycin, FK506, Ascomycin (Liu, J. et al., Biochemistry, 1992, 31: 3896-3901), L685,818 (Steiner, J. P. Nature Medicine, 1997, 3(4): 421-428), 506BD (Somers, P. K. et al., J. Am. Chem. Soc. 1991, 113: 8045-8056; Bierer, B. E. et al., Science, 1990, 250: 556-559), acyclic 506BD (Somers, P. K. et al., J. Am. Chem. Soc., 1991, 113: 8045-8056) and Erythromycin.

Naturally occurring polyketides can be modified to obtain compounds that possess the desired bioactivity. As an example, the polyketide Rapamycin can be modified to obtain compounds that possess neuroregenerative activity, and not immunosuppressive properties. In another embodiment, the polyketide FK506 is modified to obtain compounds that possess neurogenerative activity, and not immunosuppressive activity. Native Rapamycin and FK506 possess both immunosuppressive and neuroregenerative properties.

In the methods of the invention, novel polyketide compounds are obtained by generating and screening of “virtual” combinatorial libraries of analogs of a known “native” polyketide compound having desired properties, for example Rapamycin or FK506, using computer programs and methods, such as Amber 5.0 (Case et al., 1997, University of California, San Francisco), ChelpG (Breneman, C. M.; Wiberg, K. B. J. Comp. Chem., 1990, 11, 361-373), Gaussian 94 (Frisch et al., 1995, Gaussian Inc., Pittsburgh, Pa.) and AM1 (Dewar et al., J. Am. Chem. Soc., 1985, 107, 3902-3909).

The polyketide linker in the effector domain of FK506 analogs may be a triketide, a tetraketide, a pentaketide, a hexaketide, or a heptaketide. In a preferred embodiment, the polyketide linker in the effector domain of FK506 analogs is a tetraketide.

The polyketide linker in the effector domain of rapamycin analogs may be a triketide, a tetraketide, a pentaketide, a hexaketide, a heptaketide, an octaketide, a nonaketide, a decaketide, or a undecaketide.

Two approaches are taken to generate a virtual combinatorial library of Rapamycin and/or FK506 analogs of modified effector domain including: (1) deletions of one or more ketide units from the effector domain; and (2) replacement of one or more ketide units in the effector domain by different ketide units.

Computer modeling is used to provide modifications to polyketide compounds, for example, the natural polyketide drugs, Rapamycin and/or FK506, to obtain polyketide compounds having desired properties, such as suppression of its immunosuppressive abilities, while retaining neuroregenerative abilities.

In an embodiment for obtaining analogs of Rapamycin and/or FK506, the modifications used are designed to correspond to deletions in the modular polyketide synthase responsible for generating Rapamycin and/or FK506, so that the desired new compounds can be produced by engineered biosynthesis, i.e. genetic manipulation of PKS modules. In one embodiment, the modifications are successive single ketide deletions from the effector domain of Rapamycin to produce neuroregenerative analogs. The resulting compounds bind to agents such as FKBP12, which is a requirement for neuroregenerative activity, but not to FRAP (thus eliminating immunosuppressive ability).

In another embodiment, substitutions of ketide units are used to replace the effector domain of Rapamycin and/or FK506. For example, the effector domain is replaced by methylene tethers. In this embodiment, the resulting compounds retain the neuroregenerative abilities of Rapamycin, have a binding affinity (calculated linear interaction energy) to FKBP 12 identical to that of Rapamycin, and are suitable for synthesis by modified polyketide synthases. The replacement of the effector domain with short polyketide tethers, with substituents placed so as to favor holding the binding domain of the Rapamycin analog in a conformation ideally suited for binding to FKBP12, provides several advantages over direct modifications to native Rapamycin. First, the novel polyketide Rapamycin analogs are too small to protrude from the surface of FKBP12, thereby avoiding the possibility of binding to an effector protein. Second, the novel FKBP12 binding compounds may have very high affinities because the binding domain of the Rapamycin analog is optimized so as to be properly prepositioned to bind to FKBP12.

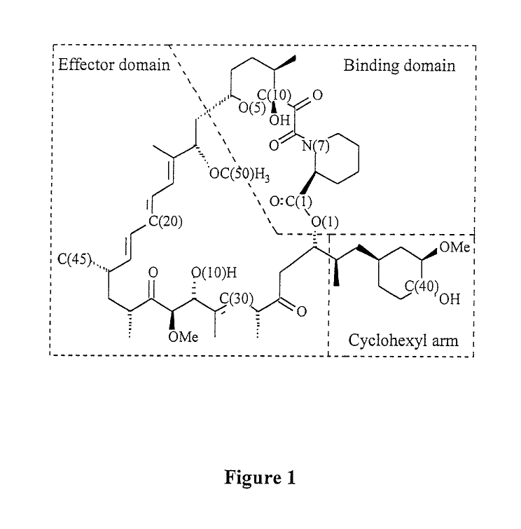

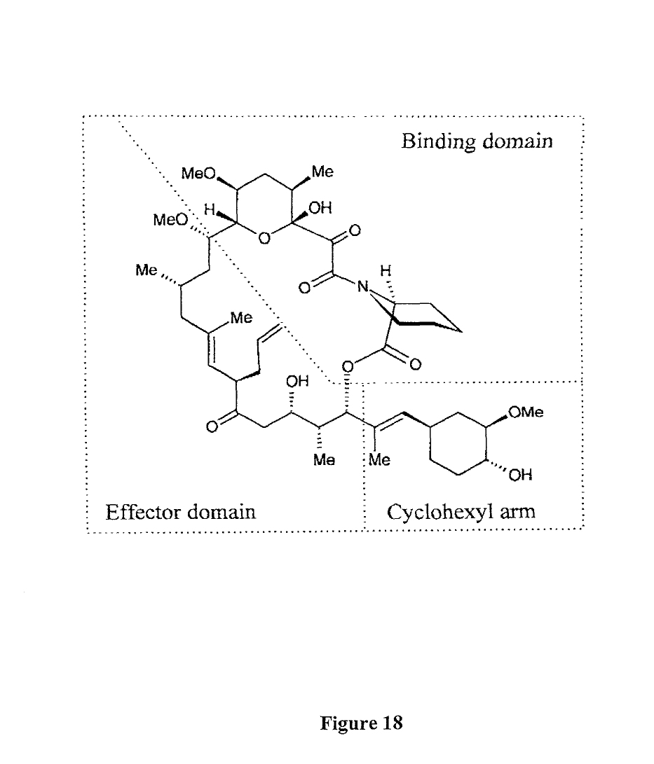

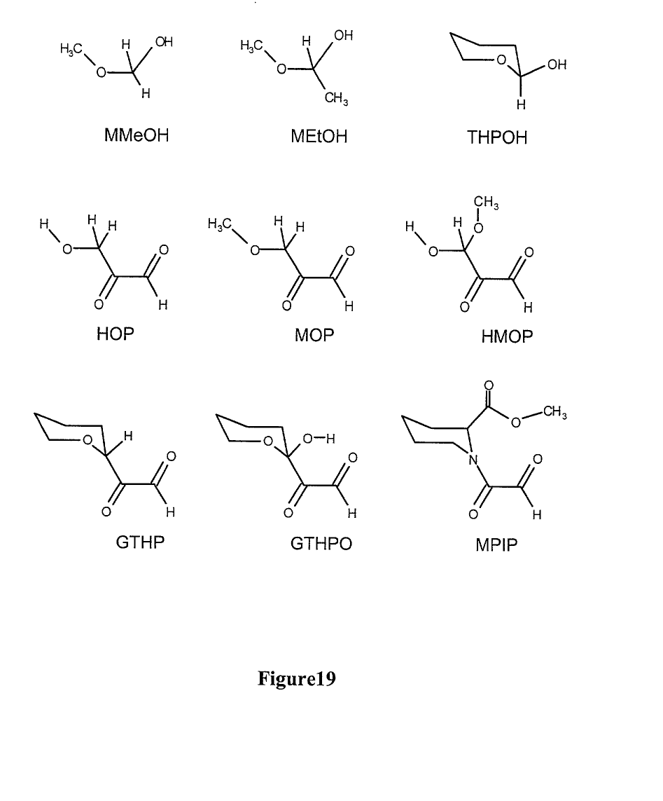

With respect to the method of the invention to obtain analogs of Rapamycin and/or FK506, that retain only the neuroregenerative properties, the selection criteria applied to obtain putative neuroregenerative agents are: (a) the binding domain of the compounds that will function as Rapamycin and/or FK506 analogs for FKBP12 must favor an orientation that is complimentary to the FKBP12 binding pocket; (b) the tether linking the two ends of the binding domain must not favor a position in which it protrudes into areas occupied by the enzyme; and (c) the modified Rapamycin and/or FK506 analog must favor a conformation of the cyclohexyl arm (FIGS. 1 and 18) corresponding to that of Rapamycin and/or FK506 bound to FKBP12.

Application of the methods of the invention involves computer simulations to obtain libraries of candidate polyketide compounds. Computer simulation of biomolecules and large organic molecules utilizes molecular mechanics calculations. For example, several high quality force fields, such as AMBER (Weiner, S. J. et al., J Am Chem Soc 1984, 106, 765-784; Cornell, W. D. et al., J Am Chem Soc 1995, 117, 5179-5197), CFF (Rasmussen, K.; Engelsen, S. B. “The Consistent Force Field: Development of potential energy functions for conformational analysis” in Recent Experimental and Computational Advances in Molecular Spectroscopy (Fausto, R., Ed.;), Kluwer Academic Publishers: Dordrecht, 1993; Vol. 406, pp. 381-419;Ewig, C. S. et al., J Phys Chem B 1999, 103: 6998-7014), CHARMM (Brooks, B. R. et al., J Comput Chem 1983, 4, 187-217; MacKerell, A. D., Jr. et al., J Phys Chem B 1998, 102, 3586-3616), COMPASS (Sun, H. J Phys Chem B 1998, 102, 7338-7364), GROMOS (Scott, W. R. P. et al., J Phys Chem A 1999, 103, 3596-3607), SPASIBA (Derreumaux, P. and Vergoten, G. J Chem Phys 1995, 102, 8586-8605), MM3 (Allinger, N. L. et al., J Am Chem Soc 1989, 111, 8551-8566), MM4 (Allinger, N. L. et al., J Comput Chem 1996, 17, 642-668), OPLS-AA (Jorgensen, W. L. et al., J Am Chem Soc 1996, 118, 11225-11236), and MMFF94 (Halgren, T. A. J Comput Chem 1999, 20, 730-748; Halgren, T. A. J Comput Chem 1996, 17, 490-519) have been developed and are commonly used for elucidating structures and properties of molecules in gas, liquid, and crystal phases. Each force field has significant strength in the area where it was specifically parameterized, e.g., MMFF94 performs well for predicting the structures and relative conformational energies of a wide range of organic molecules while the OPLS-AA potential is well suited for describing intermolecular interactions (Halgren, T. A. J Comput Chem 1999, 20, 730-748; Kaminski, G. and Jorgensen, W. L. J Phys Chem 1996, 100, 18010-18013).

The computer simulation method of the invention employs a parameterized force field, e.g., OPLS-AA (Jorgensen, W. L. et al., J Am Chem Soc 1996, 118:11225-11236) for the conformational analysis of macrocyclic polyketides. The parameterization involves improving the existing torsional parameters for alkanes, alcohols, ethers, hemiacetals, esters, and ketoamides, e.g., based on MP2/aug-cc-pVTZ and MP2/aug-cc-pVDZ calculations. In one embodiment, nonbonded parameters for the sp³ carbon and oxygen atoms can be refined using Monte Carlo simulations of bulk liquids. The resulting force field predicts conformer energies and torsional barriers of alkanes, alcohols, ethers, and hemiacetals with an overall RMS deviation of 0.40 kcal/mol as compared to reference data. Densities of nineteen bulk liquids are predicted with an average error of 1.1%, and heats of vaporization are reproduced within 2.4% of experimental values. The force field is used to perform conformational analysis of smaller analogs of the macrocyclic polyketide drugs FK506 and/or Rapamycin. Structures that adopted low-energy conformations similar to that of bound natural drug are identified.

The details of the computer simulation approach to identify structures that adopted low-energy conformations similar to that of bound natural drug are further described in the Examples I II, and III, infra.

Biosynthesis of Candidate Compounds

Once candidate polyketide compounds are selected by the methods of the invention, the compounds are prepared by biosynthesis and other methods of efficiently producing these analogs using recombinant technology. The method applied is analogous to that described by Khosla et al. (U.S. Pat. No. 5, 712,146) but using the gene cluster for Rapamycin and/or FK506 (Katz, L. Chem. Rev. 1997, 2557-2575; Khosla, C. Chem. Rev. 1997, 2577-2590; Staunton, J.; Wilkinson, B. Chem. Rev. 1997, 2611-2629). The biosynthetic gene cluster for Rapamycin has been cloned and isolated previously (Schwecke et al., Proc. Natl. Acad. Sci. USA 1995, 92, 7839-7843).

For biosynthesis of Rapamycin and/or FK506 analogs, directed changes are made in the Rapamycin and/or FK506 PKS modules, i.e. deletion of modules, inactivation of individual domains within modules, domain replacement, site-directed mutagenesis of the domain, and/or gain-of-function mutagenesis within modules, to attain the desired PKS genes to catalyze synthesis of candidate compounds. A host-vector system, (hosts such as Streptomyces coelicolor CH999 (Fernandez-Moreno, M. A. et al., 1991, Cell 66, 769) or E. coli strain NM538 (Strategene); vectors such as pWE15 (Strategene) and, pUC118 or pUC18 (BRL)) is used to produce polyketide synthases (PKSs) which in turn catalyze the production of a variety of polyketides. In particular, genetically engineered host cells, which have their naturally occurring PKS genes substantially deleted, such as Streptomyces coelicolor CH999 (Fernandez-Moreno, M. A. et al., 1991, Cell 66, 769), are used. These host cells can be transformed with recombinant vectors, such as recombinant pWE15 (Strategene), containing genes encoding a variety of PKS gene clusters, for the production of active polyketides.

The genetically engineered cell expresses a polyketide synthase (PKS) gene cluster in its transformed state, where the host cell substantially lacks the entire native PKS gene cluster. The cell is transformed with a PKS gene cluster which encodes a PKS capable of catalyzing the synthesis of a polyketide; and with one or more control sequences operatively linked to the PKS gene cluster, so that the genes in the gene cluster are transcribed and translated in the genetically engineered cell. A population of engineered cells is then cultured under conditions so that the replacement PKS gene cluster present in the cells, is expressed. The novel polyketides produced are then isolated and characterized.

In addition, the modified or tethered Rapamycin and/or FK506 analogs as described in Example II, infra, containing the methylene chain replacement of the effector domain are obtained through chemobiosynthesis. Natural synthetic intermediates in the Rapamycin and/or FK506 pathway can be replaced with altered compounds, which are accepted at appropriate pathway points, and processed by the remaining Rapamycin and/or FK506 PKS enzymes. The modified analogs are synthesized by feeding thioesters, of varying chain lengths, to the bioengineered cells described above. The use of synthetic substrates in the presence of cell-free PKS enzymes, such as thioesters, can be used to enhance the diversity and complexity of the desired polyketides.

Verification of Bioactivity

After synthesis of the polyketide compounds of the invention, their activity is confirmed, for example using binding and cell culture assays. For Rapamycin and/or FK506 analogs, the activity is determined by measuring the neurotrophic actions of the candidate compounds. The compounds are evaluated for their effect on neuronal outgrowth and on neurite extension in cell culture and in sensory ganglia explants, and/or are tested to determine their augmentation of physical regrowth and functional recovery of damaged sciatic nerves. Preparing neuronal cell cultures, analysing of neurite lengths, binding and autoradiography are known (see Lyons et al. Proc Natl. Acad. Sci. USA, 1994, 92, 3191-3195; Steiner et al. Proc Natl. Acad. Sci. USA, 1997, 94, 2091-2024; Gold et al. J. Pharm. Exp. Ther. 1999, 289m 1202-1210). The compounds are also evaluated for their nonimmunosuppressive effects by determining the ability of these compounds to interact with FRAP or Calcineurin (Steiner, et al. Nature Med. 1997, 3, 421-428).

The polyketide compounds of the invention can also be confirmed for their anti-fungal or anti-tumor activity. For example, the ability of the compounds to reduce tumor mass or decrease the number of tumor cells can be evaluated. The antiproliferative activity of the polyketide compounds in various human tumor cells lines and tumor xenograft models can be used to demonstrate the efficacy of the compounds as anti-tumor agents (Alexandre J, et al Bull Cancer 1999, 86,808-811; Grewe M, et al. Cancer Res 1999, 59,3581-3587). Similarly, the ability of the compounds to kill fungal cells is determined (Hardwick J. S. et al Proc Natl Acad Sci U S A 1999, 96, 14866-14870; Cardenas M E, et al Clin Microbiol Rev 1999, 12, 583-611).

Uses of the Compounds of the Invention

The compounds of the invention may be used in methods capitalizing on the bioactivity of the compounds. For example, in the Examples described herein, analogs of Rapamycin and/or FK506 retaining only neuroregenerative properties and not immunosuppressive properties are obtained. These compounds are then used in methods for regenerating neurons in a subject by contacting the neurons with the compounds in a suitable carrier. The compounds of the invention may also be used as an anti-fungal, anti-tumor or anti-cancer agents.

Compounds of the invention can be derivatized to improve the bioavailability of the polyketide compound by rational drug design (See Silverman, R. B.; 1992. The Organic Chemistry of Drug Design and Drug Action, Academic Press, New York. For example, the use of prodrug strategies in drug design (Ibid., Chapter 8, 352-401) may be used to enhance the properties of the compounds and its ability to exerts it effects. The compounds may also be modified to affect properties such as solubility, absorption and distribution, stability, release time, and toxicity.

Accordingly, the compounds of the invention are useful in therapeutic and research contexts. In therapeutic applications, the compounds are utilized in a manner appropriate for neuroregenerative, antibiotic or anti-tumor therapy. The compositions may be suspended in a physiologically compatible pharmaceutical carrier, such as physiological saline, phosphate-buffered saline, or the like, to form a physiologically acceptable aqueous pharmaceutical composition for administration to a subject. Parenteral vehicles include sodium chloride solution, Ringer's dextrose, dextrose and sodium chloride and lactated Ringer's solution. Other substances may be added as desired, such as antimicrobials.

For therapeutic uses, the compounds of the invention can be formulated for a variety of modes of administration known in the art, including parenteral, for example intravenous, intraperitoneal, intramuscular, intradermal and epidermal, including subcutaneous and intradermal, oral, or applied to mucosal surfaces, e.g. by intranasal administration using inhalation of aerosol suspensions, and by implanting to muscle or other tissue in the subject. Suppositories and topical preparations are also contemplated. In addition, the compounds may be formulated in solid form and redissolved or suspended immediately prior to use. Lyophilized forms are also included. For oral administration, the compounds can be formulated into conventional oral administration forms such as capsules, tablets, and tonics. For topical administration, the compounds of the invention can be formulated into ointments, salves, gels, or creams as generally known in the art. Techniques and formulations generally may be found in Remmington's Pharmaceutical Sciences, Meade Publishing Co., Easton, Pa.

The most effective mode of administration and dosage regimen for the compounds of the invention depend on factors including the type and severity of disease, the subjects health, previous medical history, age, weight, height, sex and response to treatment, as well as the judgment of the treating physician. Therefore, the amount of compounds to be administered, as well as the number and timing of subsequent administrations, are determined by a medical professional conducting therapy based on the response of the individual subject. Initially, such parameters are readily determined by skilled practitioners using appropriate testing in animal models for safety and efficacy, and in human subjects during clinical trials of candidate therapeutic inhibitor formulations. For administration to mammals, and particularly humans, a typical daily dosage level of an active agent will be from 0.01 mg/kg to 10 mg/kg, typically around 1 mg/kg. The above dosages are exemplary of the average case. There can, of course, be individual instances where higher or lower dosage ranges are merited, and such are within the scope of this invention.

After administration, the efficacy of therapy using the compounds of the invention is assessed, for example, for accomplishing neuron regeneration, by an increase in the number of healthy neurons, or in the survival of the neurons (Gold, et al. Exp. Neurobiol. 1997, 147, 269-278; Gold et al. Soc. Neurosci. Abstr. 1997, 449:12). Preferably, non-invasive procedures are used to detect improvement in the patient's condition after administration of the compounds of the invention.

The following Examples are presented to demonstrate the methods and compounds of the present invention and to assist one of ordinary skill in making and using the same. The Examples are not intended in any way to otherwise limit the scope of the disclosure of the protection granted by Letters Patent granted hereon.

### EXAMPLE I

A Virtual Library of Novel Polyketide Compounds Generated by Successive Single Ketide Deletions from the Effector Domain of Rapamycin.

This Example describes the generation of a virtual Rapamycin library (by successive single ketide deletions from the effector domain of Rapamycin) and selection of modified Rapamycin compounds that would lack immunosuppressive activity and retain its neuroregenerative activity.

A comparison between the structures of several unnatural FKBP12-Rapamycin analog complexes (pdb entries 1fkg, 1fkh, and 1fki) (Holt et al., J. Am. Chem. Soc. 1993, 115:9925-9938) and the complexes of FKBP12-FK506 (pdb entry 1fkf) (Van Duyne et al., Science, 1991, 252:836-839) and FKBP12-Rapamycin (pdb entry 1fkb) (Van Duyne et al., J. Am. Chem. Soc. 1991, 113:743307434) was performed. This comparison showed that the conserved parts of the binding domain bind in an identical fashion in the complexes of FKBP12 with the unnatural Rapamycin analogs as with the polyketides. The crystal structures of the FKBP12-Rapamycin complex (Van Duyne et al., J. Am. Chem. Soc., 1991, 113:7433-7434) and the FKBP12-Rapamycin FRAP complex (Choi et al., Science, 1996, 273:239-242) were used as the basis for the molecular dynamics (MD) simulation studies in this work.

All MD simulations were performed using the Amber 5.0 computer program. (Case et al., AMBER; 50th Ed., University of California, San Francisco 1997). The FKBP12-Rapamycin analog (binary) complexes and FKBP12-Rapamycin analog-FRAP (ternary) complexes were placed in periodic water boxes that extended at least 9 A from all atoms in the complex. This resulted in box sizes of roughly 63×63×57 Å for the binary complexes and 74×84×60 Å for the ternary complexes.

The protein structures were protonated using the “protonate” utility in Amber, and subsequently equilibrated using a geometry optimization (200 steps of steepest descents and 9,800 steps of conjugate gradient optimization) and a 500 ps MD run of the protein-Rapamycin complexes. The equilibrated protein structures were used as the starting structures for the MD runs of the modified Rapamycin analogs. In all MD simulations, a constant dielectric of 1 was used and the non-bonded cutoff was set to 8 Å. 1-4 Van der Waals interactions were divided by 2.0, and 1-4 electrostatic interactions were divided by 1.2. Separate temperature scaling was used for the solvent and solute, the time constant for coupling to the external heat bath was 0.01 ps, the temperature was set to 300 K, the time step was set to 1 fs, and the non-bonded pair list was recalculated every 25 steps. During equilibration runs, bonds involving hydrogens were constrained using the SHAKE algorithm (Ryckaert et al., J. Comput. Phys. 1977, 23, 327), algorithm and interactions involving hydrogens were omitted. During the production portion of the dynamics runs, all interactions were calculated and the SHAKE algorithm was, therefore, not used. Subsequent runs consisted of a 1000 geometry optimization (200 steps steepest descent, 9,800 steps conjugate gradient), a 10 ps constant pressure equilibration run in which only waters were allowed to move, a 10 ps constant volume equilibration run using SHAKE, and a 100 ps constant volume production MD run (without geometry constraints or SHAKE) for each system examined. Each Rapamycin analog was studied unbound (in water) and bound to FKBP12. Additionally, the dynamics of some of the Rapamycin analogs were studied in ternary FKBP12-Rapamycin analog-FRAP complexes.

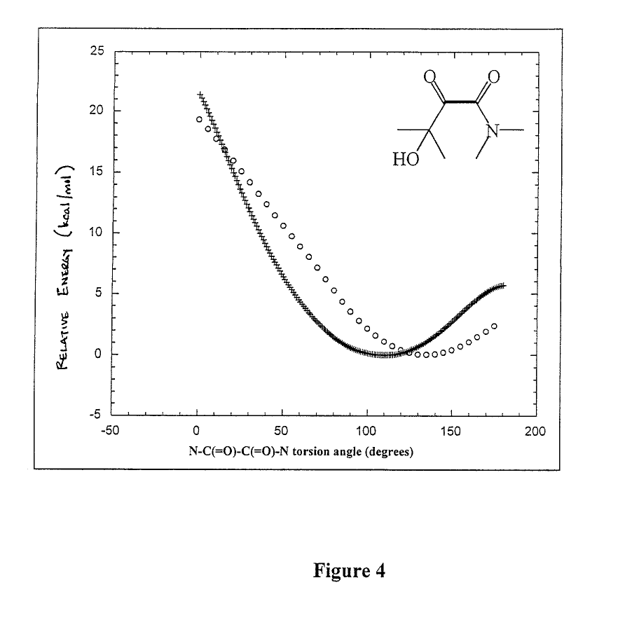

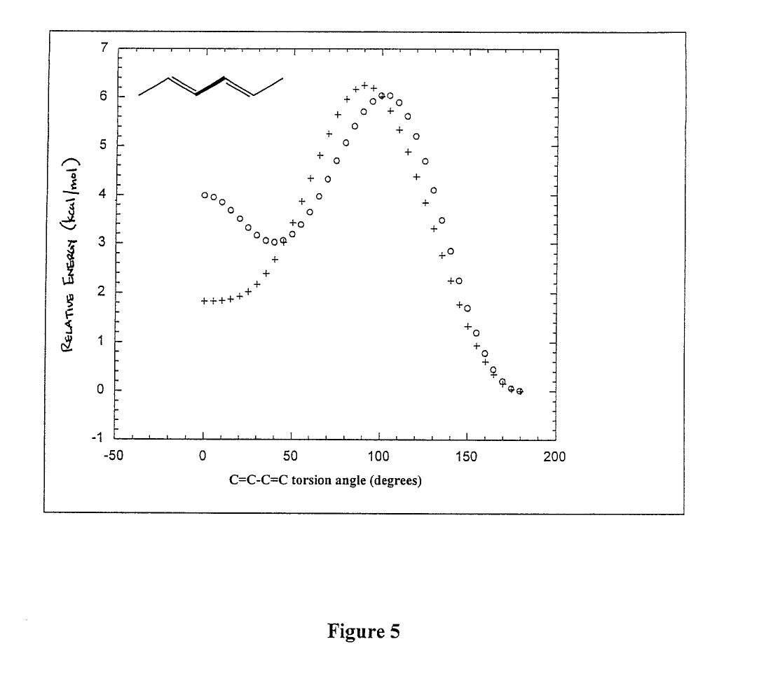

Charges for the Rapamycin analogs were calculated using averaged PM3 Mulliken charges for eight to ten distinct conformations of each Rapamycin analog, using the Ampac 6 program (Ampac, 6.0 Ed., Semichem, 7128 Summit, Shawnee, Kans. 66126, 1997). This was done after the calculation of standard RESP charges had proven prohibitively expensive (due to the large size and flexibility of the Rapamycin analogs, multiple conformations are required to get a reasonable ESP fit). The alpha-keto amide in the binding domain was assigned zero torsional parameters (i.e. the torsional energy profile was purely determined by electrostatic and van der Waals interactions) resulting in the energy profile shown in FIG. 4. The single bonds separating the adjacent double bonds in the binding domain were assigned parameters calculated by Hermone and Kuczera for retinal (FIG. 5). (Hermone and Kuczera, Biochem., 1998, 37:2843-2853). Using the parameters of Hermone and Kuczera reproduced the HF/6-31G(d) energy profile for rotation around the C3-C4 bond in 2,4-hexadiene when the bond was in a trans configuration, but the fit to the cis configuration was poor (FIG. 5). However, this was not a problem, since the adjacent double bonds cannot assume a cis configuration due to constraints placed by the macrocycle.

The binding of Rapamycin and the modified Rapamycin analogs was assessed based on the following criteria: (i) the mobilities of the protein and Rapamycin analogs during the MD simulations; (ii) the differences in the average solvent accessible surface area of the binding site of the protein bound to the different Rapamycin analogs; (iii) the formation of cavities between the protein and Rapamycin analogs during the MD simulations; and (iv) the difference in the interaction energies of the Rapamycin analogs with the proteins relative to the free Rapamycin analog and protein, scaled using the Linear Interaction Energy (LIE) method of Aqvist et al (Aqvist and Hansson, J. Phys. Chem. 1996 100:9512-9521 and Marelius and Hansson, J. Int. J. Quantum Chem. 1998 69:77-88). Each of the analysis methods used is described in more detail below.

The mobilities of the protein and Rapamycin analogs during the MD simulation were determined by estimating the average deviation of each atom from the average structure during the MD simulation. These can be compared to the crystal structure Debye-Weller B-factor (assuming that the B-factors are free from contributions from lattice effects and that the motions of atoms are isotropic) using the relationship <Δr2>^{1/2}=(3B/8π²)^{1/2} (McCammon et al. and Harvey, Dynamics of Proteins and Nucleic Acids, Cambridge University Press, Cambridge 1987). The comparison of the atom mobilities in the FKBP12-Rapamycin and FKBP12-Rapamycin-FRAP complexes with the Debye-Weller B-factors was used to verify that the secondary structure of the crystal structure remained intact during the MD simulation. The comparison of the mobilities of the protein bound to different Rapamycin analogs was used to indicate how the protein-Rapamycin analog binding was affected by the modifications to the Rapamycin analog.

The differences in the average solvent accessible surface area (SASA) of the protein binding site bound to the different Rapamycin analogs was used as a gauge of whether the modifications to the Rapamycin analogs affected the surface of FKBP12 as presented to FRAP. This was only relevant for the binary FKBP12-Rapamycin analog complexes. Solvent accessible surface areas were calculated at each point during the simulation using the program NACCESS. (Hubbard and Thornton, NACCESS, Department of Biochemistry and Molecular Biology, University College London, 1993). All residues that had at least one atom within 5 Å of Rapamycin in the binary complex of Rapamycin with FKBP12 were used in the SASA calculation. These were the amino acid residues Tyr26, Gly28, Phe36, Asp37, Phe46, Gln53, Glu54, Val55, Ile56, Arg57, Trp59, Tyr82, His87, Ile90, Ile91, Leu97, and Phe99.

The formation of cavities between the protein and Rapamycin analogs during the MD simulations was used to gauge how closely the Rapamycin analog filled into the binding sites on FKBP12 and FRAP. This measurement was mainly relevant for the ternary FKBP12-Rapamycin analog-FRAP complexes as a test of whether the binding to FRAP had been sufficiently affected by the modifications to Rapamycin to let water into the binding site. The volumes of gaps formed between the Rapamycin analog and protein were calculated at each step of the simulation using the program SURFNET. (Lakowski, J. Mol. Graph. 1995 13:18). The gaps were calculated using a 6×6×6 Å boundary around the atom range, a grid separation of 0.8 Å, an initial sphere size of 1 Å, a maximum sphere size of 4 Å, and a scaling factor of 1.0.

The difference in the interaction energies of the Rapamycin analogs with the proteins relative to the free Rapamycin analog and protein, scaled using the LIE method of Aqvist et al. (Aqvist and Hansson, J. Phys. Chem. 1996 100:9512-9521), were used to estimate the relative free energy of binding for Rapamycin and the modified Rapamycin analogs to FKBP12. The LIE binding free energy was also estimated for the ternary complexes, but it should mainly be taken as a qualitative measurement for the relative binding free energy in those cases. The LIE method is based on the assumption that, using molecular dynamics or Monte Carlo conformational simulations, the free energy of Rapamycin analog binding to a protein target can be expressed using the equation:

ΔG_{bind}=(αₚᵣₒₜ<Vₗ₋ₛ^{vdW}>ₚᵣₒₜ−α_{wat}<Vₗ₋ₛ^{vdW}>_{wat})+(βₚᵣₒₜ<Vₗ₋ₛ^{el}>ₚᵣₒₜ−β_{wat}<Vₗ₋ₛ^{el}>_{wat})+γ Eq. 1

where α-prot and α-wat are scaling factors for the average potential van der Waals energies (<Vvdw>), β-prot and β-wat are scaling factors for the average potential electrostatic energies (<Vel>), γ is an empirical constant to reproduce experimental free energies of binding, and the subscripts prot and wat refer to the FKBP12-Rapamycin analog complex in water and the free Rapamycin analog in water, respectively. Aqvist and coworkers fit Equation 1 to a large number of experimental binding constants and arrived at several possible combinations for the scaling factors α and β. Parameters were used which gave a good general fit in Aqvist's studies (α-prot and α-wat=0. 163, β-prot=0.348, β-wat=0.340, and γ=−1.89). Snapshots were taken at 1 ps intervals from the MD studies. The potential energies (Van der Waals and electrostatic) were calculated for the Rapamycin analog at each snapshot conformation using the “anal” program in the Amber suite of programs, and the average potential energies were used in calculating the free energies of binding from the LIE equation.

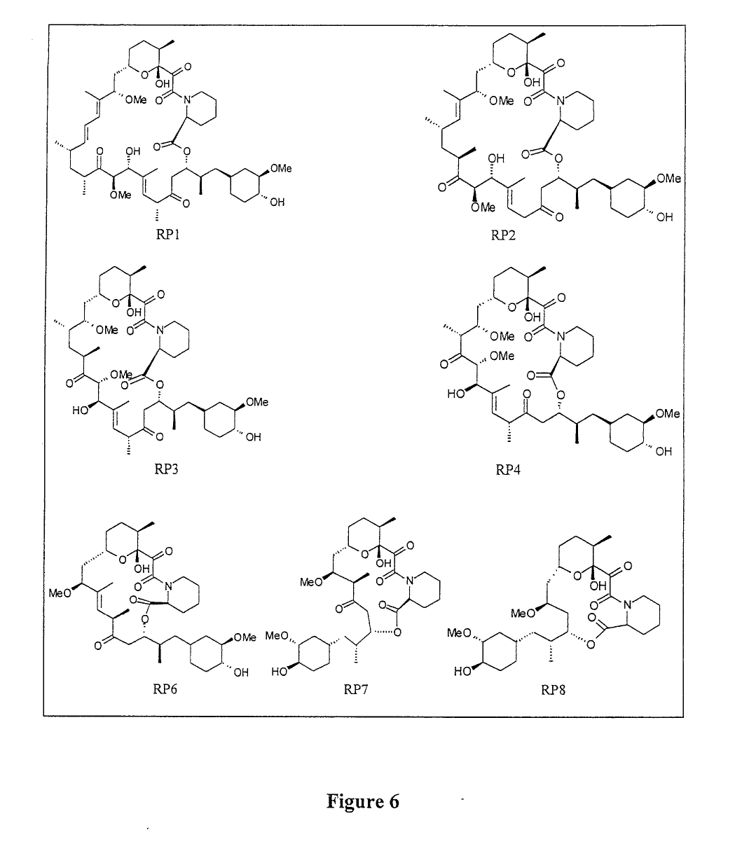

The Rapamycin analogs generated in this Example are shown in FIG. 6. All the analogs share the same binding domain but have effector domains that differ by the number of deletions from the effector domain of Rapamycin (FIG. 1). It is reasonable to expect larger differences in the interaction energies of the Rapamycin analogs with FRAP than with FKBP12, since all the differences focus on the part of Rapamycin associated with binding to FRAP. The changes in interactions between the Rapamycin analogs and the target proteins can be evaluated both from the interaction energies between the Rapamycin analogs and proteins and from the different dynamics of the Rapamycin analog-protein complexes.

Binary Rapamycin Analog-FKBP12 Complexes:

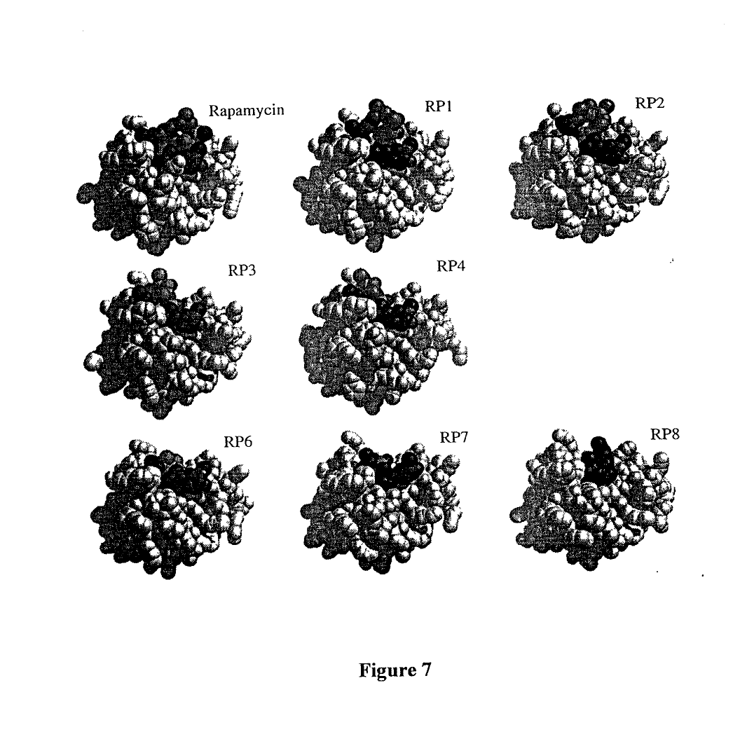

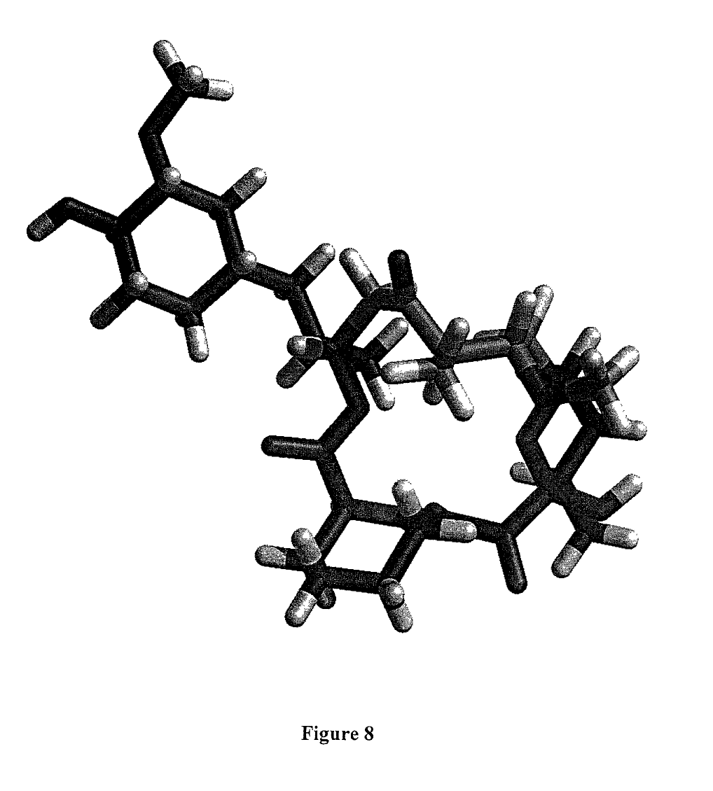

The ability of the FKBP12-Rapamycin complex to bind to FRAP is due, in large part, to the rigid nature of Rapamycin bound to FKBP12. When the binding domain of Rapamycin is bound to FKBP12, the three adjacent double bonds in the Rapamycin effector domain protrude rigidly away from the protein surface. The adjacent all-trans double bonded units in the effector domain form a rigid arm suitable for affixing to FRAP. FIG. 7 provides a sense of how the different Rapamycin analogs fit into the binding site on FKBP12. The position of the Rapamycin analog is generated by matching the binding domain of the Rapamycin analog with Rapamycin in the FKBP12-Rapamycin complex. The largest Rapamycin analogs protruded significantly from the protein surface, whereas the shortest ones were nearly completely engulfed by the protein. The effector domain of the smallest Rapamycin analogs barely protrudes from the surface of the protein, indicating a complete loss of ability to bind to the effector protein FRAP. FIG. 8 is a tube representation of the Rapamycin analog RP7, with the remaining portion of the effector domain shown in magenta. When ketides were removed from the effector domain of Rapamycin, the solvent accessible surface area (SASA) of the residues near the binding site stayed nearly constant during the first three deletions, decreased during the next three deletions, and then rose sharply (Table 1).

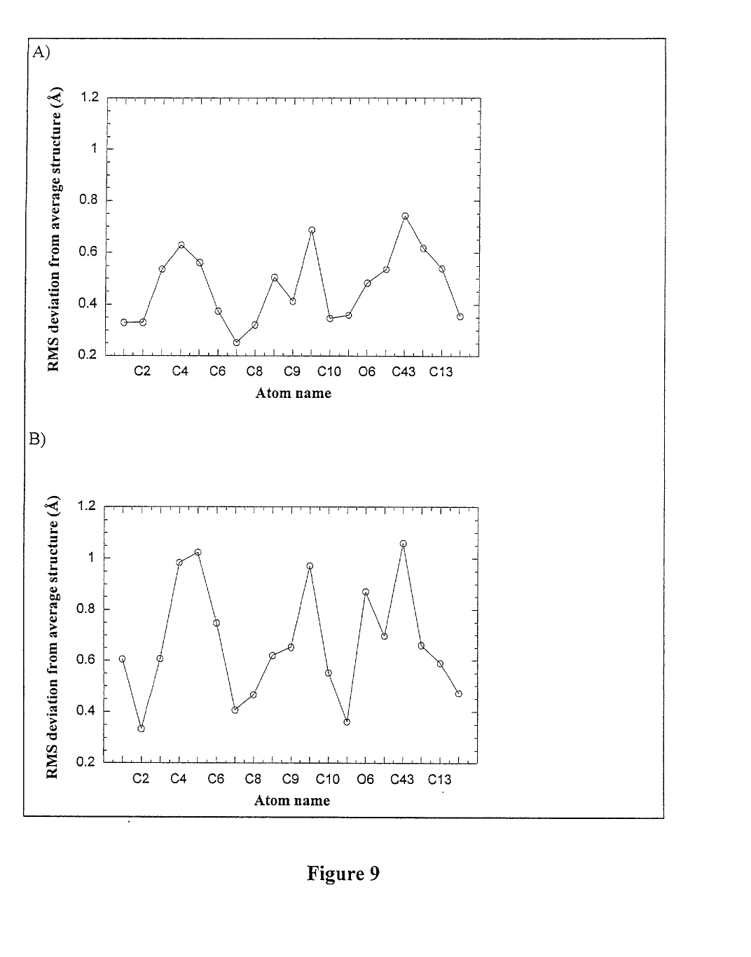

These changes in SASA reflect that the shorter effector domains of RP4, RP5, and RP6 do not protrude from the surface of FKBP12 as well as the effector domain of Rapamycin, but are instead in rather close contact with the protein. These close contacts between the protein and Rapamycin analogs are reflected in less favorable free energies of binding than were found for Rapamycin, RP2, and RP3. For the shortest Rapamycin analogs, RP7 and RP8, the effector domain is too short to protrude from the protein surface, and the Rapamycin analogs no longer favor the orientation required to bind to FKBP12. The poor fit of RP8 in the binding site of FKBP12 is reflected by the mobilities of the binding domains of the two Rapamycin analogs bound to FKBP12 (FIG. 9), which indicates that RP8 is making little or no favorable contacts with the binding site.

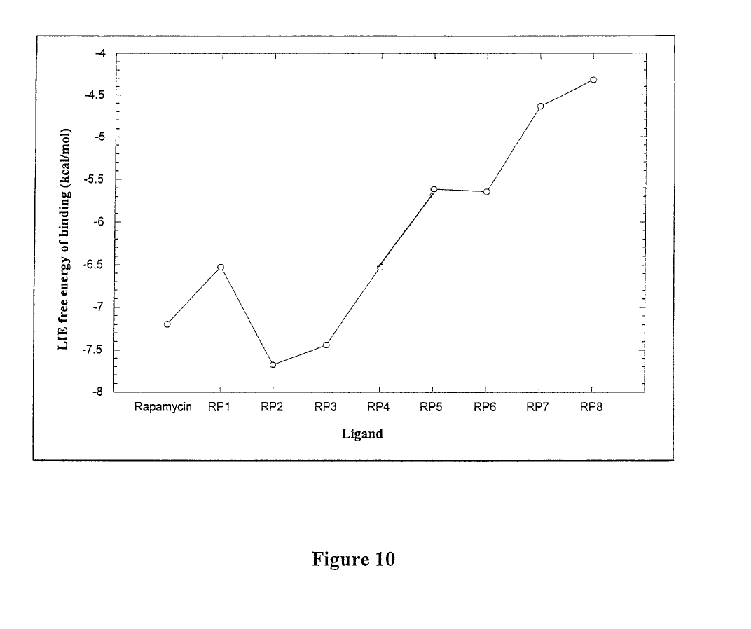

These findings are also reflected by the LIE free energies of binding, although RP1 does not fit the overall energy profile (FIG. 10). The free energy of binding for RP2 and RP3 is nearly identical to the free energy of binding calculated for Rapamycin, but the free energies of binding start getting less favorable as the effector domain is truncated further. These results, therefore, indicate that up to three ketide units can be removed from the effector domain of Rapamycin without adversely affecting the ability of the drug to bind to FKBP 12.

TABLE 1
The effects of sequentially deleting ketides from the effector domain
of Rapamycin on the formation of FKBP12-Rapamycin analog complexes.
The Rapamycin analogs are shown in FIG. 6. The average solvent
accessible surface area (SASA) is given along with its standard deviation
for each dynamics run.

| Rapamycin | | | |
|---|---|---|---|
| analog | SASA (C3) | RMS dev. | LIE (kcal/mol) |
| Rapamycin | 645 | 42 | −7.2 |
| RP1 | 612 | 33 | −6.5 |
| RP2 | 616 | 32 | −7.7 |
| RP3 | 621 | 30 | −7.4 |
| RP4 | 595 | 38 | −6.5 |
| RP5 | 584 | 28 | −5.6 |
| RP6 | 571 | 44 | −5.6 |
| RP7 | 630 | 45 | −4.6 |
| RP8 | 694 | 43 | −4.3 |

Ternary Rapamycin Analog-FKBP12 Complexes:

The interactions of Rapamycin and the modified Rapamycin analogs RP1, RP2, and RP3 bound in ternary complexes with the proteins FKBP12 and FRAP were evaluated using the same methods as were used for the binary FKBP12-Rapamycin analog complexes. The way that the modified Rapamycin analogs interact with FRAP was likely to be significantly different from the interactions between Rapamycin and FRAP. The formation of cavities between the Rapamycin analog and protein were, therefore, used as a qualitative measure of the effect that the changes in Rapamycin analog structure had on the FRAP binding. The LIE free energies of binding were also calculated.

As ketide units were removed from the effector domain of Rapamycin, increased cavities were formed between the Rapamycin analog and proteins. In the case of the smaller Rapamycin analogs, RP2 and RP3, water molecules from the simulation environment found their way into these cavities as the simulation progressed, indicating the loss of favorable interactions between FRAP and the modified effector domain of the Rapamycin analogs (Table 2). This same trend is reflected in the LIE free energies of binding, which grow steeply less favorable as the first double bonded ketide unit is removed and are identical for the modified Rapamycin analogs RP2 and RP3.

TABLE 2
The effects of sequentially deleting ketides from the effector domain
of Rapamycin on the formation of FKBP12-Rapamycin
analog-FRAP complexes. The Rapamycin analogs are shown in FIG. 6.
The average gap volume between the Rapamycin analog and proteins
is given along with its standard deviation for each dynamics run.

| Rapamycin | | | |
|---|---|---|---|
| analog | Gap (C3) | RMS dev. | LIE (kcal/mol) |
| Rapamycin | 904 | 147 | −10.7 |
| RP1 | 997 | 144 | −7.1 |
| RP2 | 1027 | 127 | −5.8 |
| RP3 | 1098 | 108 | −5.8 |

Conclusion:

The binding energies of Rapamycin and the modified Rapamycin analogs RP1 through RP8 are summarized in FIG. 10. These results indicate that several deletions (up to three ketide units) can be performed from the effector domain of Rapamycin without adversely affecting the ability of the Rapamycin analogs to bind to FKBP12. Indeed, the binding free energies calculated using the LIE method are predicted to become more favorable when one or more of the adjacent double bonded ketide units in the Rapamycin binding domain are deleted.

Without wishing to be bound by any one theory, this indicates that the role of the adjacent double bonds in binding to FRAP might be important enough for nature to accept the unfavorable effect this part of the Rapamycin analog has on the binding to FKBP12. The affinities of the modified Rapamycin analogs for binding to FKBP12 are predicted to drop off sharply when the deletions from the effector domain are numerous enough to bring substituents on the effector domain close to the surface of the protein (Rapamycin analogs RP5 and higher). The drop in binding affinities becomes even sharper when the deletions become severe enough to start directing the preferred conformation of the binding domain of the Rapamycin analog, as is seen for Rapamycin analogs RP7 and RP8.

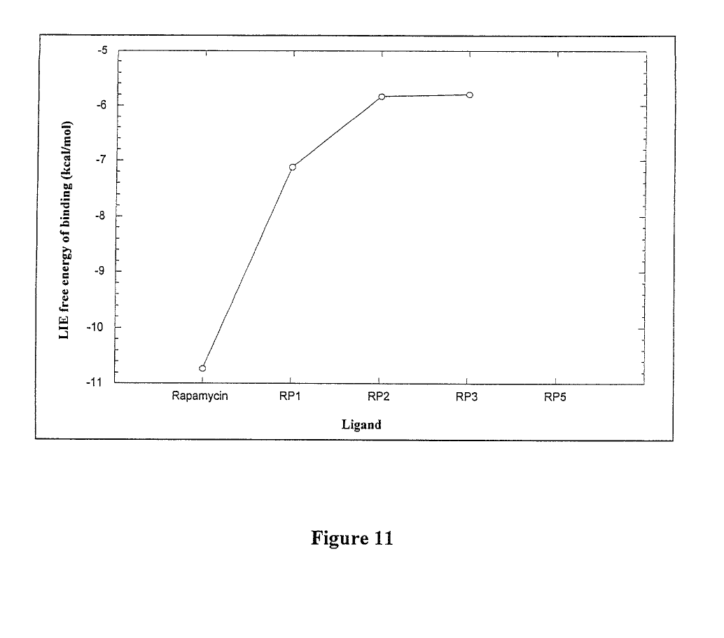

The binding energies for the Rapamycin and the modified Rapamycin analogs RP1, RP2, and RP3 in the ternary FKBP12-Rapamycin analog-FRAP complexes are summarized in FIG. 11. As can be seen from these results, the prediction of the MD simulations is that the favorable interactions between Rapamycin and FRAP are severely affected by the removal of one of the adjacent double bonded ketide units in the effector domain of Rapamycin. The interactions between the Rapamycin analog and FRAP are nearly eliminated with the removal of the second of the adjacent double bonds. An examination of FIG. 7 shows that the effector domains of the smaller Rapamycin analogs (notably RP6 through RP8) barely protrude from the protein surface, such that these Rapamycin analogs would not be able to associate with FRAP. This example shows a marked drop in the binding affinities of modified Rapamycin analogs to FKBP12 when the Rapamycin analog effector domain becomes smaller than five ketide units in length.

The best Rapamycin analogs found in the Example, RP2 and RP3, show that the ease of formation of the ternary FKBP12-Rapamycin-FRAP complex can be selectively varied through modifications to the effector domain of Rapamycin. It is also clear that the modifications performed to achieve these changes can be kept within the range of changes accessible by modifications to polyketide synthases. Thus, this Example demonstrates that the methods of the invention can be used to select novel polyketide compounds having desired properties having neuroregenerative but lacking immunosuppressive activities.

### EXAMPLE II

A Virtual Library of Novel Polyketide Compounds Generated by Substituting Different Ketide Units or Tethers to Replace the Effector Domain of Rapamycin.

This Example describes the generation of a virtual Rapamycin library (by substituting different ketide units or tethers to replace the effector domain), selection of modified Rapamycin compounds that would eliminate its immunosuppressive abilities and retain its neuroregenerative abilities, and are suitable for synthesis by modified polyketide synthesis.

In order to limit the number of conformations that had to be screened to determine conformational preferences, the cyclohexyl arm (FIG. 1) was not incorporated in the Rapamycin analogs. The crystal structure and MD studies of the FKBP12-Rapamycin complex reveal that the cyclohexyl arm points away from the macrocycle when the Rapamycin analog is bound to FKBP12. To compensate for the omission of this part of the Rapamycin analogs, binding conformations were selected based on maximal overlap between the methyl group chosen to represent the cyclohexyl arm and the adjacent carbon with the corresponding atoms in Rapamycin bound to FKBP12. The modified Rapamycin analogs examined are accessible through deletions from the biosynthetic gene cluster responsible for the Rapamycin PKS followed by modifications of the ketide units in the remaining chain.

The initial deletions examined resulted in three different compounds. The largest is the pentaketide Rapamycin analog I. The removal of the double bonded unit from I results in tetraketide Rapamycin analog II. The removal of a second ketide unit results in triketide Rapamycin analog III, which contains the shortest polyketide chain that can form the lactone bond needed to close the macrocycle.

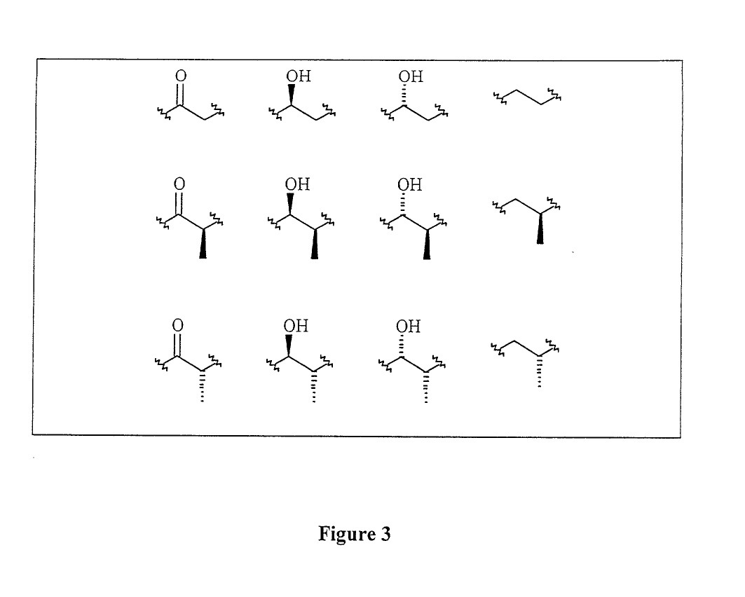

The conformational preference of the binding domain can be affected by substituents on the effector domain, particularly in Rapamycin analogs with shortened effector domains, as demonstrated in Example I. In order to adequately address these changes, all the analogs of I, II, and III obtained by ketide exchange with the ketide units in FIG. 10 were examined. The choice of the twelve ketide units in FIG. 3, out of the multitude of ketide units that can be generated by polyketide synthases, was based on the range of modifications currently accessible through engineered biosynthesis.

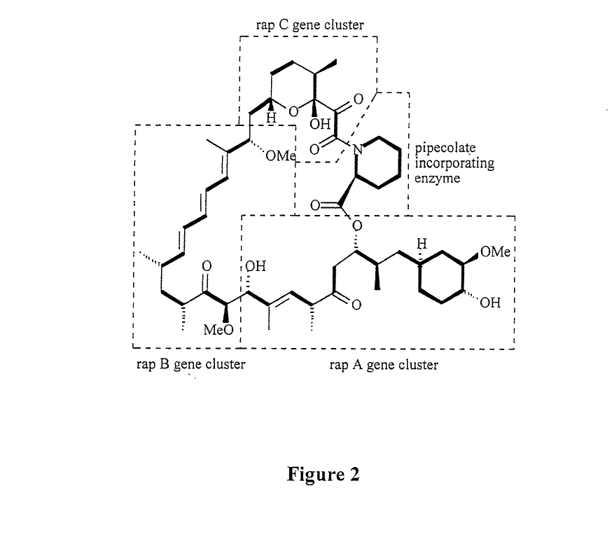

The following steps were carried out: (i) the ketide units encoded by the “rap B” frame in the biosynthetic gene cluster for Rapamycin, were removed, thereby creating a Rapamycin analog with a shortened effector domain (tether); (ii) zero, one, or two deletions from the “rap A” frame in the gene cluster (FIG. 2) were removed, which resulted in the three different tether lengths examined in this study; and (iii) finally each of the ketides in the tether was replaced with each of twelve ketides in FIG. 3 in a combinatorial fashion.

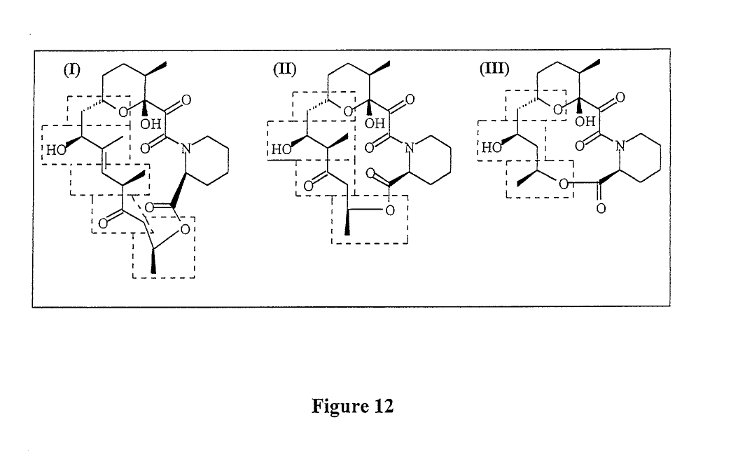

FIG. 12 provides examples of the substituted Rapamycin analog compounds examined. The dotted lines indicate the division of the tethers into individual ketide units, showing where substitutions were performed to generate novel tethers.

The generation of combinatorial libraries of novel polyketide Rapamycin analogs was accomplished as follows: The effector domain of Rapamycin was replaced with methylene chains corresponding to the lengths of the effector domains of I, II, and III to give three template Rapamycin analogs, pentaketide, tetraketide, and triketide, respectively, for subsequent substitutions. Each of the three template Rapamycin analogs was generated with the amide group in both the cis- and trans-conformations, and the resulting structures were used as starting points for stochastic searches (Saunders, J. Am. Chem. Soc. 109:3150 (1987); Saunders, J. Comp. Chem. 1989, 10:203) in the MM3(94) program (Goldstein et al., J. Phys. Org. Chem. 1996, 9:191-202). Each of the two conformations of the three template Rapamycin analogs was subjected to 5,000 Monte Carlo pushes, yielding pools of 600-1,500 conformations from each of the six runs. Conformations differing by at least 0.5 Å in RMS fit between all non-hydrogen atoms for the pentaketide and by at least 0.3 Å for the triketide and tetraketide were retained in the pools. This yielded an input pool of 349 different conformations for the pentaketide, 2,223 conformations for the tetraketide, and 1,416 different conformations for the triketide.

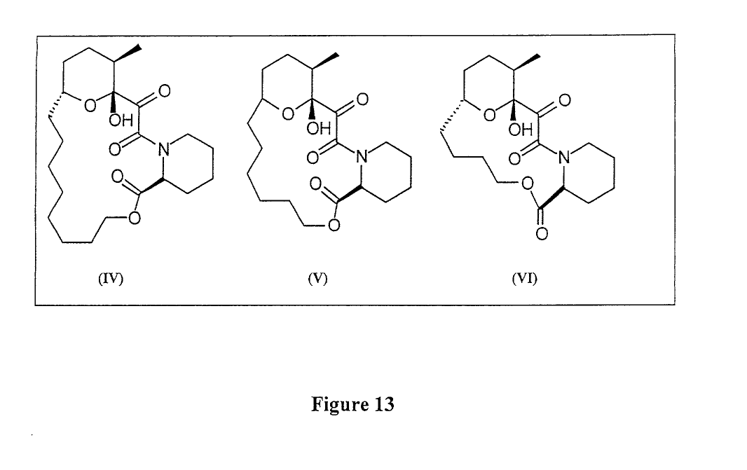

Each of the conformations in the input pools was used as a starting structure for each of the substitutions in FIG. 13. This yielded 101,952 input conformations for the triketides (1,416 different conformations and 72 possible substitutions), 1.9 million input conformations for the tetraketides (2,223 template conformations and 864 possible substitutions) and 32E6 million input conformations for the pentaketides (349 template conformations and 10,386 possible substitutions). A potential (single point) energy calculation was performed on each input conformation using MM3. The energy values from the single point calculations were used to calculate the probability for all the conformations of each substitution pattern using Boltzmann's equation for population distributions:

$${P}_{i} = \frac{{e}^{- β {E}_{i}}}{{\sum }_{j}^{N} {e}^{- β {E}_{j}}} \qquad ( 1 )$$

where Pi is the probability of finding the molecule in conformation i out of the N conformations considered, and β is 1/(kT), where k is the Boltzmann constant and T is the temperature. Since the geometries of the molecules remain fixed in the energy evaluations, conformations that hold the binding domain of the molecules in an orientation suitable for binding to FKBP12 can be identified from the template molecule pool. Based on those conformation searches, substitution patterns that favor suitable binding conformations can be rapidly identified from the calculated population densities. Initially, suitable binding conformations were defined as structures that had a least-squares fit of less than 0.5 Å from the atoms in the binding domain of the template molecule to the binding domain of Rapamycin bound to FKBP12. Good matches were subsequently identified through a visual inspection of the matched structures.

Molecules containing substitution patterns that were found to favor holding the binding domain of the molecules in an identical conformation as in Rapamycin and FK-506 bound to FKBP12 were docked in the binding site of FKBP12 and their binding evaluated using the same methods as were described in Example I above.

The structures of the pentaketide, tetraketide, and triketide Rapamycin analogs were created through replacing the effector domain with methylene chains corresponding to the lengths of the effector domain from Rapamycin (compounds I, II, and III, respectively) and are shown in FIG. 13. The Rapamycin analogs are obtained by removing the portion of Rapamycin encoded by the “rap B” part, and zero, one, or two of the ketides encoded by the “rap A” part of the biosynthetic gene cluster responsible for generating Rapamycin (FIG. 2). Additionally, combinatorial combinations of the ketide units shown in FIG. 3 were generated by substitutions in the effector domains I, II, and III. Each Rapamycin analog pool contained 6×12(n−2) different substitutions, where “n” is the number of ketide units in the Rapamycin analog tether (n=3 for the triketide, n=4 for the tetraketide, and n=5 for the pentaketide). The conformational preference of each Rapamycin analog in the pools was evaluated, and the binding affinities of the Rapamycin analogs resulting from the deletions and the highest scoring candidates from the pools with FKBP12 were evaluated using molecular dynamics (MD) simulations.

Triketide:

The triketide Rapamycin analog VI contains the shortest modified effector domain that can link the two ends of the binding domain while still retaining a conformation similar to that of Rapamycin with FKBP12. Previous results for modified Rapamycin Rapamycin analogs indicated that the binding affinities of Rapamycin analogs of this length to FKBP12 might be adversely affected by conformational constraints placed by the short effector domain.

The stochastic conformational search on VI revealed that the Rapamycin analog couldn't assume the binding orientation of Rapamycin. The lowest energy conformation that closely resembles the required binding orientation has a least squares fit of 0.36 Å for the picolinyl and tetrahydrofuranyl rings. The two conformations exhibit a close fit between the picolinyl rings of the two Rapamycin analogs (0.06 Å), but the tetrahydrofuranyl ring in III is rotated 35 degrees relative to Rapamycin. Acceptable binding conformations found for III (less than 0.5 Å least squares fit to the Rapamycin binding conformation) have a combined probability of 1.7%.

Compound III was docked in the binding domain of FKBP12 and an MD run was performed on the solvated complex. The average gap between the Rapamycin analog and protein is 207±44 Å³, which is indicative of the fact that compound III covers a very small portion of the protein. This is also evidenced by the SASA of the residues near the binding pocket, which is 727±83 Å², comparable the 989 Å² SASA of these residues in the absence of a Rapamycin analog. The LIE binding energy of III to FKBP12 is −3.5 kcal/mol, 3.1 kcal/mol less favorable than was found for Rapamycin.

Tetraketide:

The tetraketide Rapamycin analog V has the same effector domain length as the modified Rapamycin Rapamycin analog RP7 examined in Example I. The results for RP7 indicated that the conformational preference of the binding domain might be influenced by the substitutions on the effector domain, although the effects were not as readily apparent as for RP8, the counterpart to the triketide tether.

The tetraketide tether is flexible enough that the binding domain can readily assume the binding conformation of Rapamycin. The criteria for a suitable binding candidate were, therefore, reduced to 0.3 Å least squares fit between the binding domains of the Rapamycin analogs, rather than the 0.5 Å cutoff applied to the triketide Rapamycin analogs. The stochastic conformational search on II revealed a 1.9% probability of residing in any conformation with a better than 0.3 Å least squares fit to the binding domain of Rapamycin. The least squares fit between the binding domains is 0.18 Å.

Compound II was docked in the binding pocket of FKBP12 and a molecular dynamics run was performed on the solvated complex. The average gap between the Rapamycin analog and FKBP12 was 338±79 Å³. The SASA of the FKBP12 residues close to the binding pocket was 757±37 Å², and the LIE free energy of binding for the complex formation was −3.4 kcal/mol, which is nearly identical (0.1 kcal/mol less favorable) to the binding free energy that was calculated for III.

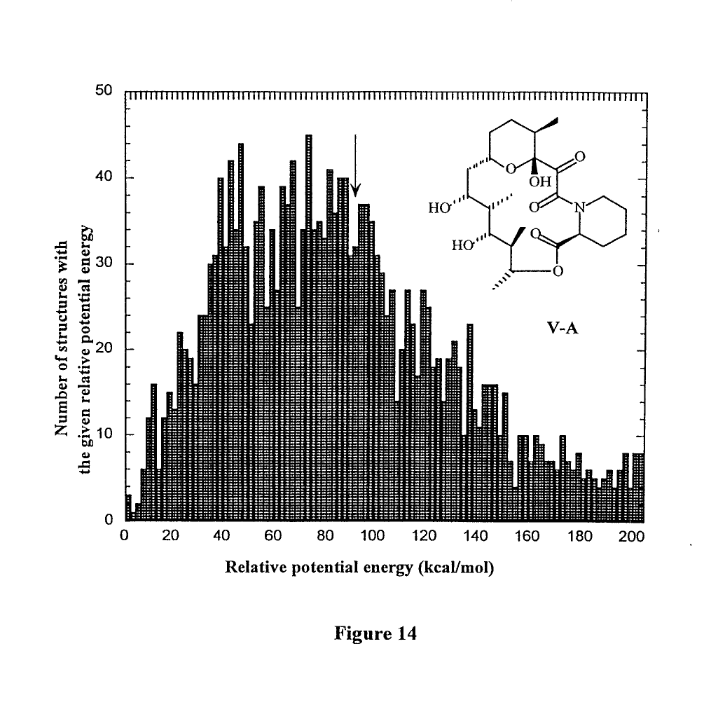

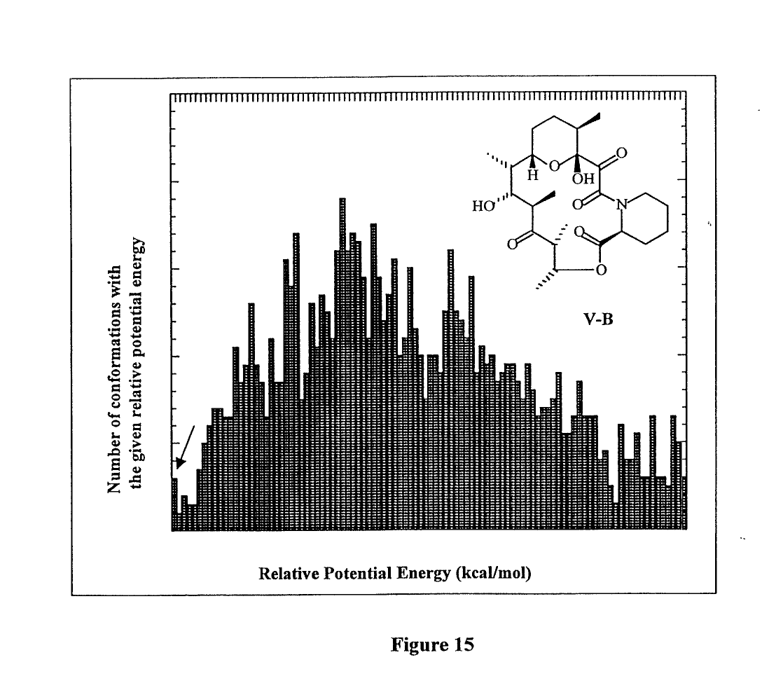

The conformational searches on the 864 different substitution patterns examined for the generated tetraketide Rapamycin analogs showed that the substitution patterns can have a marked effect on the conformational preference of the binding domain in much the same way as was observed for the triketide Rapamycin analogs. Bar plots for two representative energy distributions are shown in FIGS. 14 and 15 (the lowest energy binding structures are indicated by arrows on the Figure). The bar plot in FIG. 14 is for compound V-A. Compound V-A strongly disfavors any of the input conformations that has a better than 0.5 Å least squares fit to the Rapamycin binding domain. The bar plot in FIG. 15 is for compound V-B, which was predicted by the single point energy calculations to have a 100% probability of existing in a conformation that had a least squares fit to the binding conformation of Rapamycin of 0.17 Å. A stochastic conformational search of compound V-B predicted that the Rapamycin analog had a 3.9% probability of existing in the binding conformation.

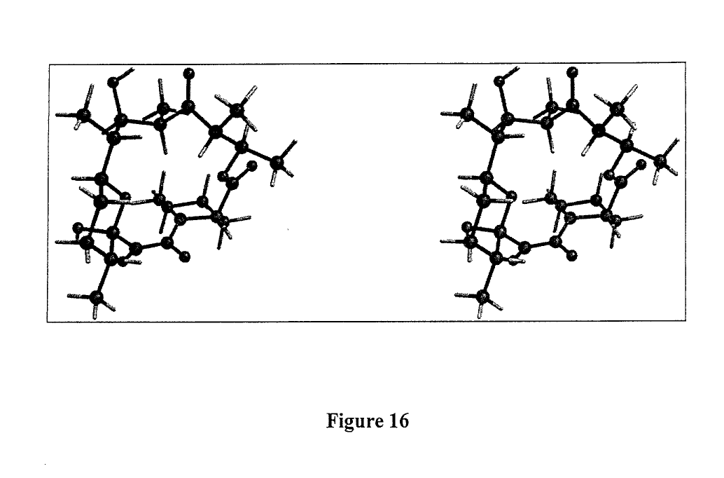

This low probability for the binding conformation comes about because there is another very low energy conformation (roughly 2 kcal/mol lower in energy than any other conformation found). This low energy conformation is characterized by an intramolecular hydrogen bond between the proton on the alcohol substituent and the amide carbonyl oxygen in the binding domain. Such a hydrogen bond would exist in the gas phase but not in aqueous solution. Thus, this conformation was excluded from the probability calculations. When this low energy conformation is omitted, the probability of assuming a suitable binding conformation is 26%. FIG. 16 is a stereo view of the favored binding conformation of V-B. The substituents along the effector domain are arranged such that no unfavorable steric contacts arise between them, whereas rotations out of the desired binding conformation bring the substituents into close proximity of one another.

Compound V-B was docked in the binding pocket of FKBP12 and a molecular dynamics run was performed on the resulting complex. The average gap between the Rapamycin analog and FKBP12 during the MD run is 486+/−138 A³, roughly half the gap that was observed for Rapamycin. The SASA of the residues near the binding pocket was 751+/−38 A2. The calculated free energy of binding to FKBP12 was −7.1 kcal/mol, which is 0.5 kcal/mol more favorable than was found for Rapamycin and 3.7 kcal/mol more favorable than the interaction energy that was calculated for II.

Pentaketide:

The pentaketide Rapamycin analog IV possesses a tether length equivalent to the modified Rapamycin Rapamycin analog RP6 of our previous example. The results for RP6 did not conclusively indicate whether the substituents on the Rapamycin analog effector domain had a determining effect on the conformational preference of the binding domain.

Compound I has a 0.3% probability of existing in a conformation whose least squares fit to Rapamycin was better than 0.3 Å. The lowest energy structures of I have the amide in the opposite configuration from that found in the crystal structure of Rapamycin bound to FKBP12. Compound I was docked in the binding pocket of FKBP12 and a molecular dynamics simulation was run on the solvated complex. The average gap between the Rapamycin analog and FKBP12 during the simulation was 363±88 Å³, which is slightly smaller than was found for the tetraketide II. The SASA of the residues near the FKBP12 binding site is 727=±83 Å², which is nearly identical to the SASA of the triketide III. This minor effect of the Rapamycin analog tether on solvent accessibility to the protein comes about because of the rigid nature of the double bond in the tether. The free energy of binding I to FKBP12, calculated using the LIE method, was −4.0 kcal/mol, which is 2.6 kcal/mol less favorable than for Rapamycin and only 0.5 kcal/mol more favorable than for III.

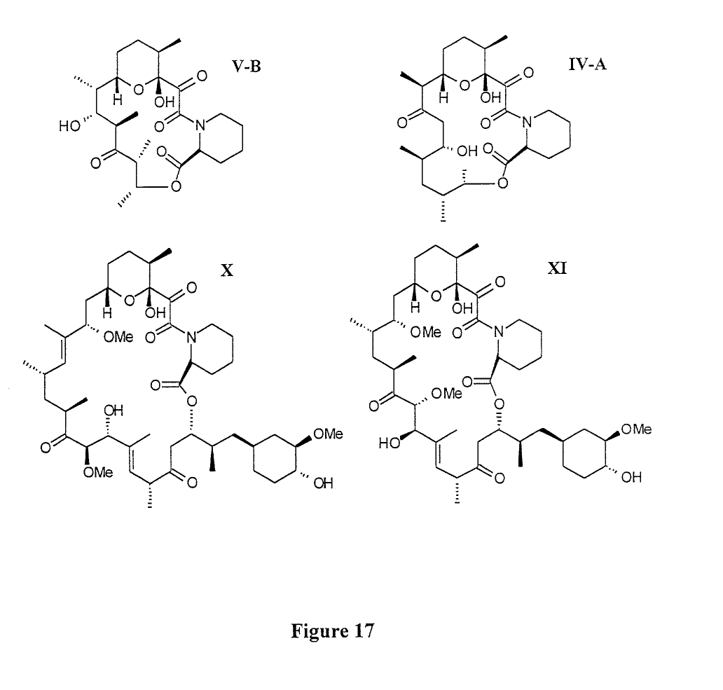

A conformational study was performed on the 10,386 substitutions for a pentaketide tether. Since the large number of possible substitutions and the flexibility of the template molecule makes it unfeasible to sample conformations throughout the conformational space of the template molecule, the pool of input conformations was more sparsely populated than the input conformations used earlier. The 349 template conformations examined for the Rapamycin analogs were not biased toward any specific orientation and contained roughly even numbers of the two amide conformations. Due to the limited number of input conformations and large number of possible substituents, 995 Rapamycin analogs from the pool were predicted to have over 50% probability of existing in a conformation suitable for binding to FKBP12. The substitutions that most frequently appeared in feasible candidates were combined to build the Rapamycin analog IV-A shown in FIG. 17. Compound IV-A was docked in the binding pocket of FKBP12 and MD studies were run on the resulting complex.

The average gap between IV-A and FKBP12 in the MD simulation was 296±50 Å³, slightly smaller than was observed for I. The average SASA of the residues near the binding pocket was smaller than was observed for I, 669±76 Å². This was because the preferred conformation of the Rapamycin analog in the MD simulation was with the tether oriented toward the protein surface. As was observed for compound V-B, rotations out of the desired binding conformation bring the substituents on the effector domain into close proximity of one another. The LIE free energy of binding for IV-A to FKBP12 was −7.0 kcal/mol, which is 0.4 kcal/mol better than was observed for Rapamycin and 3.0 kcal/mol more favorable than the free energy of binding of I.

Conclusion:

This Example reveals that the binding affinity of modified Rapamycin analogs to the target protein FKBP12 can be modulated by varying the substituents on the effector domain of the novel polyketide. All the structures in this example were designed such that they could not bind to FRAP. Good agreement is seen between the probability that a modified Rapamycin analog will exist in the required binding conformation and the free energies of binding to the protein target calculated using the LIE method.

These results show that Rapamycin analogs with effector domains corresponding to a tetraketide or pentaketide chain can assume the conformation required to bind to FKBP12, but Rapamycin analogs with triketide effector domains are unable to assume the required conformation. Thus, the best FKBP12 Rapamycin analogs found using this method had calculated free energies of binding that were marginally more favorable than the free energy of binding for Rapamycin, and greatly more favorable than for the Rapamycin analogs obtained by simple deletions from the Rapamycin effector domain. Thus the tethered analogs are predicted to be the best candidates having the desired properties. The design of the compounds presented is such that they are attainable through biosynthetic strategies, which include genetic modifications to the polyketide synthase (PKS) enzyme responsible for generating Rapamycin.

The structures of the four best candidates from the two studies in this Example are shown in FIG. 17. The first two compounds, structures V-B and IV-A appear to be the best candidates. These compounds were designed to be accessible by genetic modifications to the Rapamycin PKS in which units from the effector domain are deleted or replaced. The last two compounds, structures X and XI, were the best candidates from the preceding example. These compounds were designed to be accessible deletions of modules from the Rapamycin PKS. All the compounds in FIG. 17 have the same or better predicted binding affinity to FKBP12 as Rapamycin, using the LIE method.

In summary, this example shows that the ease of formation of the ternary FKBP12-Rapamycin-FRAP Rapamycin analog can be selectively varied through modifications to the effector domain of Rapamycin, and that the modifications performed to achieve these changes can be kept within the scope accessible by biosynthetic and chemobiosynthetic techniques.

### EXAMPLE III

Parameterization of OPLS-AA Force Field for the Conformational Analysis of Macrocyclic Polyketides

This Example describes a parameterized Force Field conformational analysis approach to obtain smaller polyketide analogs of macrocyclic polyketide drug FK506 that adopt low-energy conformation similar to that of the bound native molecule.

In order to accurately predict relative binding free energies of polyketide analogs of the macrocyclic polyketide drug FK506 (FIG. 18) so as to target proteins via simulation methods, the OPLS-AA force field was chosen for the conformational analysis (Jorgensen, W. L. et al., J Am Chem Soc 1996, 118:11225-11236). In this approach, the energy of a molecular system is derived as the sum of bond stretching, bond bending, torsional, and nonbonded terms. The bond stretching and bending parameters come mostly from Weiner's 1986 AMBER force field (Weiner, S. J. et al., J Am Chem Soc 1984, 106:765-784) with the exception of alkane parameters that have been adopted from CHARMM. The atomic charges and Lennard-Jones parameters have been fitted to reproduce the densities, heats of vaporization, and free energies of hydration of a wide range of organic compounds (Jorgensen, W. L.; et al., J Am Chem Soc 1996, 118:11225-11236; McDonald, N. A. et al., J Phys Chem B 1998, 102: 8049-8059; Rizzo, R. C. et al., J Am Chem Soc 1999, 121: 4827-4836; Price, M. L. et al., J Comput Chem 2001, 22:1340-1352). In some instances, fitting to electrostatic potential has been used to assign atomic charges (Jorgensen, W. L.; McDonald, N. A. J Mol Struct (THEOCHEM) 1998, 424, 145-155). Also, ab initio calculations of intermolecular energies of dimers or molecular hydrates in the gas phase have been used for determination of nonbonded parameters (McDonald, N. A. et al., J Phys Chem B 1998, 102: 8049-8059; Jorgensen, W. L. et al., J Mol Struct (THEOCHEM) 1998, 424:145-155). Most torsional parameters in the OPLS-AA force field were derived based on HF/6-31G* calculations on model compounds (Price, M. L. et al., J Comput Chem 2001, 22:1340-1352; Jorgensen, W. L. et al., J Mol Struct (THEOCHEM) 1998, 424:145-155). More recently, calculations at DFT or MP2 level of theory have been employed to parameterize carbohydrates (Damm, W. et al., J Comput Chem 1997, 18: 1955-1970), perfluoroalkanes (Watkins, E. K. et al., J Phys Chem A 2001, 105, 4118-4125), and peptides (Kaminski, G. A. et al., J Phys Chem B 2001, 105: 6474-6487).

In the course of calculating the conformations of polyketides, it became clear that in several cases the torsional parameters are lacking or are not sufficiently accurate in order to predict the correct order of conformer stabilities or the magnitude of torsional barriers. The HF/6-31 G* model, which was used for deriving torsional parameters in OPLS-AA, may not be adequate for describing torsional profiles of larger flexible molecules where dispersion interactions become important. Electron correlation effects tend to stabilize folded conformers over extended forms, for example, the trans-gauche energy difference in butane is predicted to be 1.01 kcal/mol from the HF/6-31G* calculations while the recently determined Born-Oppenheimer limit is 0.62 kcal/mol (Allinger, N. L. et al., J Chem Phys 1997, 106: 5143-5150). The weak performance of HF/6-31G* and also MP2/6-31G* methods for conformational energies has been noted previously. For example, Halgren has reported that HF/6-31G* calculations deviate on average 0.72 kcal/mol from the experimental values when conformer energies for a wide range of organic compounds are compared (Halgren, T. A., J Comput Chem 1999, 20:730-748). On the other hand, MP2 calculations with the cc-pVTZ(-f) basis set were shown to yield conformational energy differences with RMSD versus experiment of 0.35 kcal/mol (Halgren, T. A., J Comput Chem 1999, 20:730-748).

To remedy this weakness of the OPLS-AA force field, high-level ab initio conformational analysis of several alkanes, alcohols, ethers, hemiacetals, esters, and dicarbonyl compounds was carried out. The augmented cc-pVTZ basis set for conformational analysis was used because the diffuse functions may be important for conformational energies in molecules with lone electron pairs (Tozer, D. J. Chem Phys Lett 1999, 308:160-164). It was subsequently found that parameterization of the intramolecular portion of the force field alone was not sufficient, and thus some nonbonded parameters were modified as well. The new nonbonded parameters were tested and refined by performing Monte Carlo simulations of bulk organic compounds. Using these paramenter, an overall improvement was observed in predicting both the bulk properties and conformational energies. With the current parameterization, the OPLS-AA force field can be successfully used for conformational analysis of complex organic molecules containing the hydroxy-, alkoxy-, ester-, and dicarbonyl substituents in saturated aliphatic chains or rings.

Ab Initio Calculations.

Ab initio calculations using the program Gaussian98 (Frisch, M. J.; Trucks, G. W.; Schlegel, H. B.; Scuseria, G. E.; Robb, M. A.; Cheeseman, J. R.; Zakrzewski, V. G.; Montgomery, J. A. J.; Stratmann, R. E.; Burant, J. C.; Dapprich, S.; Millam, J. M.; Daniels, A. D.; Kudin, K. N.; Strain, M. C.; Farkas, O.; Tomasi, J.; Barone, V.; Cossi, M.; Cammi, R.; Mennucci, B.; Pomelli, C.; Adamo, C.; Clifford, S.; Ochterski, J.; Petersson, G. A.; Ayala, P. Y.; Cui, Q.; Morokuma, K.; Malick, D. K.; Rabuck, A. D.; Raghavachari, K.; Foresman, J. B.; Cioslowski, J.; Ortiz, J. V.; Stefanov, B. B.; Liu, G.; Liashenko, A.; Piskorz, P.; Komaromi, I.; Gomperts, R.; Martin, R. L.; Fox, D. J.; Keith, T.; Al-Laham, M. A.; Peng, C. Y.; Nanayakkara, A.; Gonzalez, C.; Challacombe, M.; Gill, P. M. W.; Johnson, B.; Chen, W.; Wong, M. W.; Andres, J. L.; Gonzalez, C.; Head-Gordon, M.; Replogle, E. S.; Pople, J. A. Gaussian 98; Gaussian, Inc.: Pittsburgh Pa., 1998) were performed to locate minima and saddle points in the conformational energy surface of ethane, propane, isobutane, pentane, isopentane, neopentane, 2,2-dimethylbutane, methanol, ethanol, 1-propanol, 2-propanol, sec-butanol, tert-butanol, cyclohexanol, ethyl methyl ether (EME), diethyl ether (DEE), methyl propyl ether (MPE), isopropyl methyl ether (IME), oxane, methoxymethanol (MMeOH), methoxyethanol (MEtOH), tetrahydropyran-2-ol (THPOH), 3-hydroxy-2-oxopropanal (HOP), 3-methoxy-2-oxopropanal (MOP), 3-hydroxy-3-methoxy-2-oxopropanal (HMOP), 2-glyoxoyl-tetrahydropyran (GTHP), 2-glyoxoyl-tetrahydropyran-2-ol (GTHPO), methyl formate, methyl acetate, methyl propionate, ethyl formate, propyl formate, isopropyl formate, and a pipecolinyl ester (MPIP). The structures and the corresponding abbreviations for some of these molecules are shown in FIG. 19. For molecules with complex potential energy surfaces, torsional profiles were generated by varying the torsional angle of interest in 20-40 degree increments and minimizing all other degrees of freedom. All molecules were initially optimized at the MP2/aug-cc-pVDZ level and single point energies were evaluated at the MP2/aug-cc-pVTZ level (hereafter abbreviated as MP2/TZ//DZ) except THPOH, for which only MP2/aug-cc-pVDZ energies were determined. Two larger molecules, GTHP and GTHPO, were studied at the MP2/aug-cc-pVDZ//MP2/6-31+G(d,p) level. The torsional profile for the pipecolinyl ester was determined at the MP2/6-31+G(d,p) level. A comparison with available experimental data suggested that the MP2/TZ//DZ approach yields an accurate description of the conformational energy surface but bond lengths are systematically overestimated. To test if this overestimation leads to errors in conformational energies, low energy conformers of ethane, propane, isobutane, methanol, ethanol, 1-propanol, 2-propanol, EME, methoxymethanol, HOP, methyl formate, methyl acetate, and ethyl formate were reoptimized at the MP2/aug-cc-pVTZ level. It has been shown previously that this level of theory predicts bond lengths with an accuracy of 0.006 Å(He, Y. and Cremer, D. J Phys Chem A 2000, 104, 7679-7688; Helgaker, T. et al., J Chem Phys 1997, 106, 6430-6440). The optimization with the triple-zeta basis set did not change conformational energies significantly. In some cases, the conformational energies evaluated at the MP2/aug-cc-pVDZ level were found to be significantly different compared to the MP2/TZ//DZ results (see Table 3). We also observed that the relative energies for conformers of GTHP and GTHPO showed differences up to 0.6 kcal/mol when MP2/6-31+G(d,p) optimized energies were compared to the MP2/aug-cc-pVDZ//MP2/6-31+G(d,p) results. For this reason GTHP and GTHPO data were excluded from the statistical analysis.

Force Field Parameterization.

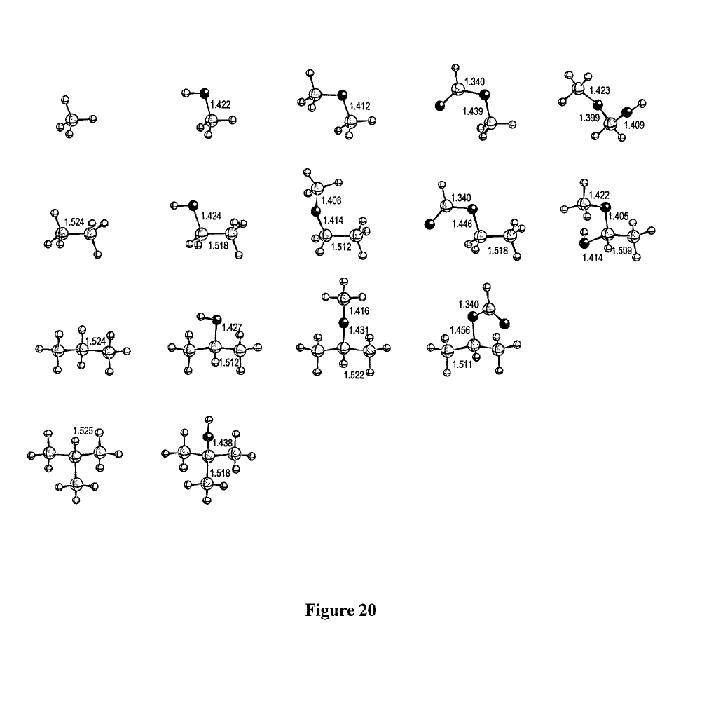

The ab initio data were used to reparameterize the OPLS-AA(2,2) force field to yield a better description of oxygen-containing organic compounds. The parameters were refined starting with bond stretching and bending parameters followed by optimization of nonbonded parameters and torsional force constants. All molecular mechanics calculations were performed with the program BOSS42 (Jorgensen, W. L. BOSS Version 4.2; Yale University: New Haven, Conn. 2000). The equilibrium bond lengths and angles were obtained by fitting the OPLS-minimized structures to MP2/aug-cc-pVTZ structures of molecules shown in FIG. 20.

Determination of atomic charges for alcohols was based on fitting the molecular electrostatic potential to atom-centered point charges using the constraint that functional units (such as CH₃ in ethanol) carry a net zero charge. The electrostatic potential was obtained from MP2/aug-cc-pVDZ densities using the Merz-Singh-Kollman scheme with 12 layers in Gaussian98. (Frisch, M. J.; Trucks, G. W.; Schlegel, H. B.; Scuseria, G. E.; Robb, M. A.; Cheeseman, J. R.; Zakrzewski, V. G.; Montgomery, J. A. J.; Stratmann, R. E.; Burant, J. C.; Dapprich, S.; Millam, J. M.; Daniels, A. D.; Kudin, K. N.; Strain, M. C.; Farkas, O.; Tomasi, J.; Barone, V.; Cossi, M.; Cammi, R.; Mennucci, B.; Pomelli, C.; Adamo, C.; Clifford, S.; Ochterski, J.; Petersson, G. A.; Ayala, P. Y.; Cui, Q.; Morokuma, K.; Malick, D. K.; Rabuck, A. D.; Raghavachari, K.; Foresman, J. B.; Cioslowski, J.; Ortiz, J. V.; Stefanov, B. B.; Liu, G.; Liashenko, A.; Piskorz, P.; Komaromi, I.; Gomperts, R.; Martin, R. L.; Fox, D. J.; Keith, T.; Al-Laham, M. A.; Peng, C. Y.; Nanayakkara, A.; Gonzalez, C.; Challacombe, M.; Gill, P. M. W.; Johnson, B.; Chen, W.; Wong, M. W.; Andres, J. L.; Gonzalez, C.; Head-Gordon, M.; Replogle, E. S.; Pople, J. A. Gaussian 98; Gaussian, Inc.: Pittsburgh Pa., 1998; Singh, U. C. and Kollman, P. A. J Comput Chem 1984, 5, 129-145). Potentials for several similar molecules or many conformations of one molecule were fitted simultaneously with the program RESP (Bayly, C. I. et al., J Phys Chem 1993, 97, 10269-10280). This method was not sufficient for unambiguous determination of charges of aliphatic hydrogens, values in the range of 0.03-0.09 gave almost similar fits. Since we found that the value of Q(H)=0.045 gives a good description of conformational energies of secondary alcohols, this value was fixed in RESP calculations during the determination of charges for the α-carbon, α-hydrogens, oxygen and hydroxyl hydrogen in alcohols.

Several van der Waals parameters for the 12-6 potential were modified based on Monte Carlo simulations of bulk liquids. Liquid simulations of cyclopentane, cyclohexane, n-pentane, isopentane, methanol, ethanol, 1-propanol, 2-propanol, sec-butanol, tert-butanol, oxane, isopropyl methyl ether, methyl tert-butyl ether, and diethyl ether were performed at room temperature in the periodic box similarly to that described previously (Jorgensen, W. L. et al., J Am Chem Soc 1996, 118:11225-11236). Isobutane, n-butane, neopentane, dimethyl ether, and ethyl methyl ether were simulated at their corresponding boiling temperatures. In our simulations, cubic boxes of at least 30 Å in length were employed which allowed use of nonbonded cutoffs of 14-15 Å. Each liquid simulation consisted of at least 4·10⁶ configurations of equilibration and 14-24·10⁶ configurations of averaging. Each simulation of an isolated molecule was done by equilibrating for 2·10⁶ conformations and averaging for at least 8·10⁶ conformations. All molecules were fully flexible during simulations. For each molecule, 10-25 simulations were performed by varying the nonbonded parameters and the optimal parameter set was determined by fitting the calculated densities and heats of vaporization to the corresponding experimental values.

The main parameterization set for torsional force constants included MP2/TZ//DZ rotational profiles for ethane, propane, isobutane, isopentane, neopentane, 2,2-dimethylbutane, methanol, ethanol, 1-propanol, 2-propanol, tert-butanol, dimethyl ether, ethyl methyl ether, methyl propyl ether, methoxymethanol, 3-hydroxy-2-oxopropanal, 3-methoxy-2-oxopropanal, methyl formate, methyl acetate, ethyl acetate, methyl propionate, and propyl formate. Also, the relative energies of all stable conformers of pentane, sec-butanol, cyclohexanol, diethyl ether, isopropyl methyl ether, methoxyethanol, and isopropyl formate were included along with the data on low-energy conformers of THPOH, HMOP, GTHP, and GTHPO. This set was supplemented with reliable experimental data or recently published ab initio energies for ethane (Hirota, E. et al., J Chem Phys 1979, 71, 1183-1187; Goodman, L. et al., Acc Chem Res 1999, 32, 983-993), butane (Allinger, N. L. et al., J Chem Phys 1997, 106, 5143-5150), cyclohexane (Dixon, D. A. and Komornicki, A. J Phys Chem 1990, 94, 5630-5636), cyclooctane (Rocha, W. R. et al., J Comput Chem 1998, 19, 524-534), methylcyclohexane (Wiberg, K. B. et al., J Org Chem 1999, 64, 2085-2095), cyclohexanol (Halgren, T. A. J Comput Chem 1999, 20, 730-748), dimethyl ether (Pophristic, V. and Goodman, L. J Phys Chem A 2000, 104, 3231-3238), ethyl methyl ether (Tsuzuki, S. et al., J Mol Struct (THEOCHEM) 1996, 366, 89-96), oxane (Freeman, F. et al., J Mol Struct (THEOCHEM) 2000, 496, 19-30; Smith, B. J. J Phys Chem A 1998, 102, 3756-3761), dimethoxyethane (Williams, D. J. and Hall, K. B. J Phys Chem 1996, 100, 8224-8229), methyl formate (Wiberg, K. B. et al., J Mol Struct 1999, 485-486, 239-248), methyl acetate (Sheridan, J. et al., A. J Mol Spectrosc 1980, 80, 1-11), and ethyl formate (Bohn, R. K. and Wiberg, K. B. Theor Chem Acc 1999, 102, 272-278). Finally, torsional profiles and conformational energies for diketones and ketoamides studied by us previously (Kahn, K. and Bruice, T. C. Bioorg Med Chem 2000, 8, 1881-1891) were included. The OPLS-AA torsional parameters were derived by iterative fitting of OPLS-AA calculated energy profiles for rotation around single bonds to the corresponding ab initio energy profiles. In the later stages, conformational analysis of all molecules in the parameterization set was done, and when discrepancies occurred, refitting to torsional profiles was performed.

Conformational Analysis of Polyketides.

The new parameters were used to perform conformational analysis of selected polyketides starting with a previously generated library of small rapamycin analogs (Adalsteinsson, H. and Bruice, T. C. Bioorg Med Chem 2000, 8: 625-635). Using this approach, tri- and tetraketides corresponding to analogs of FK506 (methoxy substituent at the position 31) were studied. The stable conformers were identified using the stochastic conformational searching facility in BOSS42. For each molecule, 15,000 trial structures were generated by moving every atom with the upper bound radius of 2 Å. Three criteria were used for retaining unique conformers. To satisfy the first criterion, the energy of a given conformer had to be more than 0.01 kcal/mol different from the energy of any previous conformer. Second, the conformer had to show a RMS deviation above 0.5 Å from any previous conformer when the two structures were superimposed. Last, the sum of squared internuclear distances between all possible atom pairs in the given conformer had to be more than 4.0 Å² different from the value found in any other conformer. Structures which satisfied all three criteria and were local minima on the potential energy surface were considered to be unique conformers. The low-energy ketide structures were compared to the crystal structure of bound FK506 (PDB code 1FKF). The lowest energy conformer of the tetraketide showing the highest similarity to the drug FK506 was docked to the average solution structure of the rabbit FKBP52 (also known as FKBP59, PDB code 1ROT) (Craescu, C. T. et al., J. Biochemistry 1996, 35, 11045-11052) using the program SYBYL (Tripos, Inc.) in order to confirm its ability to fit to the binding pocket.

Structures and Conformational Barriers in Model Compounds.

The MP2/aug-cc-pVTZ optimized structures of low energy conformers for some alkanes, alcohols, ethers, hemiacetals, and esters are shown in FIG. 20. The MP2/aug-cc-pVTZ optimized geometries are in very good agreement with experimental structures in cases where a comparison is available while the MP2/aug-cc-pVDZ method systematically overestimates the length of carbon-oxygen bonds.

Table 3 lists the ab initio conformational energies for compounds which were used for the determination of torsional force constants. For comparison, data from previously published research, and results of OPLS-AA calculations with newly optimized parameters are also given. When comparing the experimental and calculated data one should remember that ab initio torsional barriers correspond to the energy difference between the saddle point and the bottom of the potential well while the experimental data usually contains the contribution of zero point energy (ZPVE).

TABLE 3
Comparison of ab initio (MP2/aug-cc-pVDZ and MP2/TZ//DZ) and
OPLS-AA conformational energies and rotational barriers.
Energies (in kcal/mol) are given relative to the global minimum (GM).

| Compound (conf.)^{a} | DZ | TZ//DZ | Other data | OPLS-AA |
|---|---|---|---|---|
| Alkanes | | | | |
| Ethane (s_{TS}-a) | 3.02 | 2.90 | 2.89^{TZ}; 2.88-2.90^{exp}^{⁵⁰}; 2.79^{ai}^{⁵¹} | 2.84 |
| Propane (s_{TS}-a) | 3.23 | 3.20 | 3.19^{TZ}; 3.01^{TZ+ZPVE}; 3.26^{exp}^{⁶⁴} | 3.08 |
| Butane (g-a) | N/A | N/A | 0.62^{ai}^{⁴²}; 0.67 ± 0.10^{exp}^{⁶⁵} | 0.71 |
| Butane (e_{TS}-a) | N/A | N/A | 3.31^{ai}^{⁴²}; 3.62 ± 0.06^{exp}^{⁶⁵} | 3.30 |
| Butane (s_{TS}-a) | N/A | N/A | 5.50^{ai}^{⁴²} | 5.13 |
| Isobutane (s_{TS}-a) | 3.62 | 3.53 | 3.52^{TZ}; 3.90^{exp}^{⁶⁶} | 3.39 |
| Pentane (ag-aa) | 0.49 | 0.52 | 0.46^{exp}^{⁶⁷}; 0.76^{ai}^{⁶⁸} | 0.81 |
| Pentane (gg-aa) | 0.67 | 0.69 | 1.36^{ai}^{⁶⁸} | 1.48 |
| Isopentane (g-t) | 0.83 | 0.75 | 0.81^{exp}^{⁶⁹} | 0.54 |
| Isopentane (e_{TS}-t) | 3.01 | 3.07 | NA | 2.74 |
| Isopentane (s_{TS}-t) | 5.39 | 5.31 | NA | 4.68 |
| Neopentane (s_{TS}-a) | 4.12 | 3.90 | 4.29^{exp}^{⁷⁰} | 3.88 |
| 2,2-dimethylbutane (s_{TS}-a) | 5.12 | 5.02 | 4.50^{exp}^{⁷¹}; 5.20 ± 0.20^{exp}^{⁷²} | 4.63 |
| Cyclohexane (ch-tw.bt) | N/A | N/A | 6.90^{ai}^{⁵²} | 7.01 |
| Cyclooctane (crown-bt.ch) | N/A | N/A | 1.72^{ai}^{⁵³} | 0.79 |
| Cyclooctane (tw.bt.ch-bt.ch) | N/A | N/A | 1.73^{ai}^{⁵³} | 1.76 |
| Methylcyclohexane (ax-eq) | N/A | N/A | 1.76^{exp}^{⁵⁴} | 1.64 |
| Methylcyclohexane (CH₃:eq) | N/A | N/A | 3.01^{ai}^{⁵⁴} | 3.40 |
| Methylcyclohexane (CH₃:ax) | N/A | N/A | 2.42^{ai}^{⁵⁴} | 3.30 |
| Alcohols | | | | |
| Methanol (s_{TS}-a) | 1.15 | 1.00 | 1.065^{exp}^{^{73,74}} | 1.03 |
| Ethanol (g-a) | 0.26 | 0.22 | 0.13^{exp}^{⁷⁵}; 0.05^{ai}^{⁷⁶} | 0.29 |
| Ethanol (s_{TS}-a) | 1.53 | 1.32 | 1.27^{exp}^{⁷⁵}; 1.26^{ai}^{⁷⁶} | 1.48 |
| Ethanol (e_{TS}-a) | 1.30 | 1.15 | 1.15^{exp}^{⁷⁵}; 1.15^{ai}^{⁷⁶} | 1.07 |
| Ethanol (CH₃:a) | 3.39 | 3.30 | 3.39^{exp}^{⁷⁵}; 3.31^{ai}^{⁷⁶} | 3.20 |

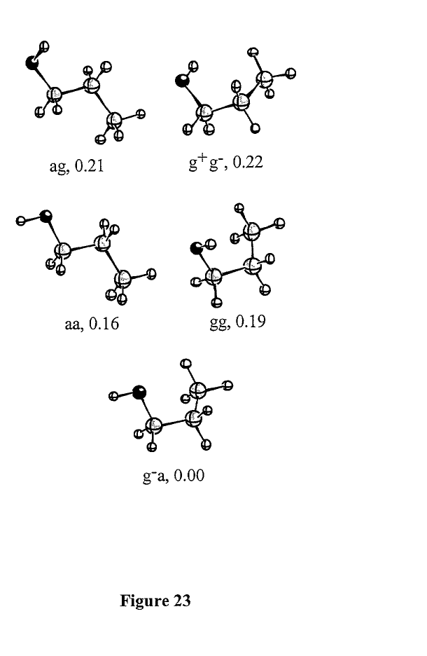

1-Propanol. GM is g⁻a with CC—CO −62.6° and CC—OH 179.5. FIG. 23.

| | | | | |
|---|---|---|---|---|
| 1-PrOH (aa-g⁻a) | 0.23 | 0.17 | 0.16^{TZ} | −0.34 |
| 1-PrOH (g⁻g⁻-g⁻a) | 0.26 | 0.20 | 0.19^{TZ} | −0.30 |
| 1-PrOH (ag-g⁻a) | 0.35 | 0.22 | 0.21^{TZ} | −0.13 |
| 1-PrOH (g⁻g⁺-g⁻a) | 0.34 | 0.23 | 0.22^{TZ} | 0.03 |
| 1-PrOH (sa_{TS}-g⁻a) | 5.31 | 5.11 | | 4.85 |
| 1-PrOH (ea_{TS}-g⁻a) | 3.61 | 3.35 | | 3.40 |
| 1-PrOH (g⁻e⁺_{TS}-g⁻a) | 1.08 | 0.82 | | 0.86 |
| 1-PrOH (g⁻e⁻_{TS}-g⁻a) | 1.16 | 0.96 | | 1.05 |
| 1-PrOH (sg⁻_{TS}-g⁻g⁻) | 5.18 | 4.98 | | 4.45 |
| 1-PrOH (e⁺g⁻_{TS}-g⁻g⁻) | 3.94 | 3.72 | | 3.33 |
| 1-PrOH (e⁻g⁻_{TS}-g⁻g⁻) | 3.75 | 3.52 | | 3.60 |
| 1-PrOH (as_{TS}-aa) | 1.37 | 1.08 | | 1.40 |
| 1-PrOH (ae_{TS}-aa) | 1.20 | 0.95 | | 1.03 |
| 1-PrOH (CH₃:aa) | 2.70 | 2.64 | 2.73 ± 0.06^{exp}^{⁷⁷} | 2.45 |

2-Propanol. GM is gauche (g) with HC—OH dihedral of ±62.6°

| | | | | |
|---|---|---|---|---|
| 2-PrOH (a-g) | 0.36 | 0.35 | 0.28^{exp}^{³²}; 0.45 ± 0.22^{exp}^{⁷⁸} | 0.28 |
| 2-PrOH (s_{TS}-g) | 1.39 | 1.33 | | 0.80 |
| 2-PrOH (e_{TS}-g) | 1.36 | 1.18 | | 1.21 |
| 2-PrOH (CH₃:g) | 3.09 | 3.30 | | 3.44 |
| tert-Butanol (s_{TS}-a) | 1.34 | 1.21 | 1.27^{exp}^{⁷⁹} | 0.96 |
| tert-Butanol (CH₃:a) | 3.55 | 3.46 | | 3.77 |

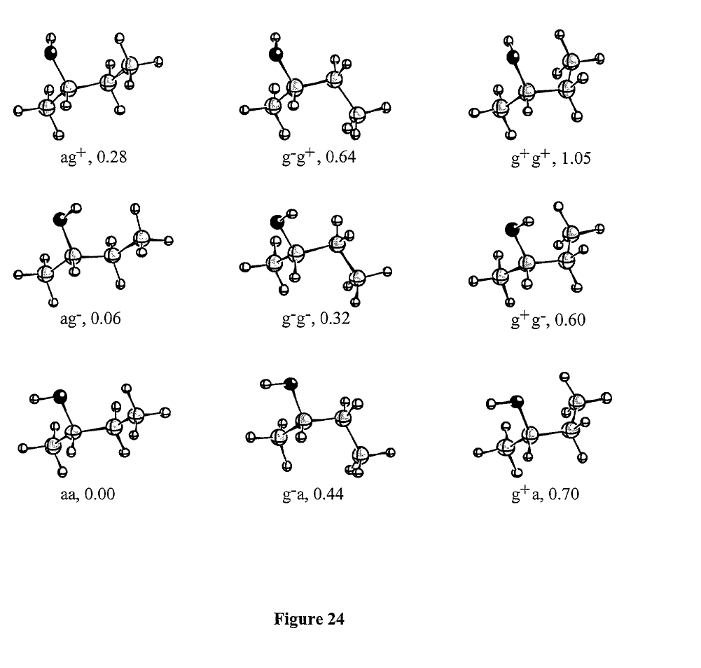

(S)-2-Butanol. GM is anti-anti (aa) with CC—CC 180.0 and C_{CH2} C—OH 180.0. FIG. 24.

| | | | | |
|---|---|---|---|---|
| 2-ButOH (ag⁻-aa) | 0.12 | 0.06 | −0.14^{ai}^{^{80,81}} | −0.60 |
| 2-ButOH (ag⁺-aa) | 0.36 | 0.28 | −0.18^{ai}^{^{80,81}} | 0.03 |
| 2-ButOH (g⁻g⁻-aa) | 0.40 | 0.32 | 0.65^{ai}^{^{80,81}} | 0.28 |
| 2-ButOH (g⁻a-aa) | 0.50 | 0.44 | 0.73^{ai}^{^{80,81}} | 0.36 |
| 2-ButOH (g⁺g⁻-aa) | 0.71 | 0.60 | 0.97^{ai}^{^{80,81}} | 0.52 |
| 2-ButOH (g⁻g⁺-aa) | 0.79 | 0.64 | 0.61^{ai}^{^{80,81}} | 0.55 |
| 2-ButOH (g⁺a-aa) | 0.77 | 0.70 | 1.13^{ai}^{^{80,81}} | 0.80 |
| 2-ButOH (g⁺g⁺-aa) | 1.27 | 1.05 | 1.24^{ai}^{^{80,81}} | 0.48 |
| cyclohexanol (eq/CS-eq/C1) | 0.21 | 0.26 | 0.18^{ai}^{³²} | 0.14 |
| cyclohexanol (ax/C1-eq/C1) | 0.36 | 0.45 | 0.33^{ai}^{³²}; 0.58^{exp}^{³²} | 1.07 |
| cyclohexanol (ax/CS-eq/gC1) | 1.20 | 1.20 | 1.14^{ai}^{³²} | 0.21 |
| Ethers | | | | |
| Dimethyl ether (s_{TS}-a) | 2.60 | 2.62 | 2.69^{exp}^{⁸²}; 2.60^{exp}^{⁸³} | 2.52 |

Ethyl methyl ether. GM is anti with CC—OC 180.0°.

| | | | | |
|---|---|---|---|---|
| EME (g-a) | 1.29 | 1.37 | 1.23^{exp}^{⁸⁴}; 1.35^{ai}^{⁵⁶} | 1.26 |
| EME (s_{TS}-a) | 6.25 | 6.39 | 7.05^{ai}^{⁵⁶} | 6.69 |
| EME (e_{TS}-a) | N/A | N/A | 2.58^{ai}^{⁵⁶} | 2.42 |
| EME (C—CH₃:a) | 3.16 | 3.08 | 3.08^{exp}^{⁸⁵}; 3.14^{exp}^{⁸⁶} | 3.04 |
| EME (O—CH₃:a) | 2.51 | 2.44 | 2.61^{exp}^{⁸⁵}; 2.46^{exp}^{⁸⁶} | 2.52 |

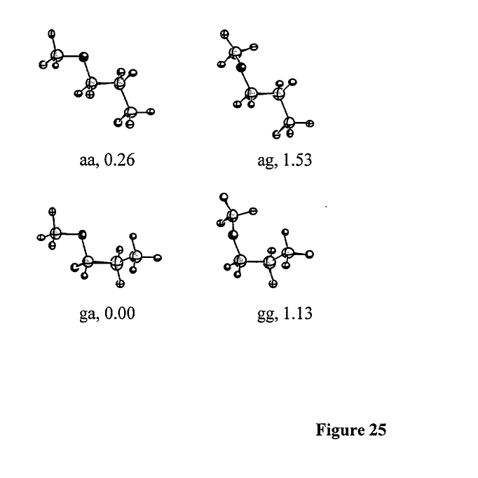

Methyl propyl ether. GM is ga with CC—CO at 62.4° and CC—OC −179.1°. FIG. 25.

| | | | | |
|---|---|---|---|---|
| MPE (aa-ga) | 0.32 | 0.26 | | 0.02 |
| MPE (gg-ga) | 1.00 | 1.13 | | 1.13 |
| MPE (ag-ga) | 1.45 | 1.53 | | 1.31 |
| MPE (sa_{TS}-ga) | 5.01 | 5.04 | | 4.37 |
| MPE (ea_{TS}-ga) | 3.44 | 3.41 | | 3.05 |
| MPE (as_{TS}-ga) | 6.37 | 6.52 | | 6.86 |
| DEE (ag-aa) | 1.22 | 1.36 | 1.15^{exp}^{⁶⁷} | 1.31 |
| DEE (gg-aa) | 2.49 | 2.78 | | 2.60 |

Isopropyl methyl ether. GM has C1 symmetry with CC—OC 68.4° and −169.4°.

| | | | | |
|---|---|---|---|---|
| IME (g-a) | 1.92 | 2.54 | | 1.81 |
| IME (O—CH₃:a) | 1.80 | N/A | 1.73^{exp}^{⁸⁷} | 2.11 |
| oxane (tw.bt-chair) | 5.57 | 5.65 | 5.64^{ai}^{⁵⁸} | 7.07 |
| dimethoxyethane (asa_{TS}-aaa) | N/A | N/A | 9.51, 8.90^{ai}^{⁵⁹} | 9.11 |
| dimethoxyethane (aga-aaa) | N/A | N/A | 0.51, 0.15^{ai}^{⁵⁹} | 0.38 |
| Hemiacetals | | | | |

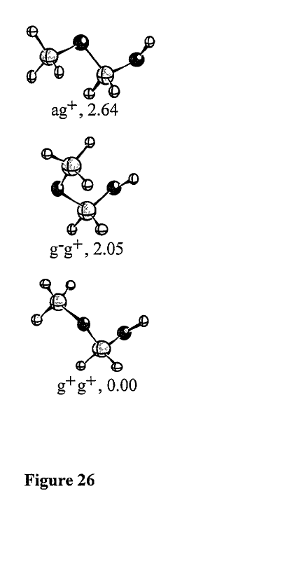

Methoxymethanol. GM is g⁺g⁺ with CO—CO at 67.5° and OC—OH at 64.7. FIG. 26.

| | | | | |
|---|---|---|---|---|
| MMeOH (g⁻g⁺-g⁺g⁺) | 2.09 | 2.09 | 2.05^{TZ}, 2.8^{ai}^{⁸⁸} | 2.15 |
| MMeOH (ag⁺-g⁺g⁺) | 2.73 | 2.62 | 2.64^{TZ}, 3.3^{ai}^{⁸⁸} | 2.58 |
| MMeOH (sg⁺_{TS}-g⁺g⁺) | 7.14 | 7.09 | | 7.24 |
| MMeOH (aa_{TS}-g⁺g⁺) | 6.54 | 6.43 | 6.42^{TZ}, 8.2^{ai}^{⁸⁸} | 6.52 |
| MMeOH (O—CH₃:g⁺g⁺) | 1.65 | 1.51 | | 1.88 |
| MMeOH (O—CH₃:aa) | 2.31 | 2.30 | | 1.97 |

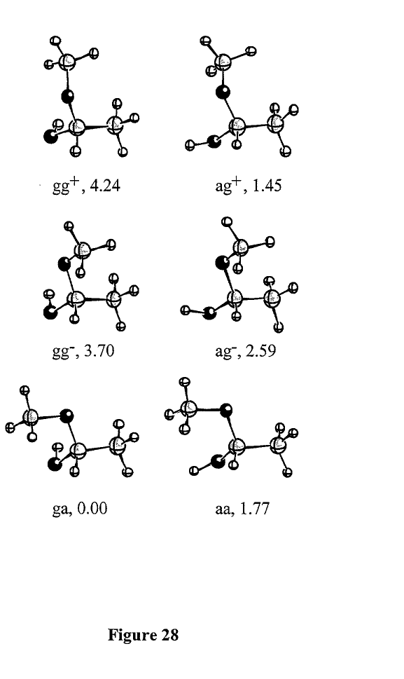

(R)-1-Methoxyethanol. GM is g⁻t with CCOH −55°, and CC—OC −171°. FIG. 28.

| | | | |
|---|---|---|---|
| MEtOH (tg⁺-g⁻t) | 1.27 | 1.45 | 1.46 |
| MEtOH (tt-g⁻t) | 1.79 | 1.77 | 1.85 |
| MEtOH (tg⁻-g⁻t) | 2.62 | 2.59 | 3.59 |
| MEtOH (gg⁻-g⁻t) | 3.80 | 3.70 | 3.50 |
| MEtOH (gg⁺-g⁻t) | 4.21 | 4.24 | 3.79 |
| MEtOH (C—CH₃:g⁻t) | 3.22 | 3.22 | 2.99 |
| MEtOH (C—CH₃:tt) | 2.80 | 2.80 | 3.00 |

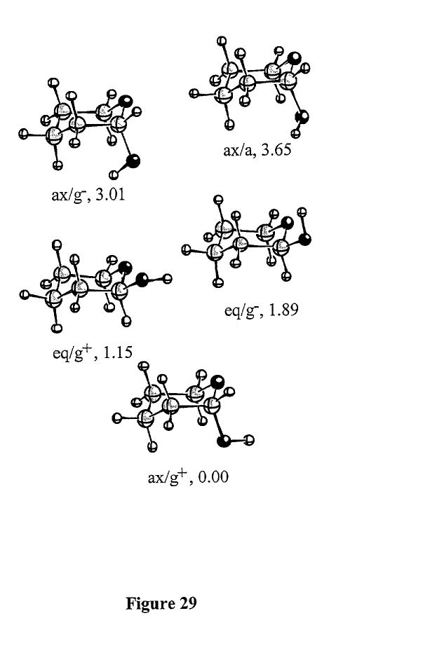

(S)-Tetrahydropyran-2-ol. GM is axial gauche (ax/g⁺) with CO—CO 64.4° and
OC—OH 56.7°. FIG. 29.

| | | | | |
|---|---|---|---|---|
| THPOH (eq/g⁺-ax/g⁺) | 1.15 | N/A | 1.3^{ai}^{⁸⁸} | 1.43 |
| THPOH (eq/g⁻-ax/g⁺) | 1.89 | N/A | 2.2^{ai}^{⁸⁸} | 1.55 |
| THPOH (ax/g⁻-ax/g⁺) | 3.01 | N/A | 4.0^{ai}^{⁸⁸} | 1.62 |
| THPOH (ax/a-ax/g⁺) | 3.65 | N/A | 4.4^{ai}^{⁸⁸} | 3.55^{TS} |
| THPOH (aa_{TS}-ax/g⁺) | 5.28 | N/A | | 5.57^{TS} |
| Esters. | | | | |
| Methyl formate (E-Z) | 5.48 | 5.45 | 5.45^{TZ}; 4.75 ± 0.19^{exp}^{⁸⁹}; 5.65^{ai}^{³²} | 4.50 |
| Methyl formate (O—CH₃:Z) | 1.25 | 1.12 | 1.11^{TZ}; 1.21^{exp} 1.19^{exp}^{⁶⁰} | 1.21 |
| Methyl acetate (E-Z) | 7.33 | 7.47 | 8.5 ± 1.0^{exp}^{⁸⁹} | 8.11 |
| Methyl acetate (C—CH₃:Z) | 0.20 | 0.19 | 0.29^{exp}^{⁶¹} | 0.27 |
| Methyl acetate (O—CH₃:Z) | 1.37 | 1.17 | 1.22^{exp}^{⁶¹} | 1.24 |
| Methyl propionate (E-Z) | N/A | N/A | 8.75^{ai}^{⁹⁰} | 8.33 |
| Methyl propionate (O—CH₃:Z) | 1.36 | 1.17 | | 1.26 |
| Methyl propionate (C—CH₃:Z) | 2.51 | 2.47 | | 2.33 |
| Ethyl formate (Ea-Za) | 5.10 | 5.06 | 3.2^{exp}^{⁹¹} | 4.00 |
| Ethyl formate (Zg-Za) | N/A | N/A | 0.19 ± 0.6^{exp}^{⁹²}; 0.34^{ai}^{³²} | 0.24 |
| Ethyl acetate (Zg-Za) | N/A | N/A | 0.32^{exp}^{⁹³} | 0.25 |
| Isopropyl formate (Zg-Za) | 2.35 | 2.42 | 2.43^{ai}^{⁹⁴} | 2.96 |
| Isopropyl formate (Ea-Za) | 4.71 | 4.61 | | 3.64 |
| Isopropyl formate (Eg-Za) | 6.99 | 7.00 | | 5.83 |
| Propyl formate (Zga-Zag) | 0.03 | 0.09 | | 0.23 |
| Propyl formate (Zg⁺g⁺-Zag) | −0.01 | 0.14 | | 0.05 |
| Propyl formate (Zaa-Zag) | 0.25 | 0.22 | | 0.08 |
| Glyoxal derivatives | | | | |

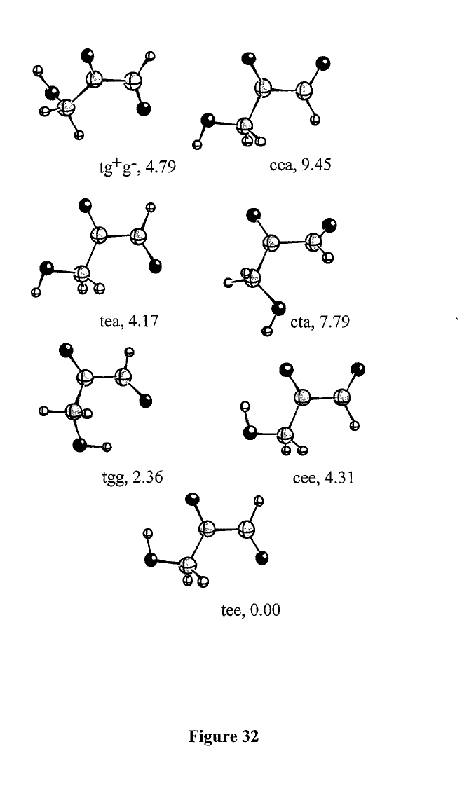

3-Hydroxy-2-oxopropanal. FIG. 32.

| | | | |
|---|---|---|---|
| HOP (tgg-tee) | 1.97 | 2.36 | 2.52 |
| HOP (tea-tee) | 3.83 | 4.17 | 4.14 |
| HOP (tg⁺g⁻-tee) | 4.24 | 4.79 | 4.31 |
| HOP (cee-tee) | 4.52 | 4.31 | 5.12 |
| HOP (cta-tee) | 7.42 | 7.79 | 6.54 |
| HOP (cea-tee) | 9.30 | 9.45 | 9.86 |

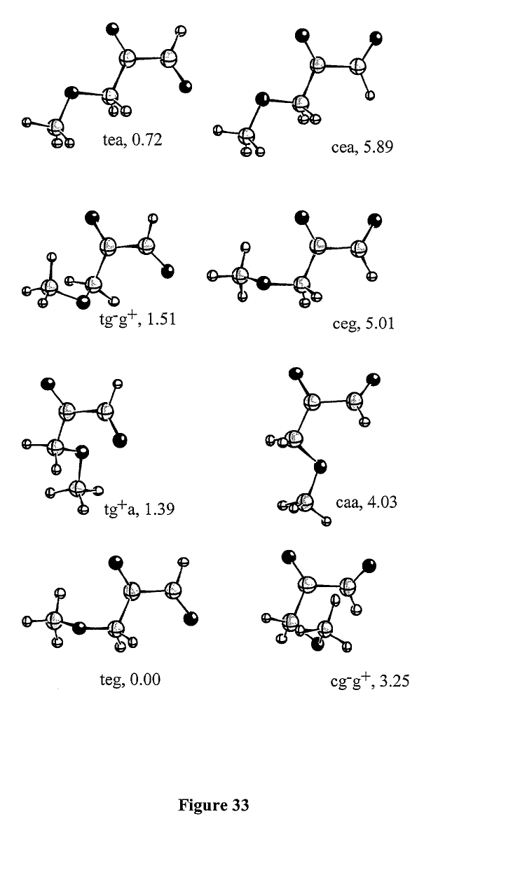

3-Methoxy-2-oxopropanal. FIG. 33.

| | | | |
|---|---|---|---|
| MOP (tea-teg) | 0.92 | 0.72 | 0.72 |
| MOP (tg⁻g⁺-teg) | 1.18 | 1.51 | 1.11 |
| MOP (tg⁺a-teg) | 1.33 | 1.39 | 1.18 |
| MOP (cg⁻g⁺-teg) | 3.28 | 3.25 | 1.38 |
| MOP (caa-teg) | 4.18 | 4.03 | 3.40 |
| MOP (ceg-teg) | 5.17 | 5.01 | 4.96 |
| MOP (cea-teg) | 6.29 | 5.89 | 6.19 |

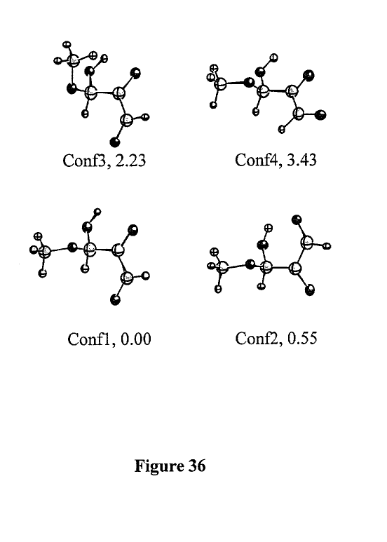

3-Hydroxy-3-methoxy-2-oxopropanal. FIG. 36.

| | | | |
|---|---|---|---|
| HMOP (Conf2-Conf1) | 0.47 | 0.55 | 1.76 |
| HMOP (Conf3-Conf1) | 1.98 | 2.23 | 2.33 |
| HMOP (Conf4-Conf1) | 3.56 | 3.43 | 2.32 |
| HMOP (O—CH₃:Conf1) | 1.82 | 1.75 | 1.83 |

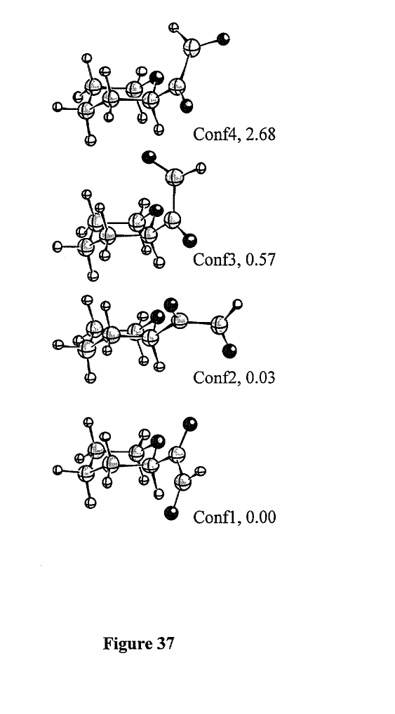

2-glyoxoyl-tetrahydropyran. FIG. 37.

| | | | |
|---|---|---|---|
| GTHP (Conf2-Conf1) | 0.03^{G} | N/A | −0.23 |
| GTHP (Conf3-Conf1) | 0.57^{G} | N/A | 1.10 |
| GTHP (Conf4-Conf1) | 2.68^{G} | N/A | 2.31 |

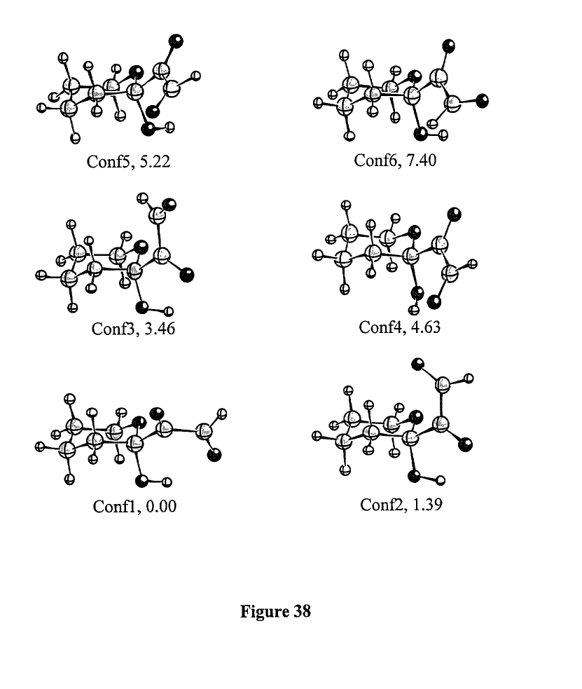

2-glyoxoyl-tetrahydropyran-2-ol. FIG. 38.

| GTHPO (Conf2-Conf1) | 1.38^{G} | N/A | 0.78 |
|---|---|---|---|
| GTHPO (Conf3-Conf1) | 3.46^{G} | N/A | −0.12 |
| GTHPO (Conf4-Conf1) | 4.63^{G} | N/A | 3.20 |
| GTHPO (Conf5-Conf1) | 5.22^{G} | N/A | 3.38 |
| GTHPO (Conf6-Conf1) | 7.40^{G} | N/A | 3.78 |
| ^{a}Following symbols, based on a recent recommendation (Michl, J.; West, R. Acc Chem Res 2000, 33, 821-823), are used to define the conformers and torsional barriers: a—anti (θ = 180°); e—eclipsing (θ ±120°); g—gauche (θ ±60°); s—syn (θ= 0°); t—transoid (θ ±165°); CH₃: a—barrier for the rotation of a terminal methyl group in the anti conformer. Conformation of the cyclic structures are designated as: | | | |
| # ch—chair; tw—twist; bt—boat; eq—substituent occupies an equatorial position; ax—substituent occupies an axial position. | | | |
| # The symbols C1 and CS indicate molecular symmetry. The rotation around the O═C(sp²)—O(sp³)—C(sp³) dihedral in esters is described using symbols Z (cis, 0°) and E (trans, 180°). The bond involved in the methyl group rotation in esters is explicity shown, e.g. O—CH₃:Z designates a rotation of the methoxy group in the Z conformer. | | | |
| # A subscript TS in the first column indicates that the structure corresponds to the top of the rotational barrier, the superscript TZ indicates that both energy evaluation and optimization were performed at the MP2/aug-cc-pVTZ level, and | | | |
| # subscript G designates optimization with the 6-31 + G(d,p) basis. | | | |

(a) Following symbols, based on a recent recommendation (Michl, J.; West, R. Acc Chem Res 2000, 33, 821-823), are used to define the conformers and torsional barriers: a—anti (θ=180°); e—eclipsing (θ±120°); g—gauche (θ±160°); s—syn (θ=0°); t—transoid (θ±165°); CH₃:a—barrier for the rotation of a terminal methyl group in the anti conformer. Conformations of cyclic structures are designated as: ch—chair; tw—twist; bt—boat; eq—substituent occupies an equatorial position; ax—substituent occupies an axial position. The symbols Cl and CS indicate molecular symmetry. The rotation around the O═C(sp²)—O(sp³)—C(sp³) dihedral in esters is described using symbols Z (cis, 0°) and E (trans, 180°). The bond involved in the methyl group rotation in esters is explicitly shown, e.g. O—CH₃:Z designates a rotation of the methoxy group in the Z conformer. A subscript TS in the first column indicates that the structure corresponds to the top of the rotational barrier, the superscript TZ indicates that both energy evaluation and optimization were performed at the MP2/aug-cc-pVTZ level, and subscript G designates optimization with the 6-31+G(d,p) basis.

### REFERENCES FOR TABLE 3

32) Halgren, T. A. J Comput Chem 1999, 20, 730-748.

42) Allinger, N. L.; Fermann, J. T.; Allen, W. D.; Schaefer, H. F. J Chem Phys 1997, 106, 5143-5150.

50) Hirota, E.; Saito, S.; Endo, Y. J Chem Phys 1979, 71, 1183-1187.

51) Goodman, L.; Pophristic, V.; Weinhold, F. Acc Chem Res 1999, 32, 983-993.

52) Dixon, D. A.; Komornicki, A. J Phys Chem 1990, 94, 5630-5636.

53) Rocha, W. R.; Pliego, J. R.; Resende, S. M.; Dos Santos, H. F.; De Oliveira, M. A.; De Almeida, W. B. J Comput Chem 1998, 19, 524-534.

54) Wiberg, K. B.; Hammer, J. D.; Castejon, H.; Bailey, W. F.; DeLeon, E. L.; Jarret, R. M. J Org Chem 1999, 64, 2085-2095.

55) Pophristic, V.; Goodman, L. J Phys Chem A 2000, 104, 3231-3238.

56) Tsuzuki, S.; Uchimaru, T.; Tanabe, K. J Mol Struct (THEOCHEM) 1996, 366, 89-96.

57) Freeman, F.; Kasner, J. A.; Kasner, M. L.; Hehre, W. J. J Mol Struct (THEOCHEM) 2000, 496, 19-30.

58) Smith, B. J. J Phys Chem A 1998, 102, 3756-3761.

59) Williams, D. J.; Hall, K. B. J Phys Chem 1996, 100, 8224-8229.

60) Wiberg, K. B.; Bohn, R. K.; Jimenez-Vazquez, H. J Mol Struct 1999, 485-486, 239-248.

61) Sheridan, J.; Bossert, W.; Bauder, A. J Mol Spectrosc 1980, 80, 1-11.

62) Bohn, R. K.; Wiberg, K. B. Theor Chem Acc 1999, 102, 272-278.

63) Kahn, K.; Bruice, T. C. Bioorg Med Chem 2000, 8, 1881-1891.

64) Hoyland, J. R. J Chem Phys 1968, 49, 1908-1912.

65) Herrebout, W. A.; van der Veken, B. J.; Wang, A.; Durig, J. R. J Phys Chem 1995, 99, 578-585.

66) Lide, D. R. J.; Mann, D. E. J Chem Phys 1958, 29, 914-920.

67) Kanesaka, I.; Snyder, R. G.; Strauss, H. L. J Chem Phys 1986, 84, 395-397.

68) Wiberg, K. B.; Murcko, M. A. J Am Chem Soc 1988,110, 8029-8038.

69) Verma, A. L.; Murphy, W. F.; Bernstein, H. J. J Chem Phys 1974, 60, 1540-1544.

70) Weiss, S.; Leroi, G. E. Spectrochim Acta A 1969, 25, 1759-1766.

71) Snyder, R. G.; Schachtschneider, J. H. Spectrochim Acta 1965, 21, 169-195.

72) Whalon, M. R.; Bushweller, C. H.; Anderson, W. G. J Org Chem 1984, 49, 1185-1190.

73) De Lucia, F. C.; Herbst, E.; Anderson, T.; Helminger, P. J Mol Spectrosc 1989, 134, 395-411.

74) Xu, L. H.; Lees, R. M.; Hougen, J. T. J Chem Phys 1999, 110, 3835-3841.

75) Durig, J. R.; Larsen, R. A. J Mol Struct 1989, 238, 195-222.

76) Senent, M. L.; Smeyers, Y. G.; Dominguez-Gómez, R.; Villa, M. J Chem Phys 2000, 112, 5809-5819.

77) Dreizler, H.; Scappini, F. Z Naturforsch 1981, 36A, 1187-1191.

78) Hirota, E. J Phys Chem 1979, 83, 1457-1465.

79) Suwa, A.; Ohta, H.; J Mol Struct 1988, 172, 275-290.

80) Xu, S.; Liu, Y.; Sha, G.; Zhang, C.; Xie, J. J Phys Chem A 2000, 104, 8671-8676.

81) Wang, F.; Polavarapu, P. L. J Phys Chem A 2000,104, 10683-10687.

82) Groner, P.; Durig, J. R. J Chem Phys 1977, 66, 1856-1874.

83) Durig, J. R.; Li, Y. S.; Groner, P. J Mol Spectrosc 1976, 62, 159-174.

84) Oyanagi, K.; Kuchitsu, K. Bull Chem Soc Jpn 1978, 51, 2237-2242.

85) Durig, J. R.; Compton, D. A. C. J Chem Phys 1978, 69, 4713-4719.

86) Perchard, J. P. J Mol Struct 1970, 6, 457-470.

87) Nakagawa, J.; Imachi, M.; Hayashi, M. J Mol Struct 1984, 112, 201-206.

88) Ganguly, B.; Fuchs, B. J Org Chem 1997, 62, 8892-8901.

89) Blom, C. E.; Günthard, H. H. Chem Phys Lett 1981, 84, 267-271.

90) Blomqvist, J.; Ahjopalo, L.; Mannfors, B.; Pietilä, L. -O. J Mol Struct (THEOCHEM) 1999, 488, 247-262.

91) Tanaka, Y. Acustica 1970, 23, 328-333.

92) Riveros, J. M.; Wilson, E. B., Jr. J Chem Phys 1967, 46, 4605-4612.

93) George, W. O.; Hassid, D. V.; Maddams, W. F. J Chem Soc, Perkin Trans 2 1972, 1798-1801.

94) Halgren, T. A.; Nachbar, R. B. J Comput Chem 1996, 17, 587-615.

Force Field Parameters.

Minimization of molecules shown in FIG. 20, using standard OPLS-AA parameters revealed that the bond stretching parameters can be improved by introducing individual bond length parameters for the carbon-oxygen single bond in alcohols, ethers, and esters. The standard parameter set assigned a common distance, 1.41 Å to all such bonds; this is too short for alcohols and esters. Optimal C—O distances were 1.423 Å for alcohols, 1.409 Å for ethers, and 1.437 Å for esters. The optimal lengths for the CT-OH and CT-OS bonds in hemiacetals were 1.404 Å, and 1.390 Å respectively. Standard bond bending parameters were found to be appropriate in most cases, and only some small adjustments of equilibrium angles were made. The CT-OH-HO angle was reduced from 108.5° to 108.2° and the CT-CT-OH angle was changed from 109.5 to 108.5° in order to improve agreement for alcohols. In addition, the CT-OS-CT angle in ethers was reduced from 109.5° to 108.6°. Values of 106.2° and 109.5° were assigned for CG-CT-OS and CG-CT-OH angles, respectively, to describe glyoxal derivatives.

Reproduction of ab initio conformational energies of some cyclic molecules, such as cyclooctane, cyclohexanol, tetrahydropyran-2-ol, 2-glyoxoyl-tetrahydropyran, and 2-glyoxyl-tetrahydropyran-2-ol was not successful with the standard OPLS-AA parameters. For cyclohexanol, standard OPLS-AA predicts the axial conformer with CS symmetry to be the most stable, but ab initio calculations show that this conformer has the highest energy. The discrepancy in relative energies of the axial Cₛ and equatorial C₁ conformers was 2.03 kcal/mol. Cyclohexanol is a challenging molecule for force fields that use appreciable positive charges on aliphatic hydrogens (Halgren, T. A., J Comput Chem 1999, 20: 730-748). After adjustments of torsional force constants failed to improve the agreement we investigated if the reduction of Q(H) from its standard value of 0.06, or changing the scaling factor for 1-4 electrostatic interactions, would improve the calculated conformational energies. It was found that the reduction of the hydrogen charge to 0.045 improved the agreement between OPLS-AA and ab initio results for secondary alcohols as well as for cyclic alkanes. Change of the charge of the aliphatic hydrogen, which is one of the basic parameters of the OPLS-AA force field, necessitated extensive re-determination of other force field parameters. Based on RESP calculations we adjusted charges of the hydroxyl oxygen and the hydroxyl hydrogen to −0.66 and +0.40, respectively. In addition, the charge on hydrogens adjacent to an sp³ oxygen was decreased further to 0.03. In the standard OPLS-AA force field such hydrogens had either charge of 0.10 (hemiacetals), 0.06 (most alcohols), 0.04 (methanol), or 0.03 (ethers, esters) (Jorgensen, W. L. et al., J Am Chem Soc 1996, 118: 11225-11236; Price, M. L. et al., J Comput Chem 2001, 22, 1340-1352). Bulk liquid simulations of primary alcohols, ethers, unbranched alkanes and cycloalkanes indicated that only minor adjustments of σ_{C} and ε_{C} values are necessary. Excellent fit to experimental bulk liquid data was obtained after increasing σ_{C} from 3.50 to 3.52 and raising ε_{C} from 0.066 to 0.067. However, agreement for branched molecules was still not satisfactory. After noting that larger errors for branched molecules occurred with the original OPLS-AA force field as well (Jorgensen, W. L. et al., J Am Chem Soc 1996, 118: 11225-11236), we decided to introduce unique nonbonded parameters for secondary and tertiary sp³ carbons. Concurrent optimization of nonbonded parameters and torsional force constants yielded final values shown in Tables 4 and 5. The predicted relative conformational energies are listed in the last column of Table 3 and bulk liquid densities and heats of vaporization are given in Table 6.

TABLE 4
Suggested nonbonded parameters for the OPLS-AA force field for the
description of alkanes, alcohols, ethers, hemiacetals, and esters.

| TYPE ATOM | Q | σ | ε |
|---|---|---|---|
| Alkanes | | | |
| CT CH₃ in alkane | −0.135 | 3.52 | 0.067 |
| CT CH₂ in alkane | −0.090 | 3.52 | 0.067 |
| CT CH in alkane | −0.045 | 3.38 | 0.059 |
| CT C in alkane | 0.000^{a} | 3.20 | 0.051 |
| HC H in alkanes | 0.045 | 2.50^{a} | 0.030^{a} |
| Alcohols | | | |
| CT CH₃ next to OH in methanol | 0.170 | 3.52 | 0.067 |
| CT CH₂ next to OH in primary alcohols | 0.200 | 3.52 | 0.067 |
| CT CH next to OH in secondary alcohols | 0.230 | 3.38 | 0.059 |
| CT C next to OH in tertiary alcohols | 0.260 | 3.20 | 0.051 |
| OH O hydroxyl in mono alcohols | −0.660 | 3.08 | 0.170^{a} |
| HO H hydroxyl in mono alcohols | 0.400 | 0.00^{a} | 0.000^{a} |
| HC H alpha to hydroxyl group | 0.030 | 2.50^{a} | 0.030^{a} |
| Ethers | | | |
| CT CH₃ next to OS in ethers | 0.110^{a} | 3.52 | 0.067 |
| CT CH₂ next to OS in ethers | 0.140^{a} | 3.52 | 0.067 |
| CT CH next to OS in secondary ethers | 0.170^{a} | 3.38 | 0.062 |
| CT C next to OS in tertiary ethers | 0.200^{a} | 3.20 | 0.051 |
| OS Ether oxygen | −0.400^{a} | 2.90^{a} | 0.140^{a} |
| HC H alpha to ether oxygen | 0.030^{a} | 2.50^{a} | 0.030^{a} |
| Hemiacetals | | | |
| CT CH₂O₂ in hemiacetal | 0.400 | 3.52 | 0.067 |
| CT CHRO₂ in hemiacetal | 0.430 | 3.38 | 0.059 |
| CT CR₂O₂ in hemiacetal | 0.460 | 3.20 | 0.051 |
| OH O hydroxyl in hemiacetals | −0.660 | 3.08 | 0.170^{a} |
| HO H hydroxyl in hemiacetals | 0.400 | 0.00^{a} | 0.000^{a} |
| OS Ether oxygen in hemiacetals | −0.400^{a} | 2.90^{a} | 0.140^{a} |
| HC H alpha to oxygens | 0.030 | 2.50^{a} | 0.030^{a} |
| Esters (for the alkoxy moiety) | | | |
| CT CH₃ in methyl esters | 0.160 | 3.52 | 0.067 |
| CT CH₂ in ethyl esters | 0.190 | 3.52 | 0.067 |
| CT CH in isopropyl esters | 0.220 | 3.38 | 0.059 |
| CT C in tert-butyl esters | 0.250 | 3.20 | 0.051 |
| OE Alkoxy oxygen in esters | −0.330^{a} | 3.00^{a} | 0.170^{a} |
| HC H alpha to oxygen | 0.030^{a} | 2.42^{a} | 0.015^{a} |
| Glyoxal derivatives | | | |
| CG Carbonyl C adjacent to CT/CO | 0.360^{b} | 3.75^{a} | 0.105^{a} |
| CG Carbonyl C adjacent to HC | 0.430^{b} | 3.75^{a} | 0.105^{a} |
| OG Carbonyl O adjacent to CT/CO | −0.360^{b} | 2.96^{a} | 0.210^{a} |
| OG Carbonyl O adjacent to HC | −0.430^{b} | 2.96^{a} | 0.210^{a} |
| HC Hydrogen connected to GG | 0.000 | 2.42^{a} | 0.015^{a} |

^{a}Value is unchanged from the standard OPLS-AA force field value.
^{b}Values are changed from our previously recommended value ±0.40 (Kahn, K. and Bruice, T. C. Bioorg Med Chem 2000, 8, 1881-1891) based on RESP calculations and preliminary liquid simulations of glyoxal and dimethylglyoxal.

TABLE 5
Suggested torsional parameters for alkanes, alcohols, ethers, hemiacetals, and
esters to be used with new nonbonded parameters based on Q(H) = 0.045.

| TYPE | V1 | V2 | V3 | V4 | Parameterization data |
|---|---|---|---|---|---|
| Alkanes | | | | | |
| NC—CT—CT—NC | 0.00 | 0.00 | 0.30 | 0.00 | ethane |
| HC—CT—CT—CT | 0.00 | 0.00 | 0.32 | 0.00 | propane, isobutane, neopentane |
| CT—CT—CT—CT | 0.75 | 0.20 | 0.25 | −0.25 | butane, pentane, isopentane, cyclohexane, cyclooctane, |
| Alcohols | | | | | |
| HC—CT—OH—HO | 0.00 | 0.00 | 0.34 | 0.00 | MeOH, EtOH, iPrOH, cHexOH |
| CT—CT—OH—HO | −0.20 | 0.04 | 0.32 | −0.06 | EtOH, 1-PrOH, iPrOH, tBuOH, cHexOH |
| HC—CT—CT—OH | 0.00 | 0.00 | 0.34 | 0.00 | EtOH, iPrOH, tBuOH |
| CT—CT—CT—OH | 1.35 | −0.08 | 0.29 | −0.04 | 1-PrOH, 2-BuOH, cHexOH |
| Ethers | | | | | |
| HC—CT—OS—CT | 0.00 | 0.00 | 0.67 | 0.00 | dimethyl ether, EME, IME |
| CT—CT—OS—CT | −0.10 | −0.10 | 0.70 | −0.05 | EME, DEE, IME, oxane |
| HC—CT—CT—OS | 0.00 | 0.00 | 0.31 | 0.00 | EME, DEE, MPE |
| CT—CT—CT—OS | 0.95 | −0.20 | 0.30 | −0.25 | MPE, oxane |
| Hemiacetals | | | | | |
| OS—CO—OH—HO | −0.40 | −2.15 | 0.40 | −0.35 | MMeOH, MEtOH |
| HC—CO—OH—HO | 0.00 | 0.00 | 0.34 | 0.00 | as in alcohols, MMeOH |
| CT—CO—OH—HO | −0.20 | 0.04 | 0.32 | −0.06 | as in alcohols, MEtOH |
| CT—OS—CO—OH | −1.30 | −1.80 | 1.10 | 0.00 | MMeOH, MEtOH |
| HC—CT—CO—OH | 0.00 | 0.00 | 0.40 | 0.00 | MEtOH |
| CT—CT—CO—OH | 1.35 | −0.08 | 0.29 | −0.04 | as in alcohols, THPOH |
| HC—CT—CO—OS | 0.00 | 0.00 | 0.44 | 0.00 | MEtOH |
| CT—CT—CO—OS | 0.95 | −0.20 | 0.30 | −0.25 | as in ethers, THPOH |
| HC—CT—OS—CO | 0.00 | 0.00 | 0.50 | 0.00 | MMeOH |
| CT—CT—OS—CO | −0.10 | −0.10 | 0.70 | −0.05 | as in ethers, THPOH |
| Esters | | | | | |
| HC—CT—CT—C | 0.00 | 0.00 | −0.11 | 0.00 | methyl propionate |
| CT—CT—C═O | −0.31 | 0.94 | −0.35 | 0.12 | methyl propionate |
| CT—CT—C—OE | 0.00 | 0.00 | −0.533 | 0.00 | standard OPLS-AA |
| HC—CT—C═O | 0.00 | 0.00 | 0.00 | 0.00 | methyl acetate |
| HC—CT—C—OE | 0.00 | 0.00 | 0.05 | 0.00 | methyl acetate |
| CT—C—OE—CT | 3.00 | 5.12 | 0.00 | 0.00 | methyl formate, methyl acetate, ethyl formate |
| C—OE—CT—HC | 0.00 | 0.00 | 0.08 | 0.00 | methyl formate, methyl acetate, methyl propionate |
| C—OE—CT—CT | −1.60 | −0.40 | 0.10 | 0.00 | ethyl formate |
| OE—CT—CT—HC | 0.00 | 0.00 | 0.34 | 0.00 | as in alcohols |
| Glyoxal analogs | | | | | |
| O═CG—CT/CO—OH | 5.70 | 2.40 | −1.00 | 0.20 | HOP, HMOP, GTHPO |
| CG—CG—CT/CO—OH | 0.00 | 0.00 | 0.00 | 0.00 | HOP, HMOP, GTHPO |
| CG—CT/CO—OH—HO | −0.60 | −0.50 | 0.70 | 0.00 | HOP, HMOP, GTHPO, HMGP |
| O═CG—CT/CO—OS | 0.00 | 0.00 | 0.00 | 0.00 | MOP, HMOP, GTHP, GTHPO |
| CG—CG—CT/CO—OS | −3.50 | 2.00 | 0.40 | 0.30 | MOP, HMOP, GTHP, GTHPO |
| CG—CT/CO—OS—CT | −2.60 | −0.30 | 0.50 | 0.00 | MOP, HMOP, GTHP, GTHPO |
| O═CG—CO—CT | 0.00 | 0.00 | 0.00 | 0.00 | GTHPO |
| CG—CG—CO—CT | −2.00 | 0.75 | 0.40 | 0.00 | GTHPO |

TABLE 6
Properties of bulk liquids. Comparison of OPLS-AA molecular volumes (Å³ per
molecule) and heats of vaporization (kcal/mol) with the experimental data.

| Liquid | T, ° C. | NMOL | R | V_{calc} | Vₑₓₚₜₗ^{a} | ΔH_{vap,calc} | ΔH_{vap,expt}^{b} |
|---|---|---|---|---|---|---|---|
| n-butane | −0.54 | 282 | 14 | 161.4 | 160.6 | 5.58 | 5.36 |
| n-pentane | 25.00 | 267 | 14 | 194.3 | 192.7 | 6.53 | 6.32 |
| isobutane | −12.00 | 332 | 14 | 159.0 | 162.3 | 5.36 | 5.09 |
| isopentane | 25.00 | 267 | 15 | 196.6 | 194.5 | 6.08 | 5.94 |
| neopentane | 9.50 | 267 | 14 | 193.1 | 198.9 | 5.82 | 5.44 |
| cyclopentane | 25.00 | 267 | 15 | 160.8 | 161.7 | 6.85 | 6.83 |
| cyclohexane | 25.00 | 267 | 14 | 182.6 | 180.64 | 8.02 | 7.90 |
| methanol | 25.00 | 539 | 14.5 | 68.4 | 67.66 | 8.92 | 8.94 |
| ethanol | 25.00 | 371 | 15 | 96.5 | 97.43 | 10.15 | 10.13 |
| 1-propanol | 25.00 | 300 | 15 | 124.8 | 124.80 | 11.16 | 11.34 |
| 2-propanol | 25.00 | 333 | 15 | 126.4 | 127.77 | 10.82 | 10.85 |
| sec-butanol | 25.00 | 267 | 15 | 153.8 | 153.05 | 11.78 | 11.91^{c} |
| tert-butanol | 25.00 | 267 | 15 | 153.8 | 157.47 | 11.22 | 11.16 |
| dimethyl ether | −24.80 | 375 | 15 | 106.3 | 104.1 | 5.39 | 5.14 |
| ethyl methyl ether | 7.35 | 293 | 14 | 140.2 | 138.5 | 6.15 | 5.91 |
| isopropyl methyl ether | 25.00 | 267 | 14 | 175.0 | 173.65 | 6.36 | 6.31 |
| methyl tert-butyl ether | 25.00 | 267 | 15 | 201.6 | 199.12 | 7.12 | 7.13 |
| diethyl ether | 25.00 | 267 | 14 | 173.2 | 173.94 | 6.93 | 6.48 |
| oxane | 25.00 | 267 | 15 | 164.5 | 162.74 | 8.56 | 8.35 |
| ^{a}Experimental densities were taken from the following sources: alcohols—reference (Haraschta, P. et al., J Chem Eng Data 1999, 44, 932-935); isobutane—extrapolated from data in (Haynes, W. M. J Chem Eng Data 1983, 28, 367-369); neopentane—extrapolated from data in (McLure, I. A. and Barbarin-Castillo, J. M. Int J Thermophys 1993, 14, 1173-1186), cyclohexane and oxane—reference (Brocos, P. et al., J Chem Eng Data | | | | | | | |
| # 1999, 44, 67-72); aliphatic ethers—reference (Obama, M. et al., J Chem Eng Data 1985, 30, 1-5), except dimethyl ether data which was taken from (Maass, O. and Boomer, E. H. J Am Chem Soc 1922, 44, 1709-1728) and (Kennedy, R. M. et al., J Am Chem Soc 1941, 63, 2267-2272). | | | | | | | |

^{b}Experimental heats of vaporization were obtained from the NIST WebBook (Afeefy, H. Y. et al., Neutral Thermochemical Data; Mallard, W. G. and Linstrom, P. J., Ed.; National Institute of Standards and Technology: Gaithersburg, MD, 2000, (http://webbook.nist.gov))
^{c}Experimental ΔHᵥₐₚ is probably for the racemic mixture while calculations were performed with pure isomer. However, ΔHᵥₐₚ for the pure isomer is expected to be very close to the value of the racemic mixture as the densities and boiling points of the two liquids are nearly identical.

Conformation of Polyketides.

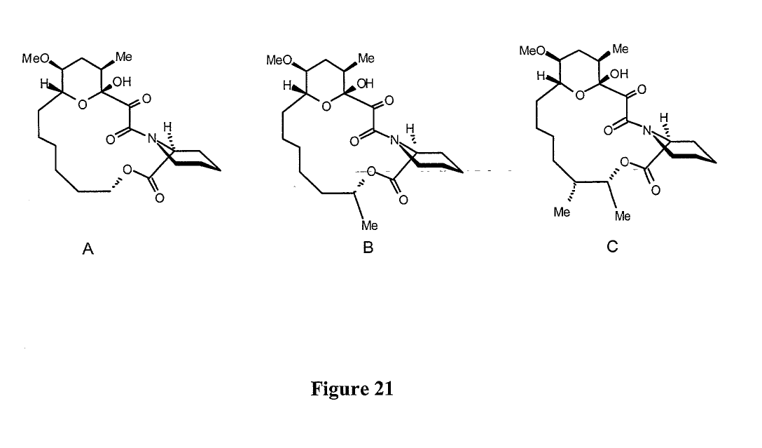

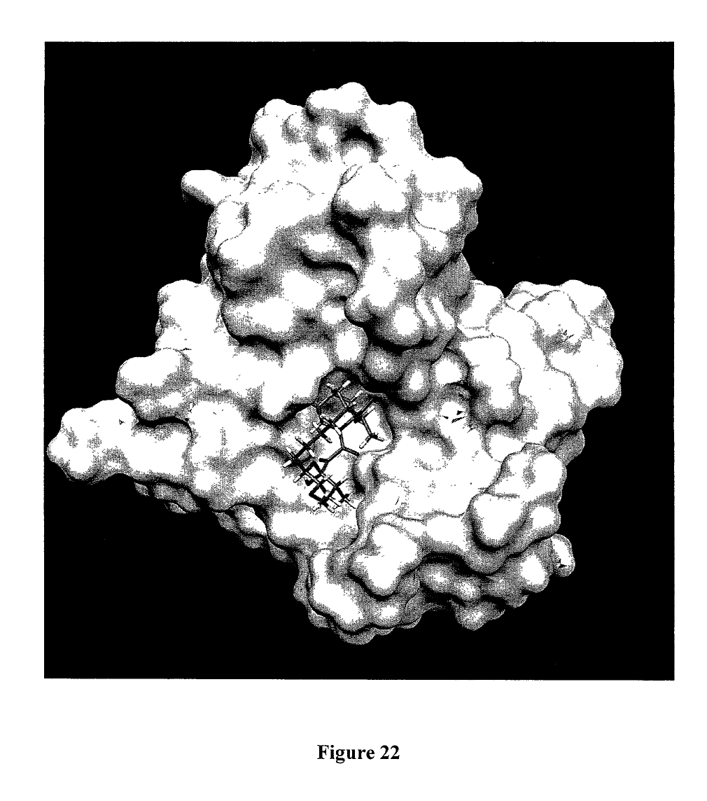

The conformational search was performed for three tetraketides (FIG. 21) as well as for a triketide with unsubstituted linker region. The prefix “tri” and “tetra” refer to the number of ketide units in the linker region. For comparison, the linker in FK506 is built from seven ketide units (Motamedi, H. and Shafiee, A. Eur J Biochem 1998, 256, 528-534). It should be noted that the biosynthesis of tetraketides B and C is in principle possible using acetate as a starter unit. The conformational search of a three tetraketides yielded several hundred conformers for each structure spanning nearly 20 kcal/mol. Despite this structural diversity, the low energy conformers for these molecules were remarkably similar. All of the low energy conformations were characterized by a planar amide group in the trans conformation and by a nearly orthogonal dicarbonyl moiety. The two carbonyl groups were significantly more orthogonal in tetraketides (110° vs. 142°) than in similar acyclic models (Kahn, K. and Bruice, T. C. Bioorg Med Chem 2000, 8:1881-1891), suggesting that a requirement to maintain the covalent ring structure puts a significant torsional stress to the dicarbonyl moiety. The pipecolinyl and pyranosyl rings adopt chair conformations with 11-methyl and 13-methoxy groups in equatorial positions. More importantly, the low energy conformers of these tetraketides were very close to the bound conformation of FK506, suggesting that tetraketides where the linker domain is replaced by a simple aliphatic chain would exhibit significant affinity toward proteins FKBP12 or FKBP52. Docking of the lowest energy conformer of tetraketide C to the binding pocket of the rabbit FKBP52 was achieved using the crystal structure of FKBP12-FK506 as a guide. The tetraketide lies at the bottom of the shallow cavity (FIG. 22) with the pipecolinyl ring deeply buried in the hydrophobic cavity made up of side chains of Val54, Tyr56, Phe76, Trp89, and Tyr112. In contrast, none of the low-energy conformers of the triketide resembled the bound structure of FK506 and could not be docked into the binding pocket without serious steric clashes. These results indicate that the length of the effector domain is more critical than the nature of substituents in the effector domain. If the FKBP52 protein is promiscuous towards the chemical makeup of the effector domain, this site would constitute a suitable region for designing drugs with desired pharmacokinetic properties. For example, substituents that increase the solubility of tetraketide may be introduced to the effector domain without the risk of adversely affecting the binding properties of a drug.

Structural Trends from MP2 Calculations.

The MP2 calculations suggest that the length of the carbon-oxygen single bond in R₁—O—R₂ depends in a systematic way on the nature of substituents R₁ and R₂. For a constant R₂, the length increases in the series methyl<ethyl<isopropyl<tert-butyl. This trend is consistent with the experimental results for alcohols, ethers and esters (Suwa, A. and Ohta, H.; J Mol Struct 1988, 172, 275-290; Egawa, T. et al., J Mol Struct 1993, 298, 37-45; Takeuchi, H. et al.,. J Phys Chem 1993, 97, 7511-7515), and can be rationalized as a sum of steric and inductive effects. For a constant R₁ the bond length increases in the series hemiacetal<ether<alcohol<ester. It was also found that the carbon-carbon bond adjacent to an Sp³ oxygen is considerably shorter than similar bond in hydrocarbons. For example, the C—C distance in ethanol was calculated at 1.504 Å (experimental distance of 1.512 Å has been reported) (Coussan, S. et al., J Phys Chem A 1998, 102, 5789-5793) while this distance in propane was calculated at 1.524 Å (expt. rₛ 1.526 Å) (Lide, D. R. J Chem Phys 1960, 33, 1514-1517). Similar shortening of the carbon-carbon bond was also observed for hemiacetals (r_{C-C} is 1.509 in 1-methoxyethanol), ethers (r_{C-C} is 1.512 Å in ethyl methyl ether) and esters (r_{C-C} is 1.501 Å in ethyl formate). The implication of these results is that force fields which do not allow shrinkage of the C—C bond upon substitution with the electronegative element overestimate this bond length.

Conformational and Structural Data for Individual Molecules.

Alcohols.

The results for methanol and ethanol are in good agreement with previous experimental and high-level computational results (Xu, L. H. et al., J Chem Phys 1999, 110, 3835-3841; Durig, J. R. and Larsen, R. A. J Mol Struct 1989, 238, 195-222; Senent, M. L. et al., J Chem Phys 2000, 112, 5809-5819; Coussan, S. et al., J Phys Chem A 1998, 102, 5789-5793; Iijima, T. J Mol Struct 1989, 212, 137-141; Florián, J.; Leszczynski, J. et al., Mol Physics 1997, 91, 439-447; Adya, A. K. et al., J Chem Phys 2000, 112, 4231-4241). n-Propanol has five distinct conformers which have been studied previously at the MP2/6-31+G(d) and MP4SDQ/TZP levels (Ganguly, B.; Fuchs, B. J Org Chem 1997, 62, 8892-8901; Halgren, T. A.; Nachbar, R. B. J Comput Chem 1996, 17, 587-615). Current MP2/aug-cc-pVTZ optimization predicts, in agreement with previously published results, that the lowest energy conformer is gauche-anti (FIG. 23). However, the four remaining conformers are very close in energy and present results do not allow definite ordering of n-propanol conformers. Since the highest energy conformer lays only 0.23 kcal/mol above the global minimum, it is likely that all conformers are present in significant amounts at room temperature. Isopropanol has two stable conformations, designated here as gauche (a.k.a. synclinal, where HC—OH is ±63.6°) and anti (a.k.a. antiperiplanar, with HC—OH=180°). The gauche conformer is more stable than the anti form but the relative energy of conformers is not precisely known (Schaal, H. et al., J Phys Chem A 2000, 104, 265-274). The MP2/aug-cc-pVTZ optimization predicts that the anti conformer lays 0.27 kcal/mol above the gauche form, in agreement with previously reported theoretical values (Halgren, T. A.; Nachbar, R. B. J Comput Chem 1996, 17, 587-615). 2-Butanol (FIG. 24) exists in nine conformers, which have been recently studied at the B3LYP/6-31 G(d) level (Xu, S. et al., J Phys Chem A 2000, 104, 8671-8676; Wang, F.; Polavarapu, P. L. J Phys Chem A 2000, 104, 10683-10687). Our results conform that all nine conformers are present in significant amounts at room temperature, but the identity of the global minimum remains elusive. The MP2/TZ//DZ calculations predict that the aa conformer is the most stable, followed closely by the ag⁻ conformer, while DFT calculations predict that the ag⁺ form is the global minimum. This molecule is an important model for the C—C—C—O moiety in C-glycosides and further studies of its conformational properties may be necessary.

Ethers.

Ethyl methyl ether has two stable conformers and experimental estimates for the anti-gauche energy difference range from 1.11 to 1.5 kcal/mol (Tsuzuki, S. et al., J Mol Struct (THEOCHEM) 1996, 366, 89-96; Durig, J. R. et al., J Chem Phys 1978, 69, 4713-4719). This molecule has been well studied by ab initio calculations (Tsuzuki, S. et al., J Mol Struct (THEOCHEM) 1996, 366, 89-96; Halgren, T. A.; Nachbar, R. B. J Comput Chem 1996, 17, 587-615) and our MP2/TZ//DZ value, 1.37 kcal/mol, is very close to the best available estimate, 1.36 kcal/mol at the CCSDT/6-311G(d) level (Tsuzuki, S. et al.; J Mol Struct (THEOCHEM) 1996, 366, 89-96). The calculated barrier for the C—CH₃ rotation, 3.08 kcal/mol, also is in a good agreement with experimental estimates of 3.08 and 3.14 kcal/mol (Durig, J. R. et al., J Mol Struct 1970, 6, 457-470). However, there is a notable disagreement between the calculated and experimental values for the CC—OC dihedral angle in the gauche conformer. The electron diffraction data (Oyanagi, K.; Kuchitsu, K. Bull Chem Soc Jpn 1978, 51, 2237-2242) suggest that this torsional angle is 84±6° while the MP2/aug-cc-pVTZ optimization yields a value of 70.5°. It is likely that the electron diffraction value is in error, especially in the light of infrared spectral measurements, which yield a potential energy curve with the minimum at 64° (Durig, J. R.; Compton, D. A. C. J Chem Phys 1978, 69, 4713-4719). Methyl propyl ether (FIG. 25) has four conformers, ga, aa, gg, and ag, which have been studied previously at the MP2/6-31G(d)//MP2/3-21G(d) level (Gil, F. P. S. C.; Teixeira-Dias, J. J. C. J Mol Struct (THEOCHEM) 1996, 363, 311-317). The MP2/TZ//DZ method predicts, in agreement with previous theoretical results, that the ga conformer (CC—CO 60°, CC—OC −178°) is the global minimum followed by the aa conformer. It should be noted that the experimental structure determination of methyl propyl ether rests on the assumption that the aa conformer is the dominant species (Kato, H. et al., J Mol Spectrosc 1980, 80, 272-278). If the ga conformer is indeed the global energy minimum, reanalysis of structural data for this compound may be needed. The barrier to the internal rotation in secondary aliphatic ethers is significantly smaller than in primary ethers. The calculated barrier in isopropyl methyl ether, 1.80 kcal/mol, is in good agreement with the experimental value of 1.73 kcal/mol (Nakagawa, J. et al., J Mol Struct 1984, 112, 201-206).

Hemiacetals.

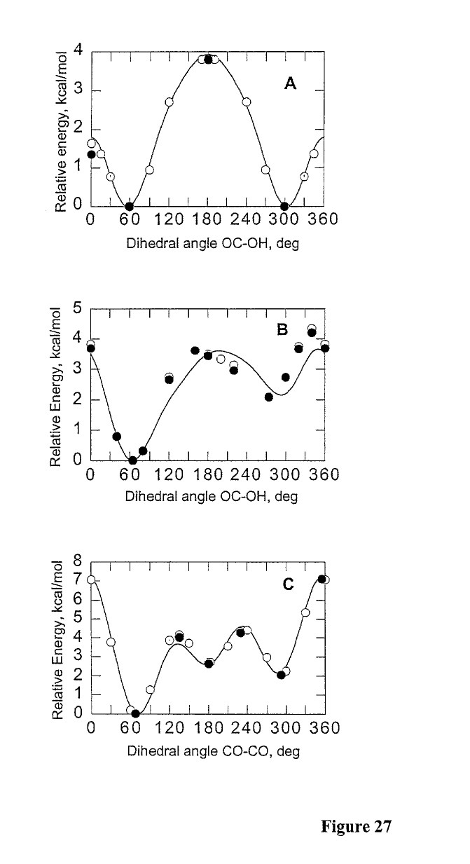

Methoxymethanol is the simplest hemiacetal and several previous studies have looked into the conformational properties of this molecule (Ganguly, B.; Fuchs, B. J Org Chem 1997, 62, 8892-8901; Halgren, T. A.; Nachbar, R. B. J Comput Chem 1996, 17, 587-615). It is an important model for the anomeric effect (the O—C—O unit prefers a gauche orientation) and methoxymethanol has been used to parameterize force fields for carbohydrates. The g⁺g⁺ conformer is the global minimum for methoxymethanol (FIG. 26). The two other minima at the MP2/TZ//DZ potential energy surface are g⁻g⁺ (2.05 kcal/mol) and ag⁺ (2.64 kcal/mol). Interestingly, the aa form (6.43 kcal/mol) was found to be a transition state at this level of theory instead of a high energy minimum as was previously thought (Ganguly, B.; Fuchs, B. J Org Chem 1997, 62, 8892-8901). The ga structure was not a local minimum at the MP2/aug-cc-pVDZ level (FIG. 27), in accord with previous MP2/6-31+G(d) results (Ganguly, B.; Fuchs, B. J Org Chem 1997, 62, 8892-8901). FIG. 27 displays energy profiles for the rotation around the HO—CO and CO—CO bonds in methoxymethanol. It was also found that the rotation of the methyl group around the O—CH₃ bond in hemiacetals faces significantly lower energy barrier than a similar rotation in ethers (Table 3). Six stable conformers were located for methoxyethanol (FIG. 28) and five energy minima were found on the energy surface of tetrahydropyran-2-ol (FIG. 29). The anti orientation of the hydroxyl group in equatorial tetrahydropyran-2-ol was a transition state at the MP2/aug-cc-pVDZ level. Interestingly, the structure with the H—O—C—O dihedral at 171° and an axial hydroxyl group was a local minimum.

Esters.

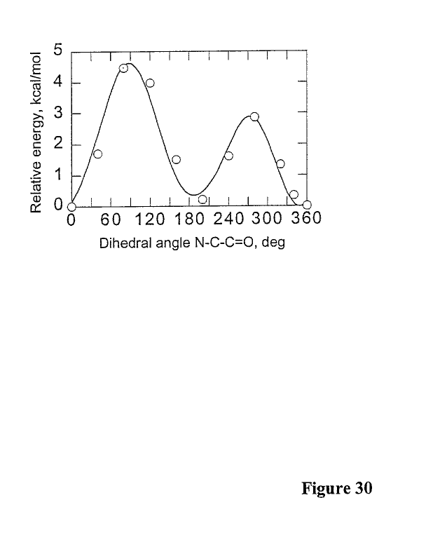

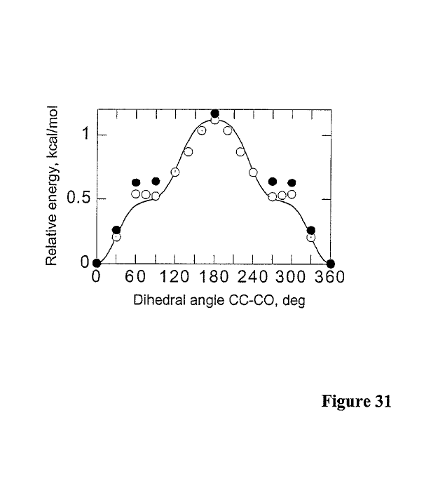

The study of the ester moiety was inspired by the need to know the torsional barrier in pipecolinyl esters, which is a part of the polyketide structure. This molecule presents a unique N(amide)-CT—C═O dihedral where the fragment N(amide)-CT is part of the pipecolinyl ring. The torsional profile for this molecule is shown in FIG. 30. In order to assign force field parameters for this dihedral, torsional profiles for the CT—CT—C═O and HC—CT—C═O dihedrals were also independently determined for methyl propionate and methyl acetate. Methyl propionate has been studied with the MP2 and DFT methods employing the 6-31G(d) basis set (Blomqvist, J. et al., J Mol Struct (THEOCHEM) 1999, 488, 247-262); we find that MP2/TZ/DZ yields a slightly lower torsional barrier (FIG. 31) than the previously reported one. We have performed a limited number of calculations on other esters in order to verify the suitability of the MP2/TZ/DZ method. More extensive study of rotational profiles in esters has been published very recently using HF/6-31G(d) method (Price, M. L. et al., J Comput Chem 2001, 22, 1340-1352). In accord with the literature data, the ester moiety can adopt two orientations around the C(sp²)—O(sp³) bond with the Z (cis) form being more stable. Barriers for the rotation around this bond are known to be ca. 10-15 kcal/mol from the Z conformer (Blom, C. E. et al., Chem Phys Lett 1981, 84, 267-271; Blomqvist, J. et al., J Mol Struct (THEOCHEM) 1999, 488, 247-262). The MP2 energy difference between Z and E conformers of methyl formate (5.45 kcal/mol) is larger than the experimental value 4.75±0.19 but the energy difference for methyl acetate (7.47 kcal/mol) is in the lower end of the experimental value 8.5±1.0 (Blom, C. E.; Günthard, H. H. Chem Phys Lett 1981, 84, 267-271). The structure of methyl acetate at the MP2/aug-cc-pVTZ level is very similar to the experimental geometry from joint electron diffraction-microwave-infrared analysis (Pyckhout, W. et al., J Mol Struct 1986, 144, 265-279). The barrier for the C—CH₃ rotation in ethyl acetate was found to be 0.19 kcal/mol, this can be compared to the experimental value of 0.29 kcal/mol (Sheridan, J. et al., J Mol Spectrosc 1980, 80, 1-11). The barrier for the rotation around the O—CH₃ bond depends on the conformation around the C(sp²)—O(sp³) bond (Wiberg, K. B. et al., J Mol Struct 1999, 485-486, 239-248). This barrier is about 1.2 kcal/mol in Z forms of methyl esters while negligible barrier is present in E-conformers. The MP2 calculations reproduce these features very well and it appears that the MP2/TZ//DZ method is reliable in calculating the conformational energies of esters.

Glyoxal analogs.

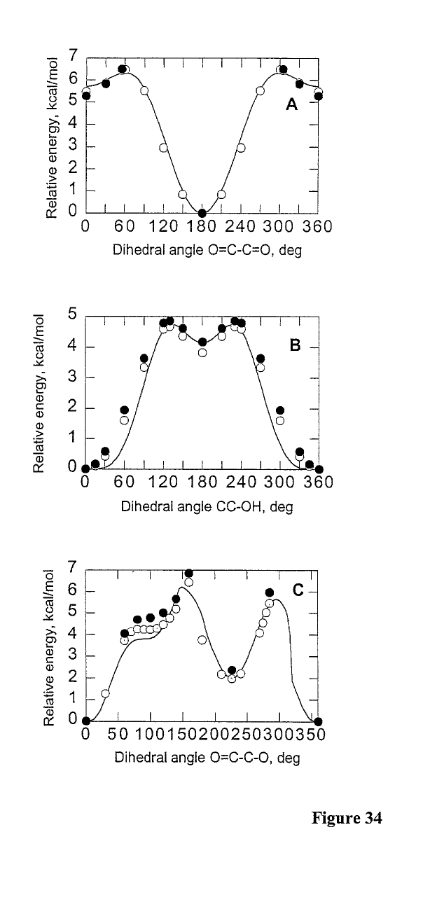

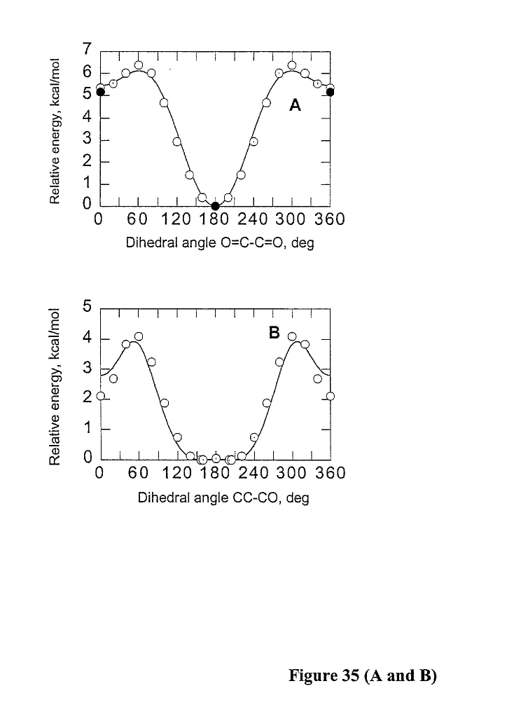

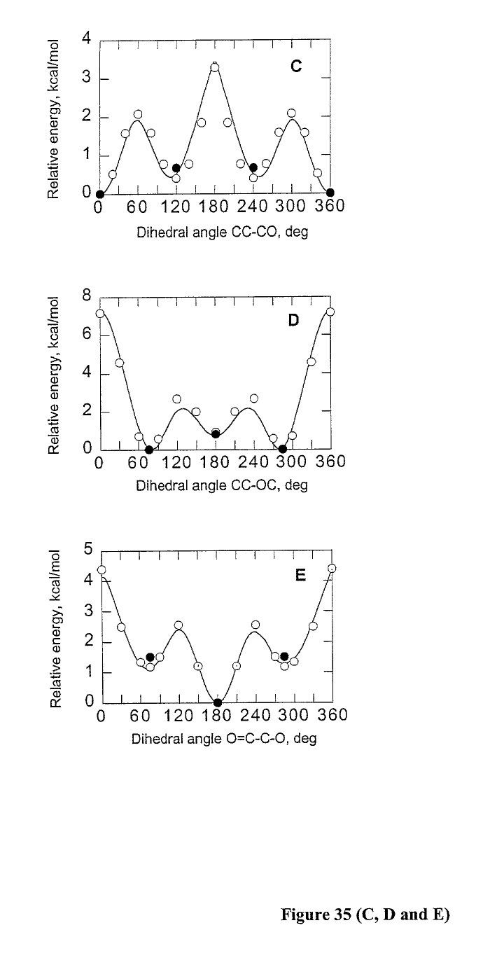

Ab initio studies on glyoxal analogs have been published recently (Kahn, K. and Bruice, T. C. Bioorg Med Chem 2000, 8, 1881-1891). This application describes five additional analogs, which, along with the data on alcohols, hemiacetals, and ethers, allowed derivation of torsional parameters for the pyranosyl moiety of polyketides. Seven conformations were found for 3-hydroxy-2-oxopropanal (pyruvaldehyde) at the MP2/aug-cc-pVDZ level. The lowest energy conformer (FIG. 32) showed CS symmetry and an intramolecular hydrogen bond between the hydroxyl hydrogen and an adjacent carbonyl group. In the second conformer, the intramolecular hydrogen bond was to a distal carbonyl group. The analogous molecule, 3-methoxy-2-oxopropanal, had eight stable conformers (FIG. 33) but because of the lack of intramolecular hydrogen bonding, the relative energy ordering is quite different than in the hydroxyl analog. The torsional profiles for 3-hydroxy-2-oxopropanal and 3-methoxy-2-oxopropanal are shown in FIG. 34 and FIG. 35, respectively. Four low-energy conformers were found for 3-hydroxy-3-methoxy-2-oxopropanal (FIG. 36) and for 2-glyoxoyl-tetrahydropyran (FIG. 37), and six minima were located for 2-glyoxoyl-tetrahydropyran-2-ol (FIG. 38). It is likely that additional high-energy minima for these molecules exist. It is noteworthy that in 3-hydroxy-3-methoxy-2-oxopropanal and 2-glyoxoyl-tetrahydropyran-2-ol the structures with a hydrogen bond between the hydroxyl group and distal carbonyl is a global minimum, contrary to the order of stabilities in 3-hydroxy-2-oxopropanal. This effect cannot be reproduced accurately with the original nor present OPLS-AA parameterization and appears to be a deficiency in treatment of nonbonded interactions.

Accuracy of the Force Field.

Recent comparison of the several common force fields has shown that conformational energies are often predicted with RMS deviations in excess of 1 kcal/mol from experimental or accurate ab initio data (Halgren, T. A. J Comput Chem 1999, 20, 730-748). It has also been demonstrated that accuracy of predictions can be increased significantly by careful parameterization of force constants based on ab initio data and RESP charges (Wang, J. et al., J Comp Chem 2000, 21, 1049-1074). Comparison of present OPLS-AA and MP2 conformational energies from Tables 3 and 7 shows that, when supplied with proper torsional parameters, the OPLS-AA force field can accurately reproduce conformational energies of organic molecules containing sp³ carbons and oxygens in various molecular environments. Significant improvement over the standard OPLS-AA parameters is seen with alcohols, ethers, and hemiacetals. For alcohols (see Supplementary Material) the maximum error is reduced from 2.03 kcal/mol to 0.93 kcal/mol and RMS deviation has decreased from 0.56 kcal/mol to 0.34 kcal/mol. The maximum deviation among all molecules, 1.42 kcal/mol, is found for the energy difference between the twist-boat and chair conformers of oxane. Overall RMS deviation of OPLS-AA data from the reference (either MP2/TZ//DZ or previously published data) conformational energies and torsional barrier heights is 0.39 kcal/mol for alkanes, alcohols, ethers, and hemiacetals (Table 7). Cartesian coordinates for all stable conformers for molecules discussed herein are provided in Table 8. Additionally, comparison between the current and standard OPLS-AA parameters in predicting relative conformational energies of alcohols is provided in Table 9. Thus, current parameters give conformational energies with quality similar to the MMFF (Halgren, T. A. J Comput Chem 1999, 20, 730-748) and recently reparameterized RESP-based AMBER (Wang, J. et al., J Comp Chem 2000, 21, 1049-1074). It should be pointed out that Jorgensen has very recently proposed a new set of alkane torsional parameters which appears of comparable quality to parameters reported here (Price, M. L. et al., J Comput Chem 2001, 22, 1340-1352).

TABLE 7
Summary comparison of ab initio and OPLS-AA conformational energies.

| | MP2/DZ vs. MP2/TZ//DZ | | | OPLS-AA vs. TARGET^{a} | | |
|---|---|---|---|---|---|---|
| Class | Max^{b} | RMSD | No | Max | RMSD | No |
| Alkanes | 0.22 | 0.10 | 11 | 0.93 | 0.42 | 19 |
| Alcohols | 0.29 | 0.16 | 36 | 0.99 | 0.34 | 36 |
| Ethers | 0.29 | 0.11 | 15 | 1.42 | 0.42 | 19 |
| Hemiacetals | 0.18 | 0.09 | 12 | 1.39 | 0.48 | 17 |
| Esters | 0.20 | 0.11 | 14 | 1.17 | 0.56 | 17 |
| Glyoxals | 0.55 | 0.27 | 17 | 1.87 | 0.75 | 17 |
| Overall | 0.55 | 0.16 | 106 | 1.87 | 0.48 | 126 |

^{a}The MP2/TZ//DZ data, when available, were used for target values. When the MP2/TZ//DZ data was not available, previously published reliable experimental or ab initio data from Table 3 were used. For tetrahydropyran-2-ol, MP2/aug-cc-pVDZ relative energies were used.
^{b}Abbreviations used: Max—maximum absolute difference of conformational energies (kcal/mol), RMSD—root mean square deviation (kcal/mol), No—number of relative energies values that were included in the comparison.

Statistical analysis of bulk properties of alkanes, alcohols, and ethers with current parameters shows that molar volumes of liquids from simulations are on average within 1.1% of the corresponding experimental values. The largest error, 2.9%, occurs with neopentane. We note that the experimental molar volume of neopentane at 9.5° C. has not been directly measured but was obtained by extrapolating the experimental density data. The mean error for heats of vaporization of nineteen compounds from Table 6 is 2.4%. Notable deviations from experimental data occur with neopentane and diethyl ether. Overall, current parameters perform slightly better than the original OPLS-AA parameterization at the expense of additional nonbonded parameters for secondary and tertiary carbons. This allows, among other things, a correct prediction of the relative densities and heats of vaporization of 1-propanol and 2-propanol.

Tetrakedides as Potent Neuroregenerative Agents.

The main finding pertaining to the design of neuroregenerative drugs is that tetraketides with minimally modified linker region are predicted to adopt low-energy conformations which are similar to the drug FK506 bound to protein FKBP12. Thus, they are likely to bind tightly to protein FKBP52 as well, and may possess neuroregenerative activity. By comparing the structures obtained with this method to current conformers, we note that the torsion around the N—C—C═O bond in pipecolinyl esters is described very differently by the two force fields. Based on ab initio calculations, OPLS-AA predicts potential minima near zero and 180 degrees while MM3 yields energy minimum at around 90 degrees. This behavior was also found in the pipecolinyl ester model MPIP for which MM3 predicted a minimum with N—C—C═O dihedral at −73°. This discrepancy arises most likely from the fact that the N—C—C═O dihedral in the MM3 is parameterized based on peptides. This torsional parameter is apparently not transferable from peptides to pipecolinyl esters and thus MM3 force field does not describe this portion of polyketides accurately. Conversely, the current polyketide N—C—C═O torsional parameter should not be used for peptides.

In summary, this example utilized high-level ab initio structures, conformational energies, and torsional energy profiles, along with Monte Carlo liquid simulations, to derive new parameters for the OPLS-AA force field. Conformational analysis of polyketides using this force field suggested that tetraketides containing simple aliphatic effector domain would bind the target protein FKBP with an affinity similar to FK506 while not interacting with calcineurin. These properties, along with the possibility of synthesis of tetraketides via engineered biosynthesis, make tetraketides attractive neuroregenerative agents that lack the immunosuppressive side effect.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

TABLE 9
Comparison between the current and the standard OPLS-AA parameters in
predicting relative conformational energies of alcohols.

| Compound | MP2/TZ//DZ | Other | Current | Standard¹ |
|---|---|---|---|---|
| Methanol (s_{TS}-a) | 1.00 | 1.065^{2,3} | 1.03 | 1.36 |
| Ethanol (g-a) | 0.22 | 0.13⁴; 0.05⁵ | 0.29 | 0.09 |
| Ethanol (s_{TS}-a) | 1.32 | 1.27⁴; 1.26⁵ | 1.48 | 1.76 |
| Ethanol (e_{TS}-a) | 1.15 | 1.15⁴; 1.15⁵ | 1.07 | 1.32 |
| Ethanol (CH₃:a) | 3.30 | 3.39⁴; 3.31⁵ | 3.20 | 3.59 |
| 1-PrOH (aa-g⁻a) | 0.17 | | −0.34 | 0.06 |
| 1-PrOH (g⁻g⁻-g⁻a) | 0.20 | | −0.30 | −0.25 |
| 1-PrOH (ag-g⁻a) | 0.22 | | −0.13 | −0.04 |
| 1-PrOH (g⁻g⁺-g⁻a) | 0.23 | | 0.03 | −0.71 |
| 1-PrOH (sa_{TS}-g⁻a) | 5.11 | | 4.85 | 5.37 |
| 1-PrOH (ea_{TS}-g⁻a) | 3.35 | | 3.40 | 3.76 |
| 1-PrOH (g⁻e⁺_{TS}-g⁻a) | 0.82 | | 0.86 | 1.06 |
| 1-PrOH (g⁻e⁻_{TS}-g⁻a) | 0.96 | | 1.05 | 1.31 |
| 1-PrOH (sg⁻_{TS}-g⁻g⁻) | 4.98 | | 4.45 | 5.30 |
| 1-PrOH (e⁺g⁻_{TS}-g⁻g⁻) | 3.72 | | 3.33 | 4.14 |
| 1-PrOH (e⁻g⁻_{TS}-g⁻g⁻) | 3.52 | | 3.60 | 4.48 |
| 1-PrOH (as_{TS}-aa) | 1.08 | | 1.40 | 1.67 |
| 1-PrOH (ae_{TS}-aa) | 0.95 | | 1.03 | 1.30 |
| 1-PrOH (CH₃:aa) | 2.64 | 2.73 ± 0.06⁶ | 2.45 | 2.44 |
| 2-PrOH (a-g) | 0.35 | 0.28⁷; 0.45 ± 0.22⁸ | 0.28 | 0.07 |
| 2-PrOH (s_{TS}-g) | 1.33 | | 0.80 | 1.17 |
| 2-PrOH (e_{TS}-g) | 1.18 | | 1.21 | 1.62 |
| 2-PrOH (CH₃:g) | 3.30 | | 3.44 | 3.78 |
| tert-Butanol (s_{TS}-a) | 1.21 | 1.27⁹ | 0.96 | 1.48 |
| tert-Butanol (CH₃:a) | 3.46 | | 3.77 | 4.08 |
| 2-ButOH (ag⁻-aa) | 0.06 | −0.14^{10,11} | −0.60 | −0.82 |
| 2-ButOH (ag⁺-aa) | 0.28 | −0.18^{10,11} | 0.03 | −0.28 |
| 2-ButOH (g⁻g⁻-aa) | 0.32 | 0.65^{10,11} | 0.28 | 0.74 |
| 2-ButOH (g⁻a-aa) | 0.44 | 0.73 ^{10,11} | 0.36 | 0.86 |
| 2-ButOH (g⁺g⁻-aa) | 0.60 | 0.97^{10,11} | 0.52 | 0.58 |
| 2-ButOH (g⁻g⁺-aa) | 0.64 | 0.61^{10,11} | 0.55 | 0.81 |
| 2-ButOH (g⁺a-aa) | 0.70 | 1.13^{10,11} | 0.80 | 0.96 |
| 2-ButOH (g⁺g⁺-aa) | 1.05 | 1.24^{10,11} | 0.48 | 0.16 |
| cyclohexanol (eq/CS-eq/C1) | 0.26 | 0.18⁷ | 0.14 | −0.12 |
| cyclohexanol (ax/C1-eq/C1) | 0.45 | 0.33⁷ 10.58⁷ | 1.07 | 0.64 |
| cyclohexanol (ax/CS-eq/C1) | 1.20 | 1.14⁷ | 0.21 | −0.83 |

### REFERENCES FOR TABLE 9

1) Jorgensen, W. L.; Maxwell, D. S.; Tirado-Rives, J. J Am Chem Soc 1996, 118, 11225-11236.

2) De Lucia, F. C.; Herbst, E.; Anderson, T.; Helminger, P. J Mol Spectrosc 1989, 134, 395-411.

3) Xu, L. H.; Lees, R. M.; Hougen, J. T. J Chem Phys 1999, 110, 3835-3841.

4) Durig, J. R.; Larsen, R. A. J Mol Struct 1989, 238, 195-222.

5) Senent, M. L.; Smeyers, Y. G.; Dominguez-Gómez, R.; Villa, M. J Chem Phys 2000, 112, 5809-5819.

6) Dreizler, H.; Scappini, F. Z. Naturforsch 1981, 36A, 1187-1191.

7) Halgren, T. A. J Comput Chem 1999, 20, 730-748.

8) Hirota, E. J. Phys Chem 1979, 83, 1457-1465.

9) Suwa, A.; Ohta, H.; J Mol Struct 1988, 172, 275-290.

10)Xu, S.; Liu, Y.; Sha, G.; Zhang, C.; Xie, J. J Phys Chem A 2000, 104, 8671-8676.

11) Wang, F.; Polavarapu, P. L. J Phys Chem A 2000, 104, 10683-10687.

Various publications are cited herein that are hereby incorporated by reference in their entirety.

As will be apparent to those skilled in the art in which the invention is addressed, the present invention may be embodied in forms other than those specifically disclosed without departing from the spirit or potential characteristics of the invention. Particular embodiments of the present invention described above are therefore to be considered in all respects as illustrative and not restrictive. The scope of the invention is as set forth in the appended claims and equivalents thereof, rather than being limited to the examples contained in the foregoing description.

What we claim is:

1. A method for obtaining novel polyketide compounds having a selected bioactivity, comprising:
a) generating a virtual library of polyketide compounds having a binding domain and an effector domain, said compounds containing modifications of the effector domain of a native polyketide compound, said modifications comprising introducing deletions or replacements of at least one ketide unit in the effector domain of the native polyketide compound, wherein the effector domain of the polyketide compound comprises a linker of four ketide units;
b) screening the virtual library for compounds having the desired bioactivity; and
c) synthesizing compounds obtained from the screening of step (b).

2. The method of claim 1, wherein said deletions or replacements are from one to three ketide units in the effector domain of the native polyketide compound.

3. The method of claim 1, wherein the step of screening comprises selecting for compounds that bind a first compound but not a second compound.

4. The method of claim 3, wherein the first compound is FKBP12 or FKBP52, and the second compound is FRAP or Calcineurin.

5. The method of claim 1, wherein the native polyketide compound is Rapamycin or FK506.

6. The method of claim 1, wherein the desired bioactivity is neuroregenerative activity.

7. The method of claim 6, wherein the polyketide compound lacks immunosuppressive activity.

8. Polyketide compounds obtained by the method of claim 1.

9. A polyketide compound having a selected bioactivity, wherein the compound comprises a binding domain and an effector domain having deletions or replacements of at least one ketide unit in the effector domain, wherein the effector domain comprises a linker of four ketide units.

10. The compound of claim 9, wherein the compound has a structure as shown in FIGS. 21A, 21B, or 21C.

11. The compound of claim 9 having neuroregenerative activity but lacking immunosuppressive activity.

12. A method of regenerating neurons, comprising contacting neurons with the polyketide compound of claim 11.
